SEQUENCE LISTING (40 sequences)

SEQ ID NO: 1 - protein, 17 aa, Artificial Sequence
  An exemplary sequence of a linker
SSGGGGSGGGGSGGGGS

SEQ ID NO: 2 - protein, 242 aa, Artificial Sequence
  An exemplary sequence of a scFV that binds CD19
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 3 - protein, 247 aa, Artificial Sequence
  An exemplary sequence of a scFV that binds CD20
GDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 4 - protein, 22 aa, Artificial Sequence
  An exemplary sequence of a signal peptide
MDFQVQIFSFLLISASVIMSRG

SEQ ID NO: 5 - protein, 22 aa, Artificial Sequence
  An exemplary sequence of a signal peptide
LLVTSLLLCELPHPAFLLIPDT

SEQ ID NO: 6 - protein, 236 aa, Artificial Sequence
  An exemplary sequence of a spacer domain
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 7 - protein, 45 aa, Artificial Sequence
  An exemplary sequence of a CD8 hinge domain
TTTPAPRPPTPAPTIASQPLSLRPEACRPAAGGAVHTRGLDFACD

SEQ ID NO: 8 - protein, 24 aa, Artificial Sequence
  An exemplary sequence of CD8 transmembrane domain
IYIWAPLAGTCGVLLLSLVITLYC

SEQ ID NO: 9 - protein, 27 aa, Artificial Sequence
  An exemplary sequence of a CD28 transmembrane domain
FWVLVVVGGVLACYSLLVTVAFIIFWV

SEQ ID NO: 10 - protein, 78 aa, Artificial Sequence
  Amino acid 224-301 of human TIM-3
FKWYSHSKEKIQNLSLISLANLPPSGLANAVAEGIRSEENIYTIEENVYEVEEPNEYYCYVSSRQQPSQPLGCRFAMP

SEQ ID NO: 11 - protein, 54 aa, Artificial Sequence
  Human TIM-3 cytoplasmic domain lacking amino acid residues 278-301
FKWYSHSKEKIQNLSLISLANLPPSGLANAVAEGIRSEENIYTIEENVYEVEEP

SEQ ID NO: 12 - protein, 40 aa, Artificial Sequence
  Human TIM-3 cytoplasmic domain lacking amino acid residues 264-301
FKWYSHSKEKIQNLSLISLANLPPSGLANAVAEGIRSEEN

SEQ ID NO: 13 - protein, 78 aa, Artificial Sequence
  A variant human TIM-3 cytoplasmic domain
  MISC_FEATURE (42)..(42): Xaa = tyrosine or alanine
  MISC_FEATURE (49)..(49): Xaa = tyrosine or alanine
  MISC_FEATURE (57)..(58): Xaa = tyrosine or alanine
  MISC_FEATURE (60)..(60): Xaa = tyrosine or alanine
FKWYSHSKEKIQNLSLISLANLPPSGLANAVAEGIRSEENIXTIEENVXEVEEPNEXXCXVSSRQQPSQPLGCRFAMP

SEQ ID NO: 14 - protein, 112 aa, Artificial Sequence
  An exemplary sequence of a CD3 zeta signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 84 aa, Artificial Sequence
  An exemplary sequence of a CD8 signaling domain
FVPVFLPAKPTTTPAPRPPTPAPTIASQPLSLRPEACRPAAGGAVHTRGLDFACDIYIWAPLAGTCGVLLLSLVITLYCNHRNR

SEQ ID NO: 16 - protein, 40 aa, Artificial Sequence
  An exemplary sequence of a CD28 signaling domain
SKRSRLLHSDYMNMTPRRPGPTRKHYQPYAPPRDFAAYRS

SEQ ID NO: 17 - protein, 42 aa, Artificial Sequence
  An exemplary sequence of a CD137 signaling domain
KRGRKKLLYIFKQPFMRPVQTTQEEDGCSCRFPEEEEGGCEL

SEQ ID NO: 18 - protein, 47 aa, Artificial Sequence
  An exemplary sequence of a CD137 signaling domain
RFSVVKRGRKKLLYIFKQPFMRPVQTTQEEDGCSCRFPEEEEGGCEL

SEQ ID NO: 19 - DNA, 2355 bp, Artificial Sequence
  An exemplary sequence of the coding region of pHR-EF1-aCD20-TIM-3z-T2A-TagBFP
atggagacagacacactcctgctatgggtgctgctgctctgggttccaggttccacaggtggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcaaccactactccggcaccgcgccccccaactcctgcaccgacgatagcttcacaaccgctttcattgcggcccgaagcatgtcggccagccgccggaggcgctgtgcatacaagagggctggattttgcatgtgatatatatatttgggcgccccttgctggcacttgcggcgttcttcttcttagcctcgttattacgctctactgtggcttcaagtggtattctcactccaaggaaaagattcaaaacctctcattgattagccttgccaaccttccccctagcggcctcgccaacgcggtggccgaggggatcagaagtgaagaaaacatctacaccatcgaagaaaatgtttacgaagttgaagaaccaaacgaatactattgttacgttagctcccgacaacagccttcacagccacttggctgtcgcttcgccatgccgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctcgcatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgaaacatacgtcgagcagcacgaggtggcagtggccagatactgcgacctccctagcaaactggggcacaagcttaattaa

SEQ ID NO: 20 - DNA, 2340 bp, Artificial Sequence
  An exemplary sequence of the coding region of pHR-EF1-aCD19-TIM-3z-T2A-TagBFP
atggagacagacacactcctgctatgggtgctgctgctctgggttccaggttccacaggtgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccactactccggcaccgcgccccccaactcctgcaccgacgatagcttcacaaccgctttcattgcggcccgaagcatgtcggccagccgccggaggcgctgtgcatacaagagggctggattttgcatgtgatatatatatttgggcgccccttgctggcacttgcggcgttcttcttcttagcctcgttattacgctctactgtggcttcaagtggtattctcactccaaggaaaagattcaaaacctctcattgattagccttgccaaccttccccctagcggcctcgccaacgcggtggccgaggggatcagaagtgaagaaaacatctacaccatcgaagaaaatgtttacgaagttgaagaaccaaacgaatactattgttacgttagctcccgacaacagccttcacagccacttggctgtcgcttcgccatgccgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctcgcatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgaaacatacgtcgagcagcacgaggtggcagtggccagatactgcgacctccctagcaaactggggcacaagcttaattaa

SEQ ID NO: 21 - protein, 784 aa, Artificial Sequence
  Sequence encoded by pHR-EF1-aCD20-TIM-3z-T2A-TagBFP
METDTLLLWVLLLWVPGSTGGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSTTTPAPRPPTPAPTIASQPLSLRPEACRPAAGGAVHTRGLDFACDIYIWAPLAGTCGVLLLSLVITLYCGFKWYSHSKEKIQNLSLISLANLPPSGLANAVAEGIRSEENIYTIEENVYEVEEPNEYYCYVSSRQQPSQPLGCRFAMPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 22 - protein, 779 aa, Artificial Sequence
  Sequence encoded by pHR-EF1-aCD19-TIM-3z-T2A-TagBFP
METDTLLLWVLLLWVPGSTGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCGFKWYSHSKEKIQNLSLISLANLPPSGLANAVAEGIRSEENIYTIEENVYEVEEPNEYYCYVSSRQQPSQPLGCRFAMPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 23 - protein, 8 aa, Artificial Sequence
  Sequence of a CDR of a scFV that binds CD19
GVSLPDYG

SEQ ID NO: 24 - protein, 7 aa, Artificial Sequence
  Sequence of a CDR of a scFV that binds CD19
IWGSETT

SEQ ID NO: 25 - protein, 14 aa, Artificial Sequence
  Sequence of a CDR of a scFV that binds CD19
AKHYYYGGSYAMDY

SEQ ID NO: 26 - protein, 6 aa, Artificial Sequence
  Sequence of a CDR of a scFV that binds CD19
QDISKY

SEQ ID NO: 27 - protein, 9 aa, Artificial Sequence
  Sequence of a CDR of a scFV that binds CD19
QQGNTLPYT

SEQ ID NO: 28 - protein, 8 aa, Artificial Sequence
  Sequence of a CDR of a scFV that binds CD20
GYTFTSYN

SEQ ID NO: 29 - protein, 8 aa, Artificial Sequence
  Sequence of a CDR of a scFV that binds CD20
IYPGNGDT

SEQ ID NO: 30 - protein, 15 aa, Artificial Sequence
  Sequence of a CDR of a scFV that binds CD20
ARSNYYGSSYWFFDV

SEQ ID NO: 31 - protein, 5 aa, Artificial Sequence
  Sequence of a CDR of a scFV that binds CD20
SSVNY

SEQ ID NO: 32 - protein, 9 aa, Artificial Sequence
  Sequence of a CDR of a scFV that binds CD20
QQWSFNPPT

SEQ ID NO: 33 - protein, 27 aa, Artificial Sequence
  Sequence of a 4-1BB transmembrane domain
IISFFLALTSTALLFLLFFLTLRFSVV

SEQ ID NO: 34 - protein, 68 aa, Artificial Sequence
  Sequence of a TIM-3 cytoplasmic domain
FKWYSHSKEKIQNLSLISLANLPPSGLANAVAEGIRSEENIYTIEENVYEVEEPNEYYCYVSSRQQPS

SEQ ID NO: 35 - protein, 41 aa, Artificial Sequence
  Sequence of a TIM-3 cytoplasmic domain
FKWYSHSKEKIQNLSLISLANLPPSGLANAVAEGIRSEENI

SEQ ID NO: 36 - protein, 78 aa, Artificial Sequence; identical to SEQ ID NO: 13
  Sequence of a TIM-3 cytoplasmic domain
  MISC_FEATURE (42)..(42): Xaa = tyrosine, alanine, phenylalanine, aspartic acid, or glutamic acid
  MISC_FEATURE (49)..(49): Xaa = tyrosine, alanine, phenylalanine, aspartic acid, or glutamic acid
  MISC_FEATURE (57)..(58): Xaa = tyrosine, alanine, phenylalanine, aspartic acid, or glutamic acid
  MISC_FEATURE (60)..(60): Xaa = tyrosine, alanine, phenylalanine, aspartic acid, or glutamic acid

SEQ ID NO: 37 - protein, 68 aa, Artificial Sequence
  Sequence of a TIM-3 cytoplasmic domain
  MISC_FEATURE (42)..(42): Xaa = tyrosine, alanine, phenylalanine, aspartic acid, or glutamic acid.
  MISC_FEATURE (49)..(49): Xaa = tyrosine, alanine, phenylalanine, aspartic acid, or glutamic acid.
  MISC_FEATURE (57)..(58): Xaa = tyrosine, alanine, phenylalanine, aspartic acid, or glutamic acid.
  MISC_FEATURE (60)..(60): Xaa = tyrosine, alanine, phenylalanine, aspartic acid, or glutamic acid.
FKWYSHSKEKIQNLSLISLANLPPSGLANAVAEGIRSEENIXTIEENVXEVEEPNEXXCXVSSRQQPS

SEQ ID NO: 38 - protein, 67 aa, Artificial Sequence
  Sequence of a TIM-3 cytoplasmic domain
LKWYSYKKKKLSSLSLITLANLPPGGLANAGAVRIRSEENIYTIEENVYEVENSNEYYCYVNSQQPS

SEQ ID NO: 39 - protein, 67 aa, Artificial Sequence
  Sequence of a TIM-3 cytoplasmic domain
LKWYSSKKKKLQDLSLITLANSPPGGLVNAGAGRIRSEENIYTIEENIYEMENSNEYYCYVSSQQPS

SEQ ID NO: 40 - protein, 17 aa, Artificial Sequence
  Sequence of a TIM-3 cytoplasmic domain
SEENIYTIEENVYEVEE

(19) United States
(12) Patent Application Publication
(10) Pub. No.: US 2024/0122980 A1
(43) Pub. Date: Apr. 18, 2024

(54) USE OF TIM-3 CYTOPLASMIC TAIL IN CHIMERIC ANTIGEN RECEPTORS

(71) Applicant: University of Pittsburgh - Of the Commonwealth System of Higher Education, Pittsburgh, PA (US)

(72) Inventors: Lawrence Patrick Kane, Pittsburgh, PA (US); Jason Lohmueller, Pittsburgh, PA (US)

(73) Assignee: University of Pittsburgh - Of the Commonwealth System of Higher Education, Pittsburgh, PA (US)

(21) Appl. No.: 17/769,583

(22) PCT Filed: Oct. 16, 2020
(86) PCT No.: PCT/US2020/056074

§ 371 Date: Apr. 15, 2022

Related U.S. Application Data
(60) Provisional application No. 62/923,201, filed on Oct. 18, 2019.

(51) Int. Cl. A61K 35/17; A61K 39/00; C07K 14/705; C07K 16/28; C12N 15/86

(52) U.S. Cl. CPC A61K 35/17; A61K 39/4631; C07K 14/70517; C07K 16/2887; C12N 15/86; A61K 2239/21; A61K 2239/48; C07K 2317/622

(57) ABSTRACT

Disclosed herein are chimeric antigen receptors (CARs) that include (a) an extracellular scFv comprising a light chain variable domain (V_{L}) a heavy chain variable domain (V_{H}), wherein the scFv specifically binds to an antigen of interest; (b) a CD8 hinge domain and transmembrane domain; and (c) a cytoplasmic domain comprising (i) a TIM-3 cytoplasmic domain and an intracellular signaling domain, wherein (a)-(c) are in N-terminal to C-terminal order. Also disclosed are nucleic acid molecules encoding these CARs, host cells transformed with these nucleic acids, and the use of these compositions for treating a subject, such as a subject with a tumor.

### CROSS REFERENCE TO RELATED APPLICATIONS

This claims the benefit of U.S. Provisional Application No. 62/923,201, filed Oct. 18, 2019, which is incorporated by reference herein.

### STATEMENT OF GOVERNMENT SUPPORT

This invention was made with Government support under grant nos. AI138504 and CA206517 by the National Institutes of Health. The government has certain rights in the invention.

### FIELD OF THE DISCLOSURE

This relates to the field of chimeric antigen receptors, specifically to chimeric antigen receptors that include a T cell (or transmembrane) immunoglobulin and mucin domain-containing molecule 3 (TIM-3) cytoplasmic domain.

### BACKGROUND

T cell activation and effector function are regulated by a complex series of cell-cell interactions between a T cell antigen presenting cells (APCs). The most important interaction is between T cell receptor for antigen (TCR) and major histocompatibility complex (MHC) proteins, which present peptide antigens to the T cell. Recognition of peptide/MHC by a TCR is translated into biochemical signaling events by the TCR-associated CD3δ, ε and γ signaling chains (Courtney et al., 2018, Trends Biochem Sci 43: 108-123; Gaud et al., 2018 Nat Rev Immunol 18: 485-497). While these events are important for T cell activation, there are numerous other receptor-ligand interactions that also regulate—either positively or negatively—the ultimate effects on T cell activation and function. The negative regulators in particular have garnered much attention in recent years, and blocking antibodies to some of these (CTLA-4 and PD-1) are now clinically approved for use in selected solid tumors (Hargadon et al., 2018, Int Immunopharmacol 62: 29-39; Ribas and Wolchok, 2018, Science 359: 1350-1355). In a parallel series of studies, the use of engineered T cells expressing chimeric antigen receptors (CARs) directed at specific tumor antigens has gained significant traction in the treatment of specific hematological malignancies (June and Sadelain, 2018, N Engl J Med 379: 64-7). The efficacy of the latter approach is also regulated by both the expression of endogenous positive and negative regulators in the CAR T cells, as well as the inclusion of different co-stimulatory signaling domains in the CAR itself. A need remains for additional CARs, that can be used to target tumor cells.

### SUMMARY OF THE DISCLOSURE

Disclosed herein are CARs that include (a) an extracellular scFv comprising a light chain variable domain (V_{L}) a heavy chain variable domain (V_{H}), wherein the scFv specifically binds to an antigen of interest; (b) a CD8 hinge domain and transmembrane domain; and (c) a cytoplasmic domain comprising (i) a TIM-3 cytoplasmic domain and an intracellular signaling domain, wherein (a)-(c) are in N-terminal to C-terminal order. Also disclosed are nucleic acid molecules encoding these CARs, host cells transformed with these nucleic acids, and compositions including a therapeutically effective amount of these nucleic acid molecules or host cells. In some non-limiting examples, the host cells can be T cells.

In other embodiments, disclosed is the use of these compositions for treating a subject, such as a subject with a tumor. In specific nonlimiting examples, the scFv specifically binds a tumor antigen. In specific non-limiting examples, the tumor antigen is CD19 or CD20.

The foregoing and other features and advantages of the invention will become more apparent from the following detailed description of several embodiments which proceeds with reference to the accompanying figures.

### BRIEF DESCRIPTION OF THE FIGURES

FIG. 1: Diagram of the CAR proteins and lentiviral expression constructs for CARs containing the TIM-3 co-signaling domain or positive control CD28 co-signaling domain.

FIG. 2: Expression levels of CD28- and TIM-3-containing CARs show equivalent CAR expression. CAR expression was evaluated by antibody staining and flow cytometry for the T2A tag fused to the CAR.

FIG. 3: TIM-3 and CD28 CARs induce antigen-specific T cell marker expression to comparable levels on Jurkat CAR T cells. CAR T cells were co-incubated with the indicated target cells for 18hrs and evaluated by flow cytometry for activation marker expression (CD69, CD62L, and CD25).

FIG. 4: TIM-3 and CD28 CARs induce antigen-specific T cell marker expression to comparable levels on Primary CAR T cells. Human peripheral blood CAR T cells were co-incubated with the indicated target cells for 18hrs and evaluated by flow cytometry for activation marker expression (CD69, CD62L, and CD25).

FIG. 5: Anti-CD20 CARs with the TIM-3 co-signaling domain expressed on primary T cells mediate antigen-specific CD107a expression to similar levels of CARs with traditional co-signaling domains. The indicated CAR T cells were co-incubated with the indicated target cells for 18hrs and evaluated by antibody staining and flow cytometry for T cell activation marker expression.

FIG. 6: CARs containing signaling domains of CD28 or TIM-3 are recruited to the immune synapse. Control (top panels) Jurkat T cells or those transduced with anti-CD20 CART-CD28 (middle) or CART-TIM-3 (bottom) were mixed with Raji B cells. Cells were fixed and stained for CD3, CD20 and the CAR and imaged on an AMNIS® ImageStream as described in Methods.

FIG. 7: TIM-3 and CD28 CARs expressed on Jurkat and primary T cells mediate antigen-specific pS6 expression to similar levels. The indicated CAR T cells were co-incubated with the indicated target cells for 18hrs and evaluated by antibody staining and flow cytometry for pS6 expression.

FIG. 8: Expression levels of CARs tested. TIM-3-containing CARs. CAR expression was evaluated by antibody staining and flow cytometry for the T2A tag fused to the CAR.

FIG. 9: Function of a CD19-targeted CAR containing the cytoplasmic signaling domain of TIM-3. Primary T cells transduced with the indicated constructs were mixed with target cells—either Raji cells (which express endogenous CD19 and CD20) or with Jurkat cells stably transfected with CD19 or CD20. CD69, CD62L and CD107a levels on the surface of the CAR T cells were measured by flow cytometry after 18 hrs of co-culture.

FIG. 10: Domain structure of full-length and truncated (Tl and T2) TIM-3.

FIGS. 11A-11D. Recruitment of Tim-3 to the immune synapse of Jurkat and P14 Tg T cells. (A) Jurkat T cells transfected with Flag-tagged Tim-3 were mixed with Raji cells, either pulsed with SEE or un-pulsed, at a ratio of 1:1 for 20 mins and then were analyzed by IMAGESTREAM®. (B) Conjugates were incubated for the indicated times. The percentage of either CD3 or Tim-3 recruitment was quantified from the ratio of the intensity of either CD3 or Tim-3 within the interface mask of conjugates containing one Jurkat T cell and Raji cell to that of the whole cell mask, using IDEAS® software. (C-D) CD8⁺ T cells from control P14 TCR Tg mice or those expressing Cre-inducible Tim-3 were mixed with purified B cells from the same mice, with or without gp33 peptide, and conjugates were analyzed as above.

FIGS. 12A-12F. Neither the IgV domain nor the cytoplasmic tail is required for Tim-3 recruitment to the IS. (A) Representative images of Jurkat T cells transfected with either Flag-tagged WT Tim-3 or partial cytoplasmic truncations (T1, T2) or delta(Δ)IgV constructs and mixed with Raji cells, either pulsed with SEE or un-pulsed, at a ratio of 1:1 for 20 minutes. (B) Representative dot plots analyzing the ratio of the intensity of WT or mutant Tim-3 constructs in the interface mask between a transfected Jurkat T cell and Raji cell to that of the whole cell mask. (C) Quantification of % Tim-3 recruitment. (D) Representative images of Jurkat T cells transfected with either Flag-tagged WT or ΔCyto Tim-3 and mixed with Raji cells, either pulsed with SEE or un-pulsed, at a ratio of 1:1 for 20mins. (E) Representative dot plots analyzing the ratio of the intensity of WT or ΔCyto Tim-3 in the interface mask between a transfected Jurkat T cell and Raji cell to that of the whole cell mask. (F) Quantification of % Tim-3 recruitment. Data in each panel are representative of at least two experiments. Data shown represent the mean±SEM. **P<0.01, ***P<0.001 vs.−SEE, Tukey's multiple comparisons.

FIGS. 13A-13D. Role of the transmembrane domain in Tim-3 localization and function during IS formation. (A) Representative images of pS6 in Jurkat T cells transfected with either Flag-tagged WT or CD71tm Tim-3 and mixed with Raji cells either pulsed with SEE or un-pulsed, at a ratio of 1:1, for 20mins. (B) Quantification of CD3 (left) and Tim-3 (right) enrichment in the synapses of multiple Jurkat-Raji conjugates, with or without SEE. (C) Left—representative histogram analyzing pS6 intensity in transfected Jurkat T cells in conjugates, with or without SEE. Right—quantification of pS6 MFI across multiple conjugates. (D) Left—pS6 was analyzed within control, WT Tim-3 or Tim-3 CD71tm-transfected Jurkat T cells, stimulated with αTCR for 30 mins. Right—quantification of % pS⁶⁺ cells. Each panel is representative of at least two independent experiments. Data shown represent the mean±SEM. *P<0.05, ***P<0.001, ****P<0.0001 vs. —SEE, **P<0.01 Tukey's multiple comparisons test.

FIGS. 14A-14B. Lipid bilayer imaging of immune synapses with WT vs. CD71tm Tim-3. (A) Representative images of murine CD8⁺ T cells expressing FAP-tagged WT Tim-3 (top rows) or CD71tm Tim-3 (bottom rows). Tim-3 localization is based on FAP staining after addition of dye. TCR/CD3 localization is tracked by a labeled anti-TCR (H57) Fab fragment. Images were acquired at 15 minutes after addition of T cells to the bilayer. (B) Quantitation of average Tim-3 and CD3 synapse diameter from 50-60 cells (Tim-3) or 80-90 cells (CD3). Statistical significance was derived from student's t tests of WT vs. CD71tm.

FIGS. 15A-15D. Enforced immune synapse localization of the human Tim-3 cytoplasmic tail supports T cell activation through a chimeric antigen receptor. (A) IMAGESTREAM® analysis of immune synapse formation by the CD28-ζ or Tim3-ζ CAR, expressed in Jurkat T cells and mixed with Raji B cells. (B) Peripheral blood T cells (right) were assayed by intracellular flow cytometry for phosphorylation of ribosomal protein S6 after stimulation for 30mins with the indicated target cells. (C-D) Jurkat T cells (left) or human peripheral blood T cells (right) expressing either the CD28 or Tim-3 CAR were stimulated with the indicated target cells (see key for panel B) and analyzed by flow cytometry after 18hrs for cell-surface expression of CD69 (C) or CD25 (D). Data shown represent the mean±SEM. *P<0.05, **P<0.01, ***P<0.001, Tukey's multiple comparisons test. All stats indicate comparison with no-target conditions.

### SEQUENCES

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file [Sequence_Listing, Oct. 16, 2020, 0.0428 in bytes], which is incorporated by reference herein. In the accompanying sequence listing:

SEQ ID NO: 1 is an exemplary amino acid sequence of a linker.

SEQ ID NO: 2 is an exemplary amino acid sequence of an scFV that specifically binds CD19.

SEQ ID NO: 3 is an exemplary amino acid sequence of an scFV that specifically binds CD20.

SEQ ID NO: 4 is an exemplary amino acid sequence of a signal peptide sequence is the mouse immunoglobulin light chain kappa signal sequence.

SEQ ID NO: 5 is an exemplary amino acid sequence of a signal peptide sequence is a human granulocyte-macrophage colony-stimulating factor receptor sequence.

SEQ ID NO: 6 is an exemplary amino acid sequence of a spacer domain that is an immunoglobulin domain.

SEQ ID NO: 7 is an exemplary amino acid sequence of a CD8 hinge domain.

SEQ ID NO: 8 is an exemplary amino acid sequence of CD8 transmembrane domain.

SEQ ID NO: 9 is an exemplary amino acid sequence of a CD28 transmembrane domain.

SEQ ID NO: 10 is the amino acid sequence of amino acids 224-301 of human TIM-3.

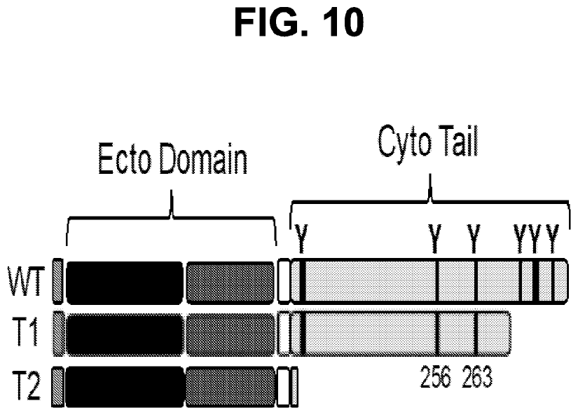

SEQ ID NO: 11 is the amino acid sequence of a TIM-3 human cytoplasmic domain lacking amino acid residues 278-301 (“truncation 1” see FIG. 10).

SEQ ID NO: 12 is the amino acid sequence of a TIM-3 human cytoplasmic domain lacking amino acid residues 264-301 (“truncation 2” see FIG. 10).

SEQ ID NO: 13 is the amino acid sequence of a variant TIM-3 human cytoplasmic domain.

SEQ ID NO: 14 is an exemplary amino acid sequence of a CD3 zeta signaling domain.

SEQ ID NO: 15 is an exemplary amino acid sequence of a CD8 signaling domain.

SEQ ID NO: 16 is an exemplary amino acid sequence of a CD28 signaling domain.

SEQ ID NO: 17 is an exemplary amino acid sequence of a CD137 signaling domain.

SEQ ID NO: 18 is an exemplary amino acid sequence of a CD137 signaling domain.

SEQ ID NO: 19 is an exemplary nucleic acid sequence of the coding region encoding pHR-EF1-aCD20-TIM-3z-T2A-TagBFP.

SEQ ID NO: 20 is an exemplary nucleic acid sequence encoding the costing region of pHR-EF1-aCD19-TIM-3z-T2A-TagBFP.

SEQ ID NO: 21 is the amino acid sequence encoded by pHR-EF1-aCD20-TIM-3z-T2A-TagBFP.

SEQ ID NO: 22 is the amino acid sequence encoded by pHR-EF1-aCD19-TIM-3z-T2A-TagBFP.

SEQ ID NOs: 23, 24, 25, 26 and 27 are the amino acid sequence of CDRs of an scFV that specifically binds CD19.

SEQ ID NOs: 28, 29, 30, 31 and 32 are the amino acid sequence of CDRs of an scFV that specifically binds CD20.

SEQ ID NO: 33 is the amino acid sequence of a 4-1BB transmembrane domain.

SEQ ID NOs: 34-40 are the amino acid sequences of TIM-3 cytoplasmic domains.

### DETAILED DESCRIPTION OF SEVERAL EMBODIMENTS

Chimeric antigen receptors (CARs) have been developed as a therapeutic for hematopoietic malignancies with defined antigens that can be targeted by engineered effector T cells. However, use of these therapies is limited in part by severe toxicity, due mainly to cytokine release syndrome (CRS). Thus, there is a need to develop modified CARs that are effective in eradicating cancers while limiting toxicity. Unexpectedly, it was determined that TIM-3, a checkpoint inhibitor, has the ability to co-stimulate T cell activation when the cytoplasmic domain of TIM-3 was included in a CAR. The disclosed CARs also have more sustained in vivo activation. In some embodiments, these CARs are less toxic than other CARs.

The protein TIM-3 was originally described as a marker for Th1 helper T cells (Monney et al., 2002, Nature 415: 536-54). Subsequent studies revealed that TIM-3 is also expressed on acutely activated CD8+ T cells and a subset of regulatory T cells, in addition to various non-T cells, including some macrophages, dendritic cells, NK cells and mast cells (Gorman and Colgan, 2014, Immunol Res 59: 56-65; Anderson et al., 2016, Immunity 44: 989-1004; Banerjee and Kane, 2018, F1000Res 7: 316). TIM-3 is highly expressed on “exhausted” CD8+ T cells observed under conditions of chronic infection or within the tumor microenvironment. These cells comprise a subset of the cells that expression the immune checkpoint molecule PD-1, which was previously described as the most robust marker for exhausted T cells; indeed, mAb's that interfere with PD-1 interactions with its ligands (PD-L1, PD-L2) have demonstrated efficacy in a subset of cancer patients (Hargadon, supra; Ribas, supra; Zou et al., 2016, Science translational medicine 8: 328rv324). Strikingly, T cells expressing high levels of both PD-1 and TIM-3 appear to be even more dysfunctional than those expressing PD-1 alone (Fourcade et al., 2010, J Exp Med 207: 2175-2186; Sakuishi et al., 2010, J Exp Med 207: 2187-2194). Consistent with this notion, dual blockade of PD-1 and TIM-3 has a modestly superior ability to “rescue” the function of a population of exhausted T cells (Fourcade et al., 2010, J Exp Med 207: 2175-2186; Sakuishi et al., 2010, J Exp Med 207: 2187-2194). Several antibodies directed against TIM-3 are now in clinical trials and combination therapies are being actively explored (He et al., 2018, Onco Targets Ther 11: 7005-700). One model that has emerged is that TIM-3 mainly functions as a negative regulator of T cell activation, in a manner similar to PD-1. Recent correlative evidence for this model was provided by the discovery of patients bearing germline loss-of-function mutations in TIM-3 (Gayden et al., 2018, Nat Genet 50: 1650-1657).

There has yet to emerge a clear-cut mechanism by which TIM-3 might transmit an inhibitory signal, for example through recruitment of phosphatases like PD-1, or by competition with co-stimulatory receptors, like CTLA-4. At least four ligands have been described to interact with TIM-3, including galectin-9, phosphatidylserine (PS), HMGB1 and CEACAM1 (Gorman and Colgan, 2014, Immunol Res 59: 56-65; Anderson et al., 2016, Immunity 44: 989-1004; Banerjee and Kane, 2018, F1000Res 7: 316), which are known to also interact with other and distinct receptors (He et al., 2018, Onco Targets Ther 11: 7005-7009; Su et al., 2011, Glycobiology 21: 1258-1265; Segawa and Nagata, 2015, Trends Cell Biol 25: 639-650; Venereau et al., 2016, Pharmacol Res 111: 534-544; Dankner et al., 2017, Oncoimmunology 6: e1328336), making the definition of important receptor-ligand interactions difficult at best. TIM-3 can also transmit positive, activation-promoting, signals (Anderson et al., 2007, Science 318: 1141-1143; Avery et al., 2018, Proc Natl Acad Sci U S A 115: 2455-2460; Gorman et al., 2014, J Immunol 192: 3133-3142; Lee et al., 2011, Mol Cell Biol 31: 3963-3974; Phong et al., 2015, J Exp Med 212: 2289-2304; Nakae et al., 2007, Blood 110: 2565-2568). There is also a model by which expression of TIM-3 might either promote the development of T cell exhaustion by enhancing or sustaining TCR signaling or actually represent a “last-ditch” effort by exhausted T cells to maintain some function (Avery et al., 2018, Proc Natl Acad Sci U S A 115: 2455-2460; Ferris et al., 2014, J Immunol 193: 1525-1530). Thus, the role of TIM-3 requires further clarification. Disclosed herein are CARs that include portions of TIM-3 in a different context than its in vivo role.

### Terms

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. “About” indicates within 5%, unless otherwise stated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various embodiments, the following explanations of terms are provided:

Administration: The introduction of a composition into a subject by a chosen route. Administration can be local or systemic. For example, if the chosen route is intravenous, the composition is administered by introducing the composition into a vein of the subject. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

Amino acid substitution: The replacement of one amino acid in peptide with a different amino acid.

Antibody: An immunoglobulin, antigen-binding molecule, or derivative thereof, that specifically binds and recognizes an analyte (antigen) such as IL-7Rα. The term “antibody” is used herein in the broadest sense and encompasses various antibody structures, including but not limited to monoclonal antibodies, polyclonal antibodies, multispecific antibodies (e.g., bispecific antibodies), and antibody molecules, so long as they exhibit the desired antigen-binding activity.

Non-limiting examples of antibodies and antigen binding molecules include, for example, intact immunoglobulins and variants and fragments thereof known in the art that retain binding affinity for the antigen. Examples of antigen binding molecules (also called “antigen binding fragments”) include but are not limited to Fv, Fab, Fab′, Fab′-SH, F(ab′)₂; diabodies; linear antibodies; single-chain antibody molecules (e.g. scFv); and multispecific antibodies formed from antibody fragments. Antigen binding molecules include antigen binding fragments either produced by the modification of whole antibodies or those synthesized de novo using recombinant DNA methodologies (see, e.g., Kontermann and Dubel (Ed), Antibody Engineering, Vols. 1-2, 2^{nd} Ed., Springer Press, 2010).

A single-chain antibody (scFv) is a genetically engineered molecule containing the V_{H} and V_{L} domains of one or more antibody(ies) linked by a suitable polypeptide linker as a genetically fused single chain molecule (see, for example, Bird et al., Science, 242: 423-426, 1988; Huston et al., Proc. Natl. Acad. Sci., 85: 5879-5883, 1988; Ahmad et al., Clin. Dev. Immunol., 2012, doi: 10.1155/2012/980250; Marbry, IDrugs, 13: 543-549, 2010). The intramolecular orientation of the V_{H}-domain and the V_{L}-domain in a scFv, is typically not decisive for scFvs. Thus, scFvs with both possible arrangements (V_{H}-domain-linker domain-V_{L}-domain; V_{L}-domain-linker domain-V_{H}-domain) may be used.

In a dsFv the V_{H} and V_{L} have been mutated to introduce a disulfide bond to stabilize the association of the chains. Diabodies also are included, which are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see, for example, Holliger et al., Proc. Natl. Acad. Sci., 90: 6444-6448, 1993; Poljak et al., Structure, 2: 1121-1123, 1994).

Antibodies also include genetically engineered forms such as chimeric antibodies (such as humanized murine antibodies) and heteroconjugate antibodies (such as bispecific antibodies). See also, Pierce Catalog and Handbook, 1994-1995 (Pierce Chemical Co., Rockford, IL); Kuby, J., Immunology, 3^{rd Ed}., W. H. Freeman & Co., New York, 1997.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. Antibody competition assays are known, and an exemplary competition assay is provided herein.

An antibody may have one or more binding sites. If there is more than one binding site, the binding sites may be identical to one another or may be different. For instance, a naturally occurring immunoglobulin has two identical binding sites, a single-chain antibody or Fab fragment has one binding site, while a bispecific or bifunctional antibody has two different binding sites.

Typically, a naturally occurring immunoglobulin has heavy chains and light chains interconnected by disulfide bonds. Immunoglobulin genes include the kappa, lambda, alpha, gamma, delta, epsilon and mu constant region genes, as well as the myriad immunoglobulin variable domain genes. There are two types of light chain, lambda (λ) and kappa (κ). There are five main heavy chain classes (or isotypes) which determine the functional activity of an antibody molecule: IgM, IgD, IgG, IgA and IgE.

Each heavy and light chain contains a constant region (or constant domain) and a variable region (or variable domain; see, e.g., Kindt et al. Kuby Immunology, 6^{th} ed., W. H. Freeman and Co., page 91 (2007).) In several embodiments, the V_{H} and V_{L} combine to specifically bind the antigen. In additional embodiments, only the V_{H} is required. For example, naturally occurring camelid antibodies consisting of a heavy chain only are functional and stable in the absence of light chain (see, e.g., Hamers-Casterman et al., Nature, 363: 446-448, 1993; Sheriff et al., Nat. Struct. Biol., 3: 733-736, 1996). References to “V_{H}” or “VH” refer to the variable region of an antibody heavy chain, including that of an antigen binding molecule, such as Fv, scFv, dsFv or Fab. References to “V_{L}” or “VL” refer to the variable domain of an antibody light chain, including that of an Fv, scFv, dsFv or Fab.

The V_{H} and V_{L} contain a “framework” region interrupted by three hypervariable regions, also called “complementarity-determining regions” or “CDRs” (see, e.g., Kabat et al., Sequences of Proteins of Immunological Interest, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The amino acid sequence boundaries of a given CDR can be readily determined using any of a number of well-known schemes, including those described by Kabat et al. (“Sequences of Proteins of Immunological Interest,” 5th Ed. Public Health Service, National Institutes of Health, Bethesda, MD, 1991; “Kabat” numbering scheme), Al-Lazikani et al., (JMB 273, 927-948, 1997; “Chothia” numbering scheme), and Lefranc et al. (“IMGT unique numbering for immunoglobulin and T cell receptor variable domains and Ig superfamily V-like domains,” Dev. Comp. Immunol., 27: 55-77, 2003; “IMGT” numbering scheme). The CDRs of each chain are typically referred to as CDR1, CDR2, and CDR3 (from the N-terminus to C-terminus) and are also typically identified by the chain in which the particular CDR is located. Thus, a V_{H} CDR3 is the CDR3 from the V_{H} of the antibody in which it is found, whereas a V_{L} CDRI is the CDR1 from the V_{L} of the antibody in which it is found. Light chain CDRs are sometimes referred to as LCDR1, LCDR2, and LCDR3. Heavy chain CDRs are sometimes referred to as HCDR1, HCDR2, and HCDR3.

A “monoclonal antibody” is an antibody obtained from a population of substantially homogeneous antibodies or from a single clone in a phage display, that is, the individual antibodies comprising the population are identical and/or bind the same epitope, except for possible variant antibodies, for example, containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein. In some examples monoclonal antibodies are isolated from a subject. Monoclonal antibodies can have conservative amino acid substitutions, which have substantially no effect on antigen binding or other immunoglobulin functions. (See, for example, Harlow & Lane, Antibodies, A Laboratory Manual, 2^{nd} ed. Cold Spring Harbor Publications, New York (2013).)

A “humanized” antibody or antigen binding molecule includes a human framework region and one or more CDRs from a non-human (such as a mouse, rat, or synthetic) antibody or antigen binding molecule. The non-human antibody or antigen binding molecule (also called “antigen binding fragment”) providing the CDRs is termed a “donor,” and the human antibody or antigen binding molecule providing the framework is termed an “acceptor.” In one embodiment, all the CDRs are from the donor immunoglobulin in a humanized immunoglobulin. Constant regions need not be present, but if they are, they can be substantially identical to human immunoglobulin constant regions, such as at least about 85-90%, such as about 95% or more identical. Hence, all parts of a humanized antibody or antigen binding fragment thereof (an “antigen binding molecule”), except possibly the CDRs, are substantially identical to corresponding parts of natural human antibody sequences.

A “chimeric antibody” is an antibody which includes sequences derived from two different antibodies, and are typically of different species. In some examples, a chimeric antibody includes one or more CDRs and/or framework regions from one human antibody and CDRs and/or framework regions from another human antibody. In other embodiments, a chimeric antibody can include the VH and VL regions of a mouse monoclonal antibody (such as the 4A10 or 2B8 antibody) and human constant regions, such as human IgG1 regions.

A “fully human antibody” or “human antibody” is an antibody, which includes sequences from (or derived from) the human genome, and does not include sequence from another species. In some embodiments, a human antibody includes CDRs, framework regions, and (if present) an Fc region from (or derived from) the human genome. Human antibodies can be identified and isolated using technologies for creating antibodies based on sequences derived from the human genome, for example by phage display or using transgenic animals (see, e.g., Barbas et al. Phage display: A Laboratory Manuel. 1^{st} Ed. New York: Cold Spring Harbor Laboratory Press, 2004. Print .; Lonberg, Nat. Biotech., 23: 1117-1125, 2005; Lonenberg, Curr. Opin. Immunol., 20: 450-459, 2008).

Biological sample: A sample obtained from a subject. Biological samples include all clinical samples useful for detection of disease (for example, a tumor) in subjects, including, but not limited to, cells, tissues, and bodily fluids, such as blood, derivatives and fractions of blood (such as serum), cerebrospinal fluid; as well as biopsied or surgically removed tissue, for example tissues that are unfixed, frozen, or fixed in formalin or paraffin. A biological sample can include T cells and/or NK cells, and can be used to generate cells of use in treatment methods. CD3 (Cluster of differentiation 3 T cell Co-receptor): A specific protein complex including at least four polypeptide chains, which are non-covalently associated with the T cell receptors on the surface of T cells. The four polypeptide chains include two CD3-epsilon chains, a CD3-delta chain and a CD3-gamma chain. CD3 is present on both helper T cells and cytotoxic T cells. Chemotherapeutic agent: Any chemical agent with therapeutic usefulness in the treatment of diseases characterized by abnormal cell growth. For example, chemotherapeutic agents can be useful for the treatment of cancer, such as T-ALL or B-ALL. Particular examples of chemotherapeutic agents that can be used include microtubule binding agents, DNA intercalators or cross-linkers, DNA synthesis inhibitors, DNA and RNA transcription inhibitors, antibodies, enzymes, enzyme inhibitors, gene regulators, and angiogenesis inhibitors. In one embodiment, a chemotherapeutic agent is a radioactive compound. One of skill in the art can readily identify a chemotherapeutic agent of use (see for example, Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., ©2000 Churchill Livingstone, Inc; Baltzer, L., Berkery, R. (eds): jOncology Pocket Guide to Chemotherapy, 2nd ed. St. Louis, Mosby-Year Book, 1995; Fischer, D. S., Knobf, M. F., Durivage, H. J. (eds): The Cancer Chemotherapy Handbook, 4th ed. St. Louis, Mosby-Year Book, 1993; Chabner and Longo, Cancer Chemotherapy and Biotherapy: Principles and Practice (4th ed.). Philadelphia: Lippincott Willians & Wilkins, 2005; Skeel, Handbook of Cancer Chemotherapy (6th ed.). Lippincott Williams & Wilkins, 2003). Combination chemotherapy is the administration of more than one agent to treat cancer.

Chimeric Antigen Receptor (CAR): An engineered T cell receptor having an extracellular antibody-derived targeting domain (such as an scFv) joined to one or more intracellular signaling domains of a T cell receptor. Generally, the disclosed CARs include at least (a) an extracellular scFv comprising a light chain variable domain (V_{L}) a heavy chain variable domain (V_{H}), wherein the scFv specifically binds to an antigen of interest; (b) hinge domain; (c) a transmembrane domain; and (d) a cytoplasmic domain comprising (i) a TIM-3 cytoplasmic domain and an intracellular signaling domain. The “hinge domain” is a structural domain in a CAR that, when the CAR is expressed in a cell, is placed between the extracellular scFv and the cell's outer membrane. The hinge domain enhances the flexibility of the scFv, reducing the spatial constraints between the CAR and its target antigen. This promotes antigen binding and synapse formation between the CAR-T cells and target cells. Hinge sequences are often membrane-proximal regions from other immune molecules including IgG, CD8, and CD28. The “transmembrane domain” is a structural component that has a hydrophobic alpha helix that spans the cell membrane. The transmembrane domain anchors the CAR to the plasma membrane, bridging the extracellular hinge and antigen recognition domains with the intracellular signaling region. The “cytoplasmic domain” includes a TIM-3 cytoplasmic domain and an intracellular signaling domain, wherein after an antigen is bound to the scFv, CAR receptors cluster together and transmit an activation signal inside a T cell. The intracellular region can include one or more domains from co-stimulatory proteins. In some embodiments the CARs contain the CD3ζ chain domain as the intracellular signaling domain, which is the primary transmitter of T cell activation signals. Second generation CARs add a co-stimulatory domain, like CD28 or 4-1BB. Third generation CARs include multiple co-stimulatory domains. Fourth generation CARs (also known as TRUCKs or armored CARs) further include factors that enhance T cell expansion, persistence, and anti-tumoral activity. This can include cytokines, such is IL-2, IL-5, IL-12 and co-stimulatory ligands. All of these types are encompassed by the present disclosure.

A “chimeric antigen receptor T cell” is a T cell expressing a CAR, and has antigen specificity determined by the antibody-derived targeting domain of the CAR. Methods of making CARs are available (see, e.g., Park et al., Trends Biotechnol., 29: 550-557, 2011; Grupp et al., N Engl J Med., 368: 1509-1518, 2013; Han et al., J. Hematol Oncol., 6: 47, 2013; PCT Publication Nos. WO2012/079000, WO2013/059593; and U.S. Publication No. 2012/0213783, each of which is incorporated by reference herein in its entirety.)

Codon Optimized: A nucleic acid molecule encoding a protein can be codon optimized for expression of the protein in a particular organism by including the codon most likely to encode a particular amino acid with the amino acid sequence. Codon usage bias is the differences in the frequency of occurrence of synonymous codons (encoding the same amino acid) in coding DNA. A codon is a series of three nucleotides (a triplet) that encodes a specific amino acid residue in a polypeptide chain or for the termination of translation. There are 20 different naturally occurring amino acids, but 64 different codons (61 codons encoding for amino acids plus 3 stop codons). Thus, there is degeneracy because one amino acid can be encoded by more than one codon. A nucleic acid sequence can be optimized for expression in a particular organism (such as a human) by evaluating the codon usage bias in that organism and selecting the codon most likely to encode a particular amino acid. Multivariate statistical methods, such as correspondence analysis and principal component analysis, are widely used to analyze variations in codon usage. Computer programs are available to implement the statistical analyses related to codon usage, such as Codon W, GCUA, and INCA.

Conditions sufficient to form an immune complex: Conditions which allow an antibody or antigen binding molecule thereof to bind to its cognate epitope to a detectably greater degree than, and/or to the substantial exclusion of, binding to substantially all other epitopes. Conditions sufficient to form an immune complex are dependent upon the format of the binding reaction and typically are those utilized in immunoassay protocols or those conditions encountered in vivo. See Harlow & Lane (Antibodies, A Laboratory Manual, 2^{nd} ed. Cold Spring Harbor Publications, New York, 2013) for a description of immunoassay formats and conditions. The conditions employed in the methods are “physiological conditions” which include reference to conditions (e.g., temperature, osmolarity, pH) that are typical inside a living mammal or a mammalian cell. While it is recognized that some organs are subject to extreme conditions, the intra-organismal and intracellular environment normally lies around pH 7 (e.g., from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

The formation of an immune complex can be detected through conventional methods known to the skilled artisan, for instance immunohistochemistry, immunoprecipitation, flow cytometry, immunofluorescence microscopy, ELISA, immunoblotting (for example, Western blot), magnetic resonance imaging, CT scans, X-ray and affinity chromatography. Immunological binding properties of selected antibodies may be quantified using methods well known in the art.

Conservative variants: “Conservative” amino acid substitutions are those substitutions that do not substantially affect or decrease a function of a protein, such as the ability of the protein to interact with a target protein. For example, an IL-7Rα-specific antibody can include up to 1, 2, 3, 4, 5, 6, 7, 8, 9, or up to 10 conservative substitutions compared to a reference antibody sequence and retain specific binding activity for IL-7Rα. The term conservative variation also includes the use of a substituted amino acid in place of an unsubstituted parent amino acid.

Furthermore, one of ordinary skill will recognize that individual substitutions, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

Conservative amino acid substitution tables providing functionally similar amino acids are well known to one of ordinary skill in the art. The following six groups are examples of amino acids that are considered to be conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

Non-conservative substitutions are those that reduce an activity or function of the IL-7Rα-specific antibody, such as the ability to specifically bind to IL-7Rα. For instance, if an amino acid residue is essential for a function of the protein, even an otherwise conservative substitution may disrupt that activity. Thus, a conservative substitution does not alter the basic function of a protein of interest.

Contacting: Placement in direct physical association; includes both in solid and liquid form, which can take place either in vivo or in vitro. Contacting includes contact between one molecule and another molecule, for example the amino acid on the surface of one polypeptide, such as an antigen, that contacts another polypeptide, such as an antibody. Contacting can also include contacting a cell for example by placing an antibody in direct physical association with a cell.

Control: A reference standard. In some embodiments, the control is a negative control, such as sample obtained from a healthy patient that does not have a particular malignancy. In other embodiments, the control is a positive control, such as a tissue sample obtained from a patient diagnosed with the malignancy. In still other embodiments, the control is a historical control or standard reference value or range of values (such as a previously tested control sample, such as a group of patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, or at least about 500%

Degenerate variant: In the context of the present disclosure, a “degenerate variant” refers to a polynucleotide encoding a protein (for example, a CAR that specifically recognizes a tumor antigen) that includes a sequence that is degenerate as a result of the genetic code. There are twenty natural amino acids, most of which are specified by more than one codon. Therefore, all degenerate nucleotide sequences are included as long as the amino acid sequence of the CAR encoded by the nucleotide sequence is unchanged.

Detectable marker: A detectable molecule (also known as a label) that is conjugated directly or indirectly to a second molecule, such as an antibody, to facilitate detection of the second molecule. For example, the detectable marker can be capable of detection by ELISA, spectrophotometry, flow cytometry, microscopy or diagnostic imaging techniques (such as CT scans, MRIs, ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). In one example, a “labeled antibody” refers to incorporation of another molecule in the antibody. For example, the label is a detectable marker, such as the incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionuclides (such as ³⁵S or ¹³¹I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels are attached by spacer arms of various lengths to reduce potential steric hindrance. Methods for using detectable markers and guidance in the choice of detectable markers appropriate for various purposes are discussed for example in Sambrook et al. (Molecular Cloning: A Laboratory Manual, 4^{th} ed, Cold Spring Harbor, New York, 2012) and Ausubel et al. (In Current Protocols in Molecular Biology, John Wiley & Sons, New York, through supplement 104, 2013).

Detecting: To identify the existence, presence, or fact of something, such as the existence of a malignancy, such as a tumor. General methods of detecting are known to the skilled artisan and may be supplemented with the protocols and reagents disclosed herein.

Expression: Transcription or translation of a nucleic acid sequence. For example, a gene can be expressed when its DNA is transcribed into an RNA or RNA fragment, which in some examples is processed to become mRNA. A gene may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. Regulation of expression can include controls on transcription, translation, RNA transport and processing, degradation of intermediary molecules such as mRNA, or through activation, inactivation, compartmentalization or degradation of specific protein molecules after they are produced.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153: 516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat;

the adenovirus late promoter; the vaccinia virus 7.5K promoter) can be used. Promoters produced by recombinant DNA or synthetic techniques may also be used to provide for transcription of the nucleic acid sequences.

A polynucleotide can be inserted into an expression vector that contains a promoter sequence, which facilitates the efficient transcription of the inserted genetic sequence of the host. The expression vector typically contains an origin of replication, a promoter, as well as specific nucleic acid sequences that allow phenotypic selection of the transformed cells.

Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

Fc polypeptide: The polypeptide including the constant region of an antibody excluding the first constant region immunoglobulin domain. Fc region generally refers to the last two constant region immunoglobulin domains of IgA, IgD, and IgG, and the last three constant region immunoglobulin domains of IgE and IgM. An Fc region may also include part or all of the flexible hinge N-terminal to these domains. For IgA and IgM, an Fc region may or may not include the tailpiece, and may or may not be bound by the J chain. For IgG, the Fc region includes immunoglobulin domains Cgamma2 and Cgamma3 (Cγ2 and Cγ3) and the lower part of the hinge between Cgamma1 (Cγ1) and Cγ2. Although the boundaries of the Fc region may vary, the human IgG heavy chain Fc region is usually defined to include residues C226 or P230 to its carboxyl-terminus, wherein the numbering is according to the EU index as in Kabat. For IgA, the Fc region includes immunoglobulin domains Calpha2 and Calpha3 (Cα2 and Cα3) and the lower part of the hinge between Calpha1 (Cα1) and Cα2.

Isolated: A biological component (such as a nucleic acid, peptide, protein or protein complex, for example an antibody or a cell) that has been substantially separated, produced apart from, or purified away from other biological components and cells of the organism in which the component naturally occurs, that is, other chromosomal and extra-chromosomal DNA and RNA, and proteins. Thus, isolated nucleic acids, peptides proteins and cells include those purified by standard purification methods. The term also embraces nucleic acids, peptides and proteins prepared by recombinant expression in a host cell, as well as, chemically synthesized nucleic acids.

An isolated nucleic acid molecule, peptide or protein, for example an antibody, or a cell, can be at least 50%, at least 60%, at least 70%, at least 80%, at least 90%, at least 95%, at least 96%, at least 97%, at least 98%, or at least 99% pure.

Linker: A bi-functional molecule that can be used to link two molecules into one contiguous molecule, for example, to link an effector molecule to an antibody. In some embodiments, the provided conjugates include a linker between the effector molecule or detectable marker and an antibody. In some cases, a linker is a peptide within an antigen binding molecule (such as an Fv fragment or an scFv fragment) which serves to indirectly bond the V_{H} and V_{L}.

The terms “conjugating,” “joining,” “bonding,” or “linking” can refer to making two molecules into one contiguous molecule; for example, linking two polypeptides into one contiguous polypeptide, or covalently attaching an effector molecule or detectable marker radionuclide or other molecule to a polypeptide, such as an scFv. In the specific context, the terms include reference to joining a ligand, such as an antibody moiety, to an effector molecule. The linkage can be either by chemical or recombinant means. “Chemical means” refers to a reaction between the antibody moiety and the effector molecule such that there is a covalent bond formed between the two molecules to form one molecule.

Lymphoid Malignancy: Lymphoma and leukemia. Hematological malignancies may derive from either of the two major blood cell lineages: myeloid and lymphoid cell lines. The myeloid cell line includes granulocytes, erythrocytes, thrombocytes, macrophages and mast cells; the lymphoid cell line produces B, T, NK and plasma cells. Lymphoma, lymphocytic leukemia, and myeloma are from the lymphoid line.

Natural Killer (NK) cells: NK cells are innate lymphoid cells that are large granular lymphocytes (LGL) and are differentiated from the common lymphoid progenitor-generating B and T lymphocytes. NK cells are known to differentiate and mature in the bone marrow, lymph nodes, spleen, tonsils, and thymus. NK cells do not express T-cell antigen receptors (TCR) or pan T marker CD3 or surface immunoglobulins (Ig) B cell receptors, but they usually express the surface markers CD16 (FcγRIII) and CD56 in humans, NK1.1 or NK1.2 in C57BL/6 mice. NKp46 cell surface marker is expressed in humans, several strains of mice and in monkey species.

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The nucleotides can be ribonucleotides, deoxyribonucleotides, or modified forms of either nucleotide. The term includes single- and double-stranded forms of DNA.

Operably linked: A first nucleic acid sequence is operably linked with a second nucleic acid sequence when the first nucleic acid sequence is placed in a functional relationship with the second nucleic acid sequence. For instance, a promoter, such as the CMV promoter, is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers of use are conventional. Remington's Pharmaceutical Sciences, by E. W. Martin, Mack Publishing Co., Easton, PA, 19th Edition, 1995, describes compositions and formulations suitable for pharmaceutical delivery of the disclosed agents.

In general, the nature of the carrier will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, added preservatives (such as on-natural preservatives), and pH buffering agents and the like, for example sodium acetate or sorbitan monolaurate. In particular examples, the pharmaceutically acceptable carrier is sterile and suitable for parenteral administration to a subject for example, by injection. In some embodiments, the active agent and pharmaceutically acceptable carrier are provided in a unit dosage form such as a pill or in a selected quantity in a vial. Unit dosage forms can include one dosage or multiple dosages (for example, in a vial from which metered dosages of the agents can selectively be dispensed).

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein are intended to encompass any amino acid sequence and include modified sequences such as glycoproteins. A polypeptide includes both naturally occurring proteins, as well as those that are recombinantly or synthetically produced. A polypeptide has an amino terminal (N-terminal) end and a carboxy-terminal end. In some embodiments, the polypeptide is a disclosed CAR.

Polypeptide modifications: Polypeptides can be modified by a variety of chemical techniques to produce derivatives having essentially the same activity and conformation as the unmodified peptides, and optionally having other desirable properties. For example, carboxylic acid groups of the protein, whether carboxyl-terminal or side chain, may be provided in the form of a salt of a pharmaceutically-acceptable cation or esterified to form a C₁-C₁₆ ester, or converted to an amide of formula NR₁R₂ wherein R₁ and R₂ are each independently H or C₁-C₁₆ alkyl, or combined to form a heterocyclic ring, such as a 5- or 6-membered ring. Amino groups of the peptide, whether amino-terminal or side chain, may be in the form of a pharmaceutically-acceptable acid addition salt, such as the HCl, HBr, acetic, benzoic, toluene sulfonic, maleic, tartaric and other organic salts, or may be modified to C₁-C₁₆ alkyl or dialkyl amino or further converted to an amide.

Hydroxyl groups of the peptide side chains can be converted to C₁-C₁₆ alkoxy or to a C₁-C₁₆ ester using well-recognized techniques. Phenyl and phenolic rings of the peptide side chains can be substituted with one or more halogen atoms, such as F, Cl, Br or I, or with C₁-C₁₆ alkyl, C₁-C₁₆ alkoxy, carboxylic acids and esters thereof, or amides of such carboxylic acids. Methylene groups of the peptide side chains can be extended to homologous C₂-C₄ alkylenes. Thiols can be protected with any one of a number of well-recognized protecting groups, such as acetamide groups.

Recombinant: A recombinant nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques. A recombinant protein is one that has a sequence that is not naturally occurring or has a sequence that is made by an artificial combination of two otherwise separated segments of sequence. In several embodiments, a recombinant protein is encoded by a heterologous (for example, recombinant) nucleic acid that has been introduced into a host cell, such as a bacterial or eukaryotic cell. The nucleic acid can be introduced, for example, on an expression vector having signals capable of expressing the protein encoded by the introduced nucleic acid or the nucleic acid can be integrated into the host cell chromosome.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods. Methods of alignment of sequences for comparison are well known in the art. Various programs and alignment algorithms are described in: Smith and Waterman, Adv. Appl. Math. 2: 482, 1981; Needleman and Wunsch, J. Mol. Biol. 48: 443, 1970; Pearson and Lipman, Proc. Natl. Acad. Sci. U.S.A. 85: 2444, 1988; Higgins and Sharp, Gene 73: 237, 1988; Higgins and Sharp, CABIOS 5: 151, 1989; Corpet et al., Nucleic Acids Research 16: 10881, 1988; and Pearson and Lipman, Proc. Natl. Acad. Sci. U.S.A. 85: 2444, 1988. Altschul et al., Nature Genet. 6: 119, 1994, presents a detailed consideration of sequence alignment methods and homology calculations.

The NCBI Basic Local Alignment Search Tool (BLAST) (Altschul et al., J. Mol. Biol. 215: 403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Homologs and variants of a V_{L} or a V_{H} of an antibody that specifically binds a polypeptide are typically characterized by possession of at least about 75%, for example at least about 80%, 85%, 90%, 91%, 92%, 93%, 94%, 95%, 96%, 97%, 98% or 99% sequence identity counted over the full-length alignment with the amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Terms used to describe sequence relationships between two or more nucleotide sequences or amino acid sequences include “reference sequence,” “selected from,” “comparison window,” “identical,” “percentage of sequence identity,” “substantially identical,” “complementary,” and “substantially complementary.”

For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, e.g., by the local homology algorithm of Smith & Waterman, Adv. Appl. Math. 2: 482, 1981, by the homology alignment algorithm of Needleman & Wunsch, J. Mol. Biol. 48: 443, 1970, by the search for similarity method of Pearson & Lipman, Proc. Nat'l. Acad. Sci. USA 85: 2444, 1988, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Sambrook et al. (Molecular Cloning: A Laboratory Manual, 4^{th} ed, Cold Spring Harbor, New York, 2012) and Ausubel et al. (In Current Protocols in Molecular Biology, John Wiley & Sons, New York, through supplement 104, 2013). One example of a useful algorithm is PILEUP. PILEUP uses a simplification of the progressive alignment method of Feng and Doolittle, J. Mol. Evol. 35: 351-360, 1987. The method used is similar to the method described by Higgins and Sharp, CABIOS 5: 151-153, 1989. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12: 387-395, 1984.

Another example of algorithms that are suitable for determining percent sequence identity and sequence similarity are the BLAST and the BLAST 2.0 algorithm, which are described in Altschul et al., J. Mol. Biol. 215: 403-410, 1990 and Altschul et al., Nucleic Acids Res. 25: 3389-3402, 1977. Software for performing BLAST analyses is publicly available through the National Center for Biotechnology Information (ncbi.nlm.nih.gov). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see Henikoff and Henikoff, Proc. Natl. Acad. Sci. USA 89: 10915, 1989). An oligonucleotide is a linear polynucleotide sequence of up to about 100 nucleotide bases in length.

Specifically bind: When referring to an antibody or antigen binding molecule, such as an scFv, refers to a binding reaction which determines the presence of a target protein, peptide, or polysaccharide in the presence of a heterogeneous population of proteins and other biologics. Thus, under designated conditions, an antibody binds preferentially to a particular target protein, peptide or polysaccharide (such as tumor antigen) and does not bind in a significant amount to other proteins or polysaccharides present in the sample or subject. Specific binding can be determined by methods known in the art. With reference to an antibody-antigen complex, specific binding of the antigen and antibody has a K_{D} of less than about 10⁻⁷ Molar, such as less than about 10⁻⁸ Molar, 10⁻⁹, or even less than about 10⁻¹⁰ Molar.

K_{D} refers to the dissociation constant for a given interaction, such as a polypeptide ligand interaction or an antibody antigen interaction. For example, for the bimolecular interaction of an antibody or antigen binding molecule and an antigen it is the concentration of the individual components of the bimolecular interaction divided by the concentration of the complex.

The antigen binding molecule of the CARs disclosed herein specifically binds to a defined target (or multiple targets, in the case of a bispecific antibody). As one non-limiting example, an antigen binding molecule that specifically binds to an epitope on CD19 is an antibody that binds substantially to CD19, including cells or tissue expressing CD19 substrate to which the CD19 is attached, or CD19 in a biological specimen. It is, of course, recognized that a certain degree of non-specific interaction may occur between an antigen binding molecule or corresponding CAR (such as a CAR that specifically binds CD19) and a non-target (such as a cell that does not express CD19). Typically, specific binding results in a much stronger association between the antigen binding molecule and protein or cells bearing the antigen than between the antibody and protein or cells lacking the antigen. Specific binding typically results in greater than 2-fold, such as greater than 5-fold, greater than 10-fold, or greater than 100-fold increase in amount of bound antigen binding molecule (per unit time) to a protein including the epitope or cell or tissue expressing the target epitope as compared to a protein or cell or tissue lacking this epitope. Specific binding to a protein under such conditions requires an antigen binding molecule that is selected for its specificity for a particular protein. A variety of immunoassay formats are appropriate for selecting antigen binding molecules or other ligands specifically immunoreactive with a particular protein. For example, solid-phase ELISA immunoassays are routinely used to select monoclonal antibodies specifically immunoreactive with a protein. See Harlow & Lane, Antibodies, A Laboratory Manual, 2^{nd} ed., Cold Spring Harbor Publications, New York (2013), for a description of immunoassay formats and conditions that can be used to determine specific immunoreactivity.

Subject: Living multi-cellular vertebrate organisms, a category that includes human and non-human mammals. In an example, a subject is a human. In a particular example, the subject is a pediatric subject, such as a human child age 2-5 years old. In an additional example, a subject is selected that has a lymphoid malignancy or is at risk of having a lymphoid malignancy. In a further example, a subject is selected that has a solid tumor or is at risk of having a solid tumor.

T cell: A type of lymphocyte that plays a central role in cell-mediated immunity. T cells can be distinguished from other lymphocytes, such as B cells and natural killer cells, by the presence of a T-cell receptor on the cell surface. They are called T cells because they mature in the thymus from thymocytes. Generally, mature T cells express CD3.

T-cell (or transmembrane) immunoglobulin and mucin domain containing molecule (Tim) 3: A protein that in humans is encoded by the Hepatitis A virus cellular receptor (HAVCR)2 gene. Tim proteins have a similar structure, in which the extracellular region consists of membrane distal single variable immunoglobulin domain (IgV) and a glycosylated mucin domain of variable length located closer to the membrane. The intracellular domain of TIM-3 is called C-terminal cytoplasmic tail. It contains five conserved tyrosine residues that interact with multiple components of T cell receptor (TCR) complex signaling pathway (e.g. Lck and Fyn tyrosine kinases) and may negatively regulate its function. In vivo, TIM-3 acts as an immune checkpoint. Exemplary TIM-3 amino acid and nucleic acid sequences are provided in the following GENBANK® Accession Entries, which are incorporated by reference as available on October 1, 2019:

Human mRNA and protein sequences: GENBANK® Accession Nos. NM_032782.5 and NP_116171.3 (September 16, 2019), respectively.

Mouse mRNA and protein sequences: GENBANK® Accession Nos. NM_134250.2 and NP_599011.2, respectively.

Rat mRNA and protein sequences: GENBANK® Accession Nos. NM_001100762.1 and NP_001094232.1, respectively.

Therapeutically effective amount: The amount of an agent (such as a T cells and/or NK cells expressing a CAR) that alone, or together with one or more additional agents, induces the desired response, such as, for example treatment of a tumor antigen-positive cancer, in a subject. When administered to a subject, a dosage will generally be used that will achieve target tissue concentrations that has been shown to achieve a desired in vitro effect. Ideally, a therapeutically effective amount provides a therapeutic effect without causing a substantial cytotoxic effect in the subject.

In one example, a desired response is to decrease the size, volume, or number (such as metastases) of cancer cells in a subject, and/or neoplastic lesions or number of leukemia cells in blood in a subject. For example, the agent can decrease the size, volume, or number of cancer cells, and/or neoplastic lesions or number of leukemia cells in blood by a desired amount, for example by at least 5%, at least 10%, at least 15%, at least 20%, at least 25%, at least 30%, at least 50%, at least 75%, at least 90%, or at least 95% as compared to a response in the absence of the agent.

Several preparations disclosed herein are administered in therapeutically effective amounts. A therapeutically effective amount that is administered to a human or veterinary subject will vary depending upon a number of factors associated with that subject, for example the overall health of the subject. A therapeutically effective amount can be determined by varying the dosage and measuring the resulting therapeutic response, such as the regression of a lymphoid malignancy. Therapeutically effective amounts also can be determined through various in vitro, in vivo or in situ immunoassays. The disclosed agents can be administered in a single dose, or in several doses, as needed to obtain the desired response. However, the therapeutically effective amount can be dependent on the source applied, the subject being treated, the severity and type of the condition being treated, and the manner of administration.

A therapeutically effective amount encompasses a fractional dose that contributes in combination with previous or subsequent administrations to attaining a therapeutic response. For example, a therapeutically effective amount of an agent can be administered in a single dose, or in several doses, for example daily, during a course of treatment lasting several days or weeks. However, the therapeutically effective amount can depend on the subject being treated, the severity and type of the condition being treated, and the manner of administration. A unit dosage form of the agent can be packaged in a therapeutic amount, or in multiples of the therapeutic amount, for example, in a vial (e.g., with a pierceable lid) or syringe having sterile components.

Transduced and Transformed: A transformed cell is a cell into which a nucleic acid molecule has been introduced by molecular biology techniques. As used herein, the terms transduction and transformation encompass all techniques by which a nucleic acid molecule might be introduced into such a cell, including transfection with viral vectors, the use of plasmid vectors, and introduction of DNA by electroporation, lipofection, and particle gun acceleration.

Treating or Preventing a disease: “Preventing” a disease refers to inhibiting the full development of a disease. “Treating” refers to a therapeutic intervention that ameliorates a sign or symptom of a disease or pathological condition after it has begun to develop, such as a reduction in tumor burden or a decrease in the number of size of metastases. “Ameliorating” refers to the reduction in the number or severity of signs or symptoms of a disease, such as a malignancy.

Tumor: An abnormal growth of cells, which can be benign or malignant (a malignancy). Cancer is a malignant tumor (a malignancy), which is characterized by abnormal or uncontrolled cell growth. Other features often associated with malignancy include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system.

The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.”

Examples of hematological tumors include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia. In specific non-limiting examples, the lymphoid malignancy can be adult T cell leukemia, cutaneous T cell lymphoma, anaplastic large cell lymphoma, Hodgkin's lymphoma, or a diffuse large B cell lymphoma.

Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyrgioma, ependymoma, pinealoma, hemangioblastoma, acoustic neuroma, oligodendroglioma, meningioma, melanoma, neuroblastoma and retinoblastoma). In several examples, a tumor is breast, ovarian, gastric, esophageal, skin, lung or head and neck cancer.

Tumor Antigen: An antigenic protein produced by tumor cells. Tumor antigens include tumor-specific antigens (TSA), which are present only on tumor cells and not on any other cell and tumor-associated antigens (TAA), which are present on some tumor cells and also some normal cells. Any protein produced in a tumor cell that has an abnormal structure due to mutation or to differential post-translational chemical modification can act as a tumor antigen. Additionally, TAAs include proteins that are expressed at higher levels in tumor cells versus normal cells.

Vector: A nucleic acid molecule as introduced into a host cell, thereby producing a transformed host cell. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant nucleic acid vectors having at least some nucleic acid sequences derived from one or more viruses. A replication deficient viral vector is a vector that requires complementation of one or more regions of the viral genome required for replication due to a deficiency in at least one replication-essential gene function. For example, such that the viral vector does not replicate in typical host cells, especially those in a human patient that could be infected by the viral vector in the course of a therapeutic method.

### Chimeric Antigen Receptors

Chimeric antigen receptors (CARs) have been developed as a therapeutic for hematopoietic malignancies with defined antigens that can be targeted by engineered effector T cells. However, use of these therapies is limited in part by severe toxicity, due mainly to cytokine release syndrome (CRS). Thus, there is a need to develop modified CARs that are effective in eradicating cancers while limiting toxicity. It was disclosed that, unexpectedly, that the cytoplasmic domain of TIM-3 has the ability to co-stimulate T cell activation.

Disclosed herein is a CAR that includes a) an extracellular antigen binding molecule, such as an scFv comprising a light chain variable domain (V_{L}) a heavy chain variable domain (V_{H}), wherein the scFv specifically binds to an antigen of interest; (b) a CD8 hinge domain and a CD8 transmembrane domain; and (d) a cytoplasmic domain comprising (i) a TIM-3 cytoplasmic domain and (ii) an intracellular signaling domain, wherein (a)-(c) are in N-terminal to C-terminal order. The intracellular signaling domain is heterologous to the TIM-3 cytoplasmic domain. In some embodiments, the additional intracellular co-stimulatory signaling domain is positioned a) C-terminal to the TIM-3 cytoplasmic domain or b) between the TIM-3 cytoplasmic domain and the intracellular signaling domain.

### A. Antigen Binding Molecule

The disclosed CARS include an extracellular antigen binding molecule, such as an scFV, at their amino (N) terminus. The extracellular scFv includes a light chain variable domain (V_{L}) a heavy chain variable domain (V_{H}), wherein the scFv specifically binds to an antigen of interest. In some embodiments, the antigen is a tumor antigen. In some embodiments, the tumor antigen is CD22, CD123, CEA, EGFRvIII, ErbB2, HER2, IL-13ralpha2, MUCI, CD19 or CD20. In specific non-limiting examples, the tumor antigen is CD19 or CD20.

In some embodiments, the antigen binding domain can include a V_{H} and a V_{L} including the HCDR1, HCDR2, HCDR3, LCDR1, LCDR2, and LCDR3 of the V_{H} and V_{L}, respectively, of the antibody that specifically binds CD22, CD123, CEA, EGFRVIII, ErbB2, HER2, IL-13ralpha2, MUC1, CD19 or CD20. In several embodiments, the antigen binding domain can be a scFv. In some embodiments, the CDRs can be identified by Kabat, Chothia or IMGT.

In several embodiments, the antigen binding domain can be a scFv. To create a scFv the V_{L}- and V_{H}-encoding DNA fragments can be operatively linked to another fragment encoding a flexible linker, e.g., encoding the amino acid sequence (Gly4-Ser)3, such that the V_{L} and V_{H} sequences can be expressed as a contiguous single-chain protein, with the V_{L} and V_{H} domains joined by the flexible linker (see, e.g., Bird et al., Science 242: 423-426, 1988; Huston et al., Proc. Natl. Acad. Sci. USA 85: 5879-5883, 1988; McCafferty et al., Nature 348: 552-554, 1990; Kontermann and Dubel (Ed), Antibody Engineering, Vols. 1-2, 2^{nd} Ed., Springer Press, 2010; Harlow and Lane, Antibodies: A Laboratory Manual, 2^{nd}, Cold Spring Harbor Laboratory, New York, 2013,). Optionally, a cleavage site can be included in a linker, such as a furin cleavage site. In some embodiments, the scFv includes a V_{H} and a V_{L} joined by a peptide linker, such as a linker including the amino acid sequence set forth as SSGGGGSGGGGSGGGGS (SEQ ID NO: 1). The V_{L} and V_{H} can ben in any order, such that either the V_{L} or the V_{H} is at the N-terminus of the scFv. In one specific non-limiting example, the V_{L} is at the N-terminus.

In some embodiments, the disclosed CAR includes the antigen binding domain includes a V_{H} and a V_{L} including the HCDR1, HCDR2, HCDR3, LCDR1, LCDR2, and LCDR3, as identified by Kabat, Chothia or IMGT, of the V_{H} and V_{L}, respectively of an antibody that specifically binds CD19. An exemplary antibody is provided below, in scFv format:

(SEQ ID NO: 2)
DIQMTQTTSSLSASLGDRVTISCRASQDISKYLNWYQQKP
DGTVKLLIYHTSRLHSGVPSRFSGSGSGTDYSLTISNLEQ
EDIATYFCQQGNTLPYTFGGGTKLEITGGGGSGGGGSGGG
GSEVKLQESGPGLVAPSQSLSVTCTVSGVSLPDYGVSWIR
QPPRKGLEWLGVIWGSETTYYNSALKSRLTIIKDNSKSQV
FLKMNSLQTDDTAIYYCAKHYYYGGSYAMDYWGQGTSVTV
SS

The CDRs for V_{H} are CDR1: GVSLPDYG (SEQ ID NO: 23), CDR2: IWGSETT (SEQ ID NO: 24), and CDR3: AKHYYYGGSYAMDY (SEQ ID NO: 25), and the CDRs for V_{L} are CDR1: QDISKY (SEQ ID NO: 26), CDR2: HTS, and CDR3:QQGNTLPYT (SEQ ID NO: 27).

In other embodiments, the disclosed CAR includes the antigen binding domain includes a V_{H} and a V_{L} including the HCDR1, HCDR2, HCDR3, LCDRI, LCDR2, and LCDR3, as identified by Kabat, Chothia or IMGT, of the V_{H} and V_{L}, respectively of an antibody that specifically binds CD20. An exemplary antibody is provided below, in scFv format:

(SEQ ID NO: 3)
GDIVLTQSPAILSASPGEKVTMTCRASSSVNYMDWYQKKP
GSSPKPWIYATSNLASGVPARFSGSGSGTSYSLTISRVEA
EDAATYYCQQWSFNPPTFGGGTKLEIKGSTSGGGSGGGSG
GGGSSEVQLQQSGAELVKPGASVKMSCKASGYTFTSYNMH
WVKQTPGQGLEWIGAIYPGNGDTSYNQKFKGKATLTADKS
SSTAYMQLSSLTSEDSADYYCARSNYYGSSYWFFDVWGAG
TTVTVSS

The CDRs for V_{H} are CDR1: GYTFTSYN (SEQ ID NO: 28), CDR2:IYPGNGDT (SEQ ID NO: 29), and CDR3: ARSNYYGSSYWFFDV (SEQ ID NO: 30), and the CDRs for V_{L} are CDR1:SSVNY (SEQ ID NO: 31), CDR2:ATS, and CDR3:QQWSFNPPT (SEQ ID NO: 32).

The CAR can include an antigen binding molecule, such as an scFv, that includes a V_{H} and a V_{L} including the HCDR1, HCDR2, HCDR3, LCDR1, LCDR2, and LCDR3, as identified by Kabat, Chothia or IMGT, of the V_{H} and V_{L}, respectively of any antibody that expresses a tumor antigen. In some embodiments, the tumor antigen is expressed by a hematologic tumor, such as a lymphoma or a leukemia, such as, but not limited to, CD19 and/or CD20. In other embodiments, the tumor antigen is expressed by a solid tumor. Additional exemplary tumor antigens include one or more of the following RAGE-1, tyrosinase, MAGE-1, MAGE-2, NY-ESO-1, Melan-A/MART-1, glycoprotein (gp) 75, gp100, beta-catenin, preferentially expressed antigen of melanoma (PRAME), MUM-1, Wilms tumor (WT)-1, carcinoembryonic antigen (CEA), and PR-1. Additional tumor antigens are known in the art (for example see Novellino et al., Cancer Immunol. Immunother. 54(3): 187-207, 2005) and are described below. Tumor antigens are also referred to as “cancer antigens.” The tumor antigen can be any tumor-associated antigen, which are well known in the art and include, for example, carcinoembryonic antigen (CEA), B-human chorionic gonadotropin, alphafetoprotein (AFP), lectin-reactive AFP, thyroglobulin, RAGE-1, MN-CA IX, human telomerase reverse transcriptase, RUI, RU2 (AS), intestinal carboxyl esterase, mut hsp70-2, macrophage colony stimulating factor, prostase, prostate-specific antigen (PSA), PAP, NY-ESO-1, LAGE-1a, p53, prostein, PSMA, Her2/neu, survivin and telomerase, prostate-carcinoma tumor antigen-1, MAGE, ELF2M, neutrophil elastase, ephrinB2, CD22, insulin growth factor (IGF)-I, IGF-II, IGF-I receptor and mesothelin. A list of selected tumor antigens and their associated tumors are shown below.

### Exemplary Tumors and Their Tumor Antigens

| Tumor | Tumor Associated Target Antigens |
|---|---|
| Acute myelogenous leukemia | Wilms tumor 1 (WT1), PRAME, PR1, |
| | proteinase 3, elastase, cathepsin G |
| Chronic myelogenous leukemia | WT1, PRAME, PR1, proteinase 3, |
| | elastase, cathepsin G |
| Myelodysplastic syndrome | WT1, PRAME, PR1, proteinase 3, |
| | elastase, cathepsin G |
| Acute lymphoblastic leukemia | PRAME |
| Chronic lymphocytic leukemia | Survivin |
| Non-Hodgkin's lymphoma | Survivin |
| Multiple myeloma | NY-ESO-1 |
| Malignant melanoma | MAGE, MART, Tyrosinase, PRAME |
| | GP100 |
| Breast cancer | WT1, Herceptin, epithelial tumor |
| | antigen (ETA) |
| Lung cancer | WT1 |
| Ovarian cancer | CA-125 |
| Prostate cancer | PSA |
| Pancreatic cancer | CA19-9, RCAS1 |
| Colon cancer | CEA |
| Cervical Cancer | SCC, CA125, CEA, Cytokeratins (TPA, |
| | TPS, Cyfra21-1) |
| Renal cell carcinoma (RCC) | Fibroblast growth factor 5 |
| Germ cell tumors | AFP |

The CAR can include a signal peptide sequence, e.g., N-terminal to the antigen binding domain, such as the scFv. The signal peptide sequence can include any suitable signal peptide sequence. In an embodiment, the signal peptide sequence is the mouse immunoglobulin light chain kappa signal sequence, such as an amino acid sequence including of consisting of MDFQVQIFSFLLISASVIMSRG (SEQ ID NO: 4). However, other signal sequences known in the art can be utilized. In another example, the signal peptide sequence is a human granulocyte-macrophage colony-stimulating factor (GM-CSF) receptor sequence, such as an amino acid sequence including or consisting of LLVTSLLLCELPHPAFLLIPDT (SEQ ID NO: 5). In a further example, the signal peptide sequence is an IL-2 signal peptide. While the signal peptide sequence may facilitate expression of the CAR on the surface of the cell, the presence of the signal peptide sequence in an expressed CAR is not necessary in order for the CAR to function. Upon expression of the CAR on the cell surface, the signal peptide sequence may be cleaved off of the CAR. Accordingly, in some embodiments, the CAR lacks a signal peptide sequence.

### B. Hinge Domain and Transmembrane Domaine

Between the antigen binding domain and the transmembrane domain of the CAR, there can be a hinge domain, also called a spacer domain, which includes a polypeptide sequence. The spacer domain may comprise up to 300 amino acids, such as 10 to 100 amino acids, for example 25 to 50 amino acids.

In some embodiments, the spacer domain can include an immunoglobulin domain, such as a human immunoglobulin sequence. In an embodiment, the immunoglobulin domain comprises an immunoglobulin CH2 and CH3 immunoglobulin G (IgG₁) domain sequence (CH2CH3). In this regard, the spacer domain can include an immunoglobulin domain comprising or consisting of the amino acid sequence set forth as SEQ ID NO: 6:

EPKSCDKTHTCPPCPAPELLGGPSVFLFPPKPKDTLMISR
TPEVTCVVVDVSHEDPEVKFNWYVDGVEVHNAKTKPREEQ
YNSTYRVVSVLTVLHQDWLNGKEYKCKVSNKALPAPIEKT
ISKAKGQPREPQVYTLPPSRDELTKNQVSLTCLVKGFYPS
DIAVEWESNGQPENNYKTTPPVLDSDGSFFLYSKLTVDKS
RWQQGNVFSCSVMHEALHNHYTQKSLSLSPGKKDPK

Without being bound to a particular theory, it is believed that the CH2CH3 domain extends the antigen binding domain of the CAR away from the membrane of CAR-expressing cells and may more accurately mimic the size and domain structure of a native TCR. However, the CH2CH3 domain may not be necessary.

Optionally, a short oligo- or polypeptide linker, preferably between 2 and 10 amino acids in length, called a hinge domain, may form the linkage between the transmembrane domain and the intracellular T cell signaling domain and/or T cell costimulatory domain of the CAR. An exemplary linker sequence includes one or more glycine-serine doublets.

In some embodiments, the hinge domain is a CD8 hinge domain. An exemplary hinge domain is:

(SEQ ID NO: 7)
TTTPAPRPPTPAPTIASQPLSLRPEACRPAAGGAVHTRGLDFACD.

In some embodiments, the spacer is omitted, so that it is not present.

The transmembrane domain may be derived either from a natural or from a synthetic source. Where the source is natural, the domain may be derived from any membrane-bound or transmembrane protein. Exemplary transmembrane domains for use in the disclosed CARs can include at least the transmembrane region(s) of) the alpha, beta or zeta chain of the T-cell receptor, CD28, CD3 epsilon, CD45, CD4, CD5, CD8, CD 16, CD22, CD33, CD37, CD64, CD80, CD86, CD 134, CD137, CD154. Alternatively, the transmembrane domain may be synthetic, in which case it will comprise predominantly hydrophobic residues such as leucine and valine. In several embodiments, a triplet of phenylalanine, tryptophan and valine will be found at each end of a synthetic transmembrane domain. In one non-limiting example, the hinge domain is a CD8 transmembrane domain.

In some embodiments, the transmembrane domain comprises the transmembrane domain of a T cell receptor, such as a CD8 transmembrane domain. Thus, the CAR can include a CD8 transmembrane domain including or consisting of:

(SEQ ID NO: 8)
IYIWAPLAGTCGVLLLSLVITLYC.

In further embodiments, the CAR includes both the CD8 hinge domain and the CD8 transmembrane domain.

In another embodiment, the transmembrane domain comprises the transmembrane domain of a T cell costimulatory molecule, such as CD137 or CD28. Thus, the CAR can include a CD28 transmembrane domain including or consisting of FWVLVVVGGVLACYSLLVTVAFIIFWV (SEQ ID NO: 9)

or transmembrane domain from 4-1BB:

(SEQ ID NO: 33)
IISFFLALTSTALLFLLFFLTLRFSVV.

### Cytoplasmic Domain

The cytoplasmic domain includes a TIM-3 cytoplasmic domain and an intracellular signaling domain. In some embodiments, the TIM-3 cytoplasmic domain can include, or consist of, amino acids 224-301 of human TIM-3 (UniProt identifier Q8TDQ0-1, Oct. 1, 2019, incorporated herein by reference),

(SEQ ID NO: 10)
FKWYSHSKEKIQNLSLISLANLPPSGLANAVAEGIRSEENIYTIEENVY
EVEEPNEYYCYVSSRQQPSQPLGCRFAMP.

In some embodiments, the TIM-3 cytoplasmic domain is at least about 80%, at least about 85%, at least about 90%, at least about 95%, at least about 96%, at least about 97%, at least about 98%, or at least about 99% identical to SEQ ID NO: 10.

Similar regions can be used from the rat or mouse TIM-3 amino acid sequence, see the GENBANK® Accession numbers listed above.

In further embodiments, amino acids are deleted from the C-terminal end of SEQ ID NO: 10, such as at most 55, 50, 45, 40, 35, 30, 25, 20, 15, or 5 amino acids are removed. In more embodiments the deletion includes the three C-terminal tyrosines (Y) of SEQ ID NO: 10. In more embodiments, the TIM-3 human cytoplasmic domain comprises or consists of SEQ ID NO: 10 lacking amino acid residues 278-301 (“truncation 1” see FIG. 10):

(SEQ ID NO: 11)
FKWYSHSKEKIQNLSLISLANLPPSGLANAVAEGIRSEENIYTIEENVY
EVEEP

In another embodiment, the TIM-3 human cytoplasmic domain comprises or consists of:

(SEQ ID NO: 34)
FKWYSHSKEKIQNLSLISLANLPPSGLANAVAEGIRSEENIYTIEENVY
EVEEPNEYYCYVSSRQQPS.

The following sequences may not have activity in a CAR. In more embodiments, the TIM-3 human cytoplasmic domain does not consist of SEQ ID NO: 10 lacking amino acid residues 264-301 (see FIG. 10):

(SEQ ID NO: 12)
FKWYSHSKEKIQNLSLISLANLPPSGLANAVAEGIRSEEN.

In embodiments, the TIM-3 cytoplasmic domain does not consist of:

(SEQ ID NO: 35)
FKWYSHSKEKIQNLSLISLANLPPSGLANAVAEGIRSEENI.

Thus, in these embodiments, additional sequences of the cytoplasmic domain are required in addition to SEQ ID NO: 12 and SEQ ID NO: 35 for the CAR to be active.

In further embodiments, the TIM-3 human cytoplasmic domain comprises or consists of SEQ ID NO: 10, comprising an amino acid substitution at positions 265 (X₁) and/or 272 (X₂). In more embodiments, the TIM-3 human cytoplasmic domains comprises or consists of SEQ ID NO: 10, comprising an amino acid substitution at positions the tyrosines, including positions 265 (X₁) and/or 272 (X₂).

An exemplary TIM-3 human cytoplasmic domain comprises or consists of:

(SEQ ID NO: 13)
FKWYSHSKEKIQNLSLISLANLPPSGLANAVAEGIRSEENIX₁TIEENV
X₂EVEEPNEX₃X₄CX₅VSSRQQPSQPLGCRFAMP,

wherein X₁, X₂, X₃, X₄ and X₅ are tyrosine or alanine. (SEQ ID NO: 13).

In some embodiments, X₁ is an alanine. In further non-limiting examples, X₁ is an alanine and X₂ is a tyrosine. In other embodiments, X₂ is an alanine. In further non-limiting examples, X₁ is a tyrosine and X₂ is an alanine. In further embodiments, both X₁ and X₂ are alanine. In more embodiments, one or both of X₁ and X₂ are alanine, and X₃, X₄ and X₅ are tyrosine. In yet other embodiments, one or both of X₁ and X₂ are alanine, and one or more of X₃, X₄ and X₅ are alanine. In further embodiments, one or both of X₁ and X₂ are alanine, and one of X₃, X₄ and X₅ are alanine. In more embodiments, one or both of X₁ and X₂ are alanine, and two of X₃, X₄ and X₅ are alanine. In some embodiments, one or both of X₁ and X₂ are alanine, and all of X₃, X₄ and X₅ are alanine. In one specific non-limiting example, both of X₁ and X₂ are alanine, and all of X₃, X₄ and X₅ are alanine. In another specific non-limiting example, both of X₁ and X₂ are alanine, and none of X₃, X₄ and X₅ are alanine. In yet another non-limiting example, only one of X₁ and X₂ are alanine, and all of X₃, X₄ and X₅ are alanine. In a further specific non-limiting example, only one of X₁ and X₂ are alanine, and none of X₃, X₄ and X₅ are alanine.

In other embodiments, the TIM-3 human cytoplasmic domain comprises or consists of:

(SEQ ID NO: 13)
FKWYSHSKEKIQNLSLISLANLPPSGLANAVAEGIRSEENIX₁TIEENV
X₂EVEEPNEX₃X₄CX₅VSSRQQPSQPLGCRFAMP,

wherein X₁, X₂, X₃, X₄ and X₅ are tyrosine (Y), alanine (A), phenylalanine (F), aspartic acid (D), or glutamic acid (E) (SEQ ID NO: 36).

In some non-limiting examples, one, two, three, or four of X₁, X₂, X₃, X₄ and X₅ are tyrosine, and the remaining of X₁, X₂, X₃, X₄ and X₅ are alanine (A), phenylalanine (F), aspartic acid (D), or glutamic acid (E). In more non-limiting examples, X₁ and X₂ are alanine, and X₃, X₄ and X₅ are alanine (A), phenylalanine (F), aspartic acid (D), or glutamic acid (E). In further non-limiting examples, X₁ and X₂ are tyrosine (Y), and X₃, X₄ and X₅ are alanine (A), phenylalanine (F), aspartic acid (D), or glutamic acid (E).

In other embodiments, the TIM-3 human cytoplasmic domain comprises or consists of:

(SEQ ID NO: 37)
FKWYSHSKEKIQNLSLISLANLPPSGLANAVAEGIRSEENIX₁TIEENV
X₂EVEEPNEX₃X₄CX₅VSSRQQPS,

wherein X₁, X₂, X₃, X₄ and X₅ are tyrosine (Y), alanine (A), phenylalanine (F), aspartic acid (D), or glutamic acid (E).

In some non-limiting examples, one, two, three, or four of X₁, X₂, X₃, X₄ and X₅ are tyrosine, and the remaining of X₁, X₂, X₃, X₄ and X₅ are alanine (A), phenylalanine (F), aspartic acid (D), or glutamic acid (E). In more non-limiting examples, X₁ and X₂ are alanine, and X₃, X₄ and X₅ are alanine (A), phenylalanine (F), aspartic acid (D), or glutamic acid (E). In further non-limiting examples, X₁ and X₂ are tyrosine (Y), and X₃, X₄ and X₅ are alanine (A), phenylalanine (F), aspartic acid (D), or glutamic acid (E).

The present disclose is not limited to the use of human TIM-3 cytoplasmic domains. TIM-3 cytoplasmic domain from other species can function in the context of a CAR. These include, but are not limited to:

(SEQ ID NO: 38, mouse)
LKWYSYKKKKLSSLSLITLANLPPGGLANAGAVRIRSEENIYTIEENVY
EVENSNEYYCYVNSQQPS
(SEQ ID NO: 39, rat)
LKWYSSKKKKLQDLSLITLANSPPGGLVNAGAGRIRSEENIYTIEENIY
EMENSNEYYCYVSSQQPS.

In more embodiments, the TIM-3 cytoplasmic domain comprises:

(SEQ ID NO: 40)
SEENIYTIEENVYEVEE.

Without being bound by theory, this amino acid sequence imparts co-stimulatory activity.

The cytoplasmic domain can include only one intracellular signaling domain, or more than one intracellular signaling domains, such as, but not limited to, 2, 3 or 4 intracellular signaling domains responsible for activation of at least one of the normal effector functions of a T cell in which the CAR is expressed or placed in. Exemplary T cell signaling domains are provided herein, and are known to the person of ordinary skill in the art.

While an entire intracellular T cell signaling domain can be employed in a CAR, in many cases it is not necessary to use the entire chain. To the extent that a truncated portion of the intracellular T cell signaling domain is used, such truncated portion may be used in place of the intact chain as long as it transduces the relevant T cell effector function signal.

Examples of intracellular T cell signaling domains for use in the CAR include the cytoplasmic sequences of the T cell receptor (TCR) and co-stimulatory molecules that act in concert to initiate signal transduction following antigen receptor engagement, as well as any derivative or variant of these sequences and any synthetic sequence that has the same functional capability. T cell receptor signaling domains regulate primary activation of the T cell receptor complex either in a stimulatory way, or in an inhibitory way. The disclosed CARs can include primary cytoplasmic signaling sequences that act in a stimulatory manner, which may contain signaling motifs that are known as immunoreceptor tyrosine-based activation motifs or ITAMs. Examples of ITAM containing primary cytoplasmic signaling sequences that can be included in a disclosed CAR include those from CD3 zeta, FcR gamma, FcR beta, CD3 gamma, CD3 delta, CD3 epsilon, CD22, CD79a, CD79b, and CD66d proteins. In several embodiments, the cytoplasmic signaling molecule in the CAR includes an intracellular T cell signaling domain from CD3 zeta. In some embodiments, the CAR can include 1, 2, 3, or 4 intracellular signaling domains, such as a 4-1BB, CD3 zeta, FcR gamma, FcR beta, CD3 gamma, CD3 delta, CD3 epsilon CD22, CD28, CD79a, or CD79b intracellular signaling domain.

The intracellular region of the CAR can include the ITAM containing primary cytoplasmic signaling domain (such as CD3-zeta) by itself or combined with any other desired cytoplasmic domain(s) useful in the context of a CAR. For example, the cytoplasmic domain of the CAR can include a CD3 zeta chain portion and an additional intracellular costimulatory signaling domain. The costimulatory signaling domain refers to a portion of the CAR including the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX₄₀ (CD134), CD30, CD40, PD-1, ICOS, lymphocyte function-associated antigen 1 (LFA-1), CD2, CD7, LIGHT, NKG2C, and B7-H3. An additional example of a signaling domain that can be included in a disclosed CARs is a Tumor necrosis factor receptor superfamily member 18 (TNFRSF18; also known as glucocorticoid-induced TNFR-related protein, GITR) signaling domain.

The chimeric antigen receptor can include an additional intracellular co-stimulatory signaling domain a) C-terminal to the TIM-3 cytoplasmic domain or b) between the TIM-3 cytoplasmic domain and the intracellular signaling domain. In some embodiments, wherein the intracellular co-stimulatory signaling domain is a CD27, CD28, 4-1BB (CD137), OX₄₀ (CD134), CD30, CD40, PD-1, ICOS, lymphocyte function-associated antigen 1 (LFA-1), CD2, CD7, or B7-H3 intracellular co-stimulatory signaling domain. In a specific non-limiting example, the intracellular co-stimulatory signaling domain is a 4-1BB intracellular signaling domain.

In some embodiments, the CAR can include a TIM-3 cytoplasmic domain and one or more of a CD3 zeta signaling domain, a CD8 signaling domain, a CD28 signaling domain, a 4-1BB signally domain. In other embodiments, the CAR can include a TIM-3 cytoplasmic domain and two or more of a CD3 zeta signaling domain, a CD8 signaling domain, a CD28 signaling domain, a 4-1BB signally domain. In one embodiment, the cytoplasmic domain includes the signaling domain of CD3-zeta and the signaling domain of CD28. In another embodiment, the cytoplasmic domain includes the signaling domain of CD3 zeta and the signaling domain of 4-1BB. In yet another embodiment, the cytoplasmic domain includes the signaling domain of CD3-zeta and the signaling domain of CD28 and 4-1BB. The order of the one or more T cell signaling domains on the CAR can be varied as needed by the person of ordinary skill in the art.

Exemplary amino acid sequences for such T cell signaling domains are provided. For example, the CD3 zeta signaling domain can include or consist of the amino acid sequence set forth as RVKFSRSADAPAYQQGQNQLYNELNLGRREEYDVLDKRRGRDPEMGGKPRRKNPQEGLY NELQKDKMAEAYSEIGMKGERRRGKGHDGLYQGLSTATKDTYDALHMQALPPR (SEQ ID NO: 14), the CD8 signaling domain can include or consist of the amino acid sequence set forth as SEQ ID NO: 15 (FVPVFLPAKPTTTPAPRPPTPAPTIASQPLSLRPEACRPAAGGAVHTRGLDFACDIYIWAPL AGTCGVLLLSLVITLYCNHRNR (SEQ ID NO: 15), the CD28 signaling domain can include or consist of the amino acid sequence set forth as SKRSRLLHSDYMNMTPRRPGPTRKHYQPYAPPRDFAAYRS (SEQ ID NO: 16), the CD137 signaling domain can include or consist of the amino acid sequences set forth as KRGRKKLLYIFKQPFMRPVQTTQEEDGCSCRFPEEEEGGCEL (SEQ ID NO: 17) or RFSVVKRGRKKLLYIFKQPFMRPVQTTQEEDGCSCRFPEEEEGGCEL(SEQ ID NO: 18).

The cytoplasmic signaling sequences within the cytoplasmic signaling portion of the CAR of the invention may be linked to each other in a random or specified order. An additional intracellular co-stimulator domain can be included C-terminal to the TIM-3 cytoplasmic domain, or between the TIM-3 cytoplasmic domain and the intracellular signaling domain, such as, but not limited to CD3-zeta. This intracellular co-stimulator signaling domain can be CD27, CD28, 4-BB, OX₄₀, CD30, CD40, DP-1, ICOS, LFA-1, CD2, CD7 or a B7H3 intracellular co-stimulatory signaling domain. In a specific non-limiting example, the intracellular co-stimulatory signaling domain is CD28. In another specific non-limiting example, intracellular co-stimulatory signaling domain is 4-1BB. Optionally, a short polypeptide linker, preferably between 2 and 10 amino acids in length may form the linkage. A glycine-serine doublet provides a particularly suitable linker. Further, between the signaling domain and the transmembrane domain of the CAR, there may be a spacer domain, which includes a polypeptide sequence. The spacer domain may comprise up to 300 amino acids, preferably 10 to 100 amino acids and most preferably 25 to 50 amino acids.

### Additional Description of CARs

Also provided are functional portions of the CARs described herein. The term “functional portion” when used in reference to a CAR refers to any part or fragment of the CAR, which part or fragment retains the biological activity of the CAR of which it is a part (the parent CAR). Functional portions encompass, for example, those parts of a CAR that retain the ability to recognize target cells, or detect, treat, or prevent a disease, to a similar extent, the same extent, or to a higher extent, as the parent CAR. In reference to the parent CAR, the functional portion can comprise, for instance, about 10%, 25%, 30%, 50%, 68%, 80%, 90%, 95%, or more, of the parent CAR. The CAR can be a first generation, second generation, third generation, or forth generation CAR.

The CAR or functional portion thereof, can include additional amino acids at the amino or carboxy terminus, or at both termini, which additional amino acids are not found in the amino acid sequence of the parent CAR. Desirably, the additional amino acids do not interfere with the biological function of the CAR or functional portion, e.g., recognize target cells, detect cancer, treat or prevent cancer, etc. More desirably, the additional amino acids enhance the biological activity, as compared to the biological activity of the parent CAR.

Also provided are functional variants of the CARs described herein, which have substantial or significant sequence identity or similarity to a parent CAR, which functional variant retains the biological activity of the CAR of which it is a variant. Functional variants encompass, for example, those variants of the CAR described herein (the parent CAR) that retain the ability to recognize target cells to a similar extent, the same extent, or to a higher extent, as the parent CAR. In reference to the parent CAR, the functional variant can, for instance, be at least about 30%, about 50%, about 75%, about 80%, about 85%, about 90%, about 91%, about 92%, about 93%, about 94%, about 95%, about 96%), about 97%, about 98%, about 99% or more identical in amino acid sequence to the parent CAR.

A functional variant can, for example, comprise the amino acid sequence of the parent CAR with at least one conservative amino acid substitution. The CAR can include at most 10, 9, 8, 7, 6, 5, 4, 3 2 or 1 conservative amino acid substitutions. Alternatively, or additionally, the functional variants can comprise the amino acid sequence of the parent CAR with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR.

The CARs (including functional portions and functional variants) can be of any length, i.e., can comprise any number of amino acids, provided that the CARs (or functional portions or functional variants thereof) retain their biological activity, e.g., the ability to specifically bind to antigen, detect diseased cells in a mammal, or treat or prevent disease in a mammal, etc. For example, the CAR can be about 50 to about 5000 amino acids long, such as 50, 70, 75, 100, 125, 150, 175, 200, 300, 400, 500, 600, 700, 800, 900, 1000 or more amino acids in length.

The CARs (including functional portions and functional variants of the invention) can comprise synthetic amino acids in place of one or more naturally occurring amino acids. Such synthetic amino acids are known in the art, and include, for example, aminocyclohexane carboxylic acid, norleucine, a-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1 ,2,3,4- tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, oc- aminocycloheptane carboxylic acid, -(2-amino-2-norbornane)-carboxylic acid, γ-diaminobutyric acid, α,β-diaminopropionic acid, homophenylalanine, and α-tert-butylglycine.

The CARs (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized, or conjugated.

Methods of generating chimeric antigen receptors, T cells including such receptors, and their use (e.g., for treatment of cancer) are known in the art and further described herein (see, e.g., Brentjens et al., 2010, Molecular Therapy, 18: 4, 666-668; Morgan et al., 2010, Molecular Therapy, published online Feb. 23, 2010, pages 1 -9; Till et al., 2008, Blood, 1 12 :2261 -2271; Park et al., Trends Biotechnol., 29: 550-557, 2011; Grupp et al., N Engl J Med., 368: 1509-1518, 2013; Han et al., J. Hematol Oncol., 6: 47, 2013; PCT Pub. WO2012/079000, WO2013/126726; and U.S. Pub. 2012/0213783, each of which is incorporated by reference herein in its entirety.) For example, a nucleic acid molecule encoding a disclosed chimeric antigen binding receptor can be included in an expression vector (such as a lentiviral vector) for expression in a host cell, such as a T cell, to make the disclosed CAR. In some embodiments, methods of using the chimeric antigen receptor include isolating T cells from a subject, transforming the T cells with an expression vector (such as a lentiviral vector) encoding the chimeric antigen receptor, and administering the engineered T cells expressing the chimeric antigen receptor to the subject for treatment, for example for treatment of a tumor in the subject.

### Nucleic Acid Molecules and Host Cells

Nucleic acid sequences encoding the CARs that specifically bind an antigen of interest, such as, but not limited to, tumor antigens such as CD22, CD123, CEA, EGFRVIII, ErbB2, HER2, IL-13ralpha2, MUCI, CD19 or CD20, can be prepared by any suitable method including, for example, cloning of appropriate sequences or by direct chemical synthesis by methods such as the phosphotriester method of Narang et al., Meth. Enzymol. 68: 90-99, 1979; the phosphodiester method of Brown et al., Meth. Enzymol. 68: 109-151, 1979; the diethylphosphoramidite method of Beaucage et al., Tetra. Lett. 22: 1859-1862, 1981; the solid phase phosphoramidite triester method described by Beaucage & Caruthers, Tetra. Letts. 22(20): 1859-1862, 1981, for example, using an automated synthesizer as described in, for example, Needham-VanDevanter et al., Nucl. Acids Res. 12: 6159-6168, 1984; and, the solid support method of U.S. Pat. No. 4,458,066. Chemical synthesis produces a single stranded oligonucleotide. This can be converted into double stranded DNA by hybridization with a complementary sequence or by polymerization with a DNA polymerase using the single strand as a template.

Exemplary nucleic acids can be prepared by cloning techniques. Examples of appropriate cloning and sequencing techniques, and instructions sufficient to direct persons of skill through many cloning exercises are known (see, e.g, Sambrook et al. (Molecular Cloning: A Laboratory Manual, 4^{th} ed, Cold Spring Harbor, New York, 2012) and Ausubel et al. (In Current Protocols in Molecular Biology, John Wiley & Sons, New York, through supplement 104, 2013). Product information from manufacturers of biological reagents and experimental equipment also provide useful information. Such manufacturers include the SIGMA Chemical Company (Saint Louis, MO), R&D Systems (Minneapolis, MN), Pharmacia Amersham (Piscataway, NJ), CLONTECH Laboratories, Inc. (Palo Alto, CA), Chem Genes Corp., Aldrich Chemical Company (Milwaukee, WI), Glen Research, Inc., GIBCO BRL Life Technologies, Inc. (Gaithersburg, MD), Fluka Chemica-Biochemika Analytika (Fluka Chemie AG, Buchs, Switzerland), Invitrogen (Carlsbad, CA), and Applied Biosystems (Foster City, CA), as well as many other commercial sources known to one of skill. Nucleic acids molecules can be codon-optimized, such as for expression in human cells.

Nucleic acids can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill. Modifications can be made to a nucleic acid encoding a polypeptide described herein without diminishing its biological activity. Some modifications can be made to facilitate the cloning, expression, or incorporation of the targeting molecule into a fusion protein. Such modifications are well known to those of skill in the art and include, for example, termination codons, a methionine added at the amino terminus to provide an initiation, site, additional amino acids placed on either terminus to create conveniently located restriction sites, or additional amino acids (such as poly His) to aid in purification steps. In addition to recombinant methods, the CARs of the present disclosure can also be constructed in whole or in part using standard peptide synthesis well known in the art.

The nucleic acid molecule encoding the CAR can operably linked to a promoter. The nucleic acid molecule encoding the CAR can be included in a vector (such as a lentiviral vector or gamma retroviral vector) for expression in a host cell. Exemplary cells are mammalian cells, and include a T cell, such as a cytotoxic T lymphocyte (CTL) or a regulatory T cell, and a NK cell. In specific non-limiting examples, the cell is a T cell, such as a CD3⁺ T cell. The CD3⁺ T cell can be a CD4⁺ or a CD8⁺ T cell. In other specific non-limiting examples, the cell is a NK cell. Methods of generating nucleic acid molecules encoding chimeric antigen receptors and T cells (or NK cells) including such receptors are known in the art (see, e.g., Brentjens et al., 2010, Molecular Therapy, 18: 4, 666-668; Morgan et al., 2010, Molecular Therapy, published online February 23, 2010, pages 1 -9; Till et al., 2008, Blood, 1 12: 2261 -2271; Park et al., Trends Biotechnol., 29: 550-557, 2011; Grupp et al., N Engl J Med., 368: 1509-1518, 2013; Han et al., J. Hematol Oncol., 6: 47, 2013; PCT Publication Nos. WO2012/079000, WO2013/126726; and U.S. Published Application No. 2012/0213783, each of which is incorporated by reference herein in its entirety).

If of interest, once expressed, a CAR can be purified according to standard procedures in the art, including ammonium sulfate precipitation, affinity columns, column chromatography, and the like (see, generally, Simpson ed., Basic methods in Protein Purification and Analysis: A laboratory Manual, Cold Harbor Press, 2008). These molecules need not be 100% pure. Once purified, partially or to homogeneity as desired, if to be used therapeutically, the polypeptides should be substantially free of endotoxin. Additional methods for expression and purification are known in the art, see, e.g., Harlow and Lane, Antibodies: A Laboratory Manual, 2^{nd}, Cold Spring Harbor Laboratory, New York, 2013, Simpson ed., Basic methods in Protein Purification and Analysis: A laboratory Manual, Cold Harbor Press, 2008, and Ward et al., Nature 341: 544, 1989.

The nucleic acid molecules also can be expressed in a recombinantly engineered cell such as bacteria, plant, yeast, insect and mammalian cells. The CAR can be expressed as a fusion protein. Methods of expressing and purifying proteins, including CARs, antibodies and antigen binding molecules, are known and further described herein (see, e.g., Al-Rubeai (ed), Antibody Expression and Production, Springer Press, 2011). Those of skill in the art are knowledgeable in the numerous expression systems available for expression of proteins including E. coli, other bacterial hosts, yeast, and various higher eukaryotic cells such as the COS, CHO, HeLa and myeloma cell lines. The term “host cell” also includes any progeny of the subject host cell. It is understood that all progeny may not be identical to the parental cell since there may be mutations that occur during replication. Methods of stable transfer, meaning that the foreign DNA is continuously maintained in the host, are known in the art. As disclosed herein, specific embodiments of the present disclosure include T cells, such as human T cells and human NK cells, which express the CAR. These T cells can be CD3⁺ T cells, such as CD4⁺ or CD8⁺ T cells. If of interest, once expressed, a CAR can be purified according to standard procedures in the art, including ammonium sulfate precipitation, affinity columns, column chromatography, and the like (see, generally, Simpson ed., Basic methods in Protein Purification and Analysis: A laboratory Manual, Cold Harbor Press, 2008). The CARs need not be 100% pure. Once purified, partially or to homogeneity as desired, if to be used therapeutically, the polypeptides should be substantially free of endotoxin. Additional methods for expression and purification are known in the art, see, e.g., Harlow and Lane, Antibodies: A Laboratory Manual, 2^{nd}, Cold Spring Harbor Laboratory, New York, 2013, Simpson ed., Basic methods in Protein Purification and Analysis: A laboratory Manual, Cold Harbor Press, 2008, and Ward et al., Nature 341: 544, 1989.

The expression of nucleic acids encoding the CARs described herein can be achieved by operably linking the DNA encoding the CAR to a promoter (which is either constitutive or inducible), followed by incorporation into an expression cassette. The promoter can be any promoter of interest, including a cytomegalovirus promoter and a human T cell lymphotrophic virus promoter (HTLV)-1. Optionally, an enhancer, such as a cytomegalovirus enhancer, is included in the construct. The cassettes can be suitable for replication and integration in either prokaryotes or eukaryotes. Typical expression cassettes contain specific sequences useful for regulation of the expression of the DNA encoding the protein. For example, the expression cassettes can include appropriate promoters, enhancers, transcription and translation terminators, initiation sequences, a start codon (i.e., ATG) in front of a protein-encoding gene, splicing signal for introns, sequences for the maintenance of the correct reading frame of that gene to permit proper translation of mRNA, and stop codons. The vector can encode a selectable marker, such as a marker encoding drug resistance (for example, ampicillin or tetracycline resistance).

To obtain high level expression of a cloned gene, it is desirable to construct expression cassettes which contain, at the minimum, a strong promoter to direct transcription, a ribosome binding site for translational initiation (internal ribosomal binding sequences), and a transcription/translation terminator. For E. coli, this can include a promoter such as the T7, trp, lac, or lambda promoters, a ribosome binding site, and preferably a transcription termination signal. For eukaryotic cells, the control sequences can include a promoter and/or an enhancer derived from, for example, an immunoglobulin gene, HTLV, SV40 or cytomegalovirus, and a polyadenylation sequence, and can further include splice donor and/or acceptor sequences (for example, CMV and/or HTLV splice acceptor and donor sequences). The cassettes can be transferred into the chosen host cell by well-known methods such as transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

When the host is a eukaryote, such methods of transfection of DNA as calcium phosphate coprecipitates, conventional mechanical procedures such as microinjection, electroporation, insertion of a plasmid encased in liposomes, or virus vectors may be used. Eukaryotic cells can also be co-transformed with polynucleotide sequences encoding the CAR, and a second foreign DNA molecule encoding a selectable phenotype, such as the herpes simplex thymidine kinase gene. Another method is to use a eukaryotic viral vector, such as simian virus 40 (SV40), a lentivirus or bovine papilloma virus, to transiently infect or transform eukaryotic cells and express the protein (see for example, Viral Expression Vectors, Springer press, Muzyczka ed., 2011). One of skill in the art can readily use an expression system such as plasmids and vectors of use in producing proteins in cells including higher eukaryotic cells such as the COS, CHO, HeLa and myeloma cell lines.

In some embodiments, a viral vector is utilized for expression of the CAR. Viral vectors include, but are not limited to simian virus 40, adenoviruses, adeno-associated virus (AAV), lentiviral vectors, and retroviruses, such as gamma retroviruses. Retroviral vectors provide a highly efficient method for gene transfer into eukaryotic cells. Moreover, retroviral integration takes place in a controlled fashion and results in the stable integration of one or a few copies of the new genetic information per cell. Without being bound by theory, lentiviral vectors have the advantage over vectors derived from onco-retroviruses such as murine leukemia viruses in that they can transduce non-proliferating cells, such as hepatocytes. They also have the added advantage of low immunogenicity. The use of lentiviral vectors to express a CAR is known in the art, and is disclosed for example in U.S. Application No. 2014/0050708, which is incorporated herein by reference.

In some embodiments, host cells are produced for introduction into s subject of interest. The host cell can be a peripheral blood lymphocyte (PBL) or a peripheral blood mononuclear cell (PBMC), a purified T cell, or a purified NK cell. The T cell can be any T cell, such as a cultured T cell, e.g., a primary T cell, or a T cell from a cultured T cell line, e.g., Jurkat, SupTI, etc., or a T cell obtained from a mammal (such as a human patient to which the CAR-T cell will later be administered). If obtained from a mammalian subject, such as a human subject, the T cell can be obtained from numerous sources, including but not limited to blood, bone marrow, lymph node, the thymus, or other tissues or fluids. T cells can also be enriched for or purified. The T cell can be any type of T cell and can be of any developmental stage, including but not limited to, CD3⁺ cells, CD4⁺/CD8⁺ double positive T cells, CD4⁺ helper T cells, e.g., Th₁ and Th₂ cells, CD8⁺ T cells (e.g., cytotoxic T cells), tumor infiltrating cells, memory T cells, naive T cells, and the like. The T cell may be a CD3⁺ T cell, such as a CD8⁺ T cell or a CD4⁺ T cell. In alternative embodiments, the cell can be an NK cells, such as an NK cell obtained from the same subject to which the CAR-NK cell will later be administered.

Also provided is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any recombinant expression vector, or a cell other than a T cell, e.g., a B cell, a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly host cells (e.g., consisting essentially of) comprising the recombinant expression vector encoding the CAR. The population also can be a clonal population of cells, in which all cells of the population are clones of a single host cell comprising a recombinant expression vector, such that all cells of the population comprise the recombinant expression vector. In one embodiment of the invention, the population of cells is a clonal population comprising host cells comprising a recombinant expression vector as described herein. The T cells can be CD3+T cells, such as CD8⁺ T cell or a CD4⁺ T cells. In some embodiments, the T cells are transformed with Epstein Barr virus, see Savoldo et al., Blood 110: 2620-2630, 2007, incorporated herein by reference. In other embodiments, the cells are heterologous to a recipient (see below), and are deleted for a HLA class I and/or T cell receptor, so they do not provoke a graft versus host disease (GVHD) or host versus graft reaction. The cells can also be NK cells. The cells can be autologous to a recipient or allogeneic. These populations are of use in any of the methods disclosed herein.

### Methods of Treatment and Pharmaceutical Compositions

Disclosed herein are methods for treating a tumor, wherein the cells of the tumor express a tumor antigen. In some embodiments, the tumor is a malignancy, such as a lymphoid malignancy. In other embodiments, the tumor can be a solid tumor. In several non-limiting examples, the tumor is breast, ovarian, gastric, esophageal, skin, lung or head and neck cancer. The method an include selecting a subject with the tumor for treatment, and/or diagnosing expression of a specific tumor antigen by the cells of the tumor. The subject can be a human or veterinary subject. In some embodiments, the CAR includes an scFv that specifically binds CD19 or CD20, and the subject has a B cell malignancy, such as, but not limited to, diffuse large B cell lymphoma (DLBCL), B cell acute lymphoblastic leukemias (B-ALL), chronic lymphocytic leukemia (CLL), or non-Hodgkin lymphoma.

The method includes administering to the subject a therapeutically effective amount of the pharmaceutical composition including a vector, such as a lentiviral vector encoding a CAR, and/or administering a therapeutically effective amount of a pharmaceutical composition comprising cells, such as T cells and/or NK cells, that express the CAR, wherein the antigen binding portion of the CAR specifically binds an tumor antigen. In some embodiments, the method includes selecting a subject that expresses a tumor antigen, such as, but not limited to CD22, CD123, CEA, EGFRVIII, ErbB2, HER2, IL-13ralpha2, MUC1, CD19 or CD20, and administering a CAR that targets the respective tumor antigen.

Pharmaceutical compositions can include a CAR-expressing cell, e.g., a plurality of CAR-expressing cells, as described herein, in combination with one or more pharmaceutically or physiologically acceptable carriers, diluents or excipients. The CAR-expressing cells can be T cells, such as CD3⁺ T cells, such as CD4⁺ and/or CD8⁺ T cells, and/or NK cells. Such compositions may include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

The cells can be autologous to the recipient. However, the cells can also be heterologous (allogeneic). In some embodiments, the cells are T cells, such as T cells transformed with Epstein Barr virus, see Savoldo et al., Blood 110: 2620-2630, 2007, incorporated herein by reference. In other embodiments, the cells are heterologous (allogeneic) to a recipient (see below), and are deleted for an HLA class I and/or T cell receptor, so they do not provoke a graft versus host disease (GVHD) or host versus graft reaction.

With regard to the cells, a variety of aqueous carriers can be used, for example, buffered saline and the like, for introducing the cells. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration in these formulations can vary widely, and will be selected primarily based on fluid volumes, viscosities, body weight and the like in accordance with the particular mode of administration selected and the subject's needs.

In one embodiment, the pharmaceutical composition is substantially free of, e.g., there are no detectable levels of a contaminant, such as endotoxin, mycoplasma, replication competent lentivirus (RCL), p24, residual anti-CD3/anti-CD28 coated beads, mouse antibodies, pooled human serum, bovine serum albumin, bovine serum, culture media components, vector packaging cell or plasmid components, a bacterium and a fungus.

The precise amount of the composition to be administered can be determined by a physician with consideration of individual differences in age, weight, tumor size, extent of metastasis, and condition of the patient (subject). It can generally be stated that a pharmaceutical composition comprising the T cells (and/or NK cells) described herein may be administered at a dosage of 10⁴ to 10⁹ cells/kg body weight, such as 10⁵ to 10⁶ cells/kg body weight, including all integer values within those ranges. Exemplary doses are 10⁶ cells/kg to about 1×10⁸ cells/kg, such as from about 5×10⁶ cells/kg to about 7.5×10⁷ cells/kg, such as at about 2.5×10⁷ cells/kg, or at about 5.0×10⁷ cells/kg.

A composition can be administered once or multiple times, such as 2, 3,4, 5, 6, 7, 8, 9, or 10 times at these dosages. The composition can be administered by using infusion techniques that are commonly known in immunotherapy (see, e.g., Rosenberg et al., New Eng. J. of Med. 319: 1676, 1988). The compositions can be administered daily, weekly, bimonthly or monthly. In some non-limiting examples, the composition is formulated for intravenous administration and is administered multiple times. The quantity and frequency of administration will be determined by such factors as the condition of the subject, and the type and severity of the subject's disease, although appropriate dosages may be determined by clinical trials.

In one embodiment, the CAR is introduced into cells, such T cells or NK cells, and the subject receives an initial administration of cells, and one or more subsequent administrations of the cells, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the CAR cells is administered to the subject (e.g., human) per week, e.g., 2, 3, or 4 administrations of the CAR cells of the invention are administered per week. In one embodiment, the subject receives more than one administration of the CAR T cells per week (e.g., 2, 3 or 4 administrations per week) (also referred to as a cycle), followed by a week of no CAR cells administrations, and then one or more additional administration of the CAR cells (e.g., more than one administration of the CAR T cells per week) is administered to the subject. In another embodiment, the subject (e.g., human subject) receives more than one cycle of CAR cells, and the time between each cycle is less than 10, 9, 8, 7, 6, 5, 4, or 3 days. In one embodiment, the CAR cells are administered every other day for 3 administrations per week. In another embodiment, the CAR cells are administered for at least two, three, four, five, six, seven, eight or more weeks. The dosage of the above treatments to be administered to a patient will vary with the precise nature of the condition being treated and the recipient of the treatment. The scaling of dosages for human administration can be performed according to art-accepted practices.

In some embodiments, CAR-modified T cells are able to replicate in vivo resulting in long-term persistence that can lead to sustained tumor control. In various aspects, the T cells administered to the subject, or the progeny of these cells, persist in the subject for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, or for years after administration of the T cell to the subject. In other embodiments, the cells and their progeny are present for less than six months, five month, four months, three months two months, or one month, e.g., three weeks, two weeks, one week, after administration of the T cell to the subject.

The administration of the subject compositions may be carried out in any convenient manner, including by injection, ingestion, transfusion, implantation or transplantation. The disclosed compositions can be administered to a patient trans arterially, subcutaneously, intradermally, intratumorally, intranodally, intramedullary, intramuscularly, by intravenous (i.v.) injection, or intraperitoneally. In some embodiments, the compositions are administered to a patient by intradermal or subcutaneous injection. In other embodiments, the compositions of the present invention are administered by i.v. injection. The compositions can also be injected directly into a tumor or lymph node.

In some embodiments, subjects can undergo leukapheresis, wherein leukocytes are collected, enriched, or depleted ex vivo to select and/or isolate the cells of interest, e.g., T cells and or NK cells. These cell isolates may be expanded by methods known in the art and treated such that one or more CAR constructs can be introduced, thereby creating an autologous cell that express the CAR. In one aspect, CAR expressing cells are generated using lentiviral viral vectors.

In some embodiments, the T and/or NK cells are autologous. In other embodiments, the T cells and/or NK cells are allogeneic. The T calls and/or NK cells are then introduced into the subject, as disclosed above. In one embodiment, the cells transiently express the vector for 4, 5, 6, 7, 8, 9, 10, 11, 12, 13, 14, 15 days after transduction. In one non-limiting example, the vector is transduced into the T cell by electroporation.

In some embodiments, a subject is administered a therapeutically effective amount of T cells and/or NK cells expressing the disclosed CAR. In particular embodiments (see U.S. Published Application No. US20140271635 A1, incorporated herein by reference), prior to expansion and genetic modification, a source of T cells is obtained from a subject.

The term “subject” is intended to include living organisms in which an immune response can be elicited (e.g., mammals). Examples of subjects include humans, dogs, cats, mice, rats, pigs (and other veterinary subjects) and non-human primates. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In other embodiments, any number of T cell lines available in the art, may be used. In some non-limiting examples, T cells and/or NK cells can be obtained from a unit of blood collected from a subject using any number of techniques known to the skilled artisan, such as FICOLL™ separation, or the cells can be obtained by apheresis. The apheresis product typically contains lymphocytes, including T cells, monocytes, granulocytes, B cells, NK cells, other nucleated white blood cells, red blood cells, and platelets. In some specific non-limiting examples, the cells are autologous.

Cells collected by apheresis can be washed to remove the plasma fraction and to place the cells in an appropriate buffer or media for subsequent processing steps. In some non-limiting examples, the cells are washed with phosphate buffered saline (PBS). In alternative examples, the wash solution lacks calcium and may lack magnesium or may lack many if not all divalent cations. Initial activation steps in the absence of calcium can lead to magnified activation. The washing step can be accomplished by methods known in the art, such as by using a semi-automated “flow-through” centrifuge (for example, the Cobe 2991 cell processor, the Baxter CYTOMATE®, or the HAEMONETICS CELL SAVER 5®) according to the manufacturer's instructions. After washing, the cells can be resuspended in a variety of biocompatible buffers, such as a saline solution with or without buffer. Alternatively, the undesirable components of the apheresis sample can be removed and the cells directly resuspended in culture media.

In some embodiments, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+ T cells, can be further isolated by positive or negative selection techniques. For example, T cells can be isolated by incubation with anti-CD3/anti-CD28 (e.g., 3×28)-conjugated beads, such as DYNABEADS® M-450 CD3/CD28 T, for a time period sufficient for positive selection of the desired T cells, see U.S. Published Application No. US20140271635 A1. In a non-limiting example, the time period is about 30 minutes. In other non-limiting examples, the time period ranges from 30 minutes to 36 hours or longer and all integer values there between. In further non-limiting examples, the time period is at least 1, 2, 3, 4, 5, 6 hours, 10 to 24 hours, 24 hours or longer. Longer incubation times can be used to isolate T cells in any situation where there are few T cells as compared to other cell types, such in isolation from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. Multiple rounds of selection can also be used.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. A T cell population can be selected that expresses one or more cytokines. Methods for screening for cell expression are disclosed in PCT Publication No. WO 2013/126712.

For isolation of a desired population of cells by positive or negative selection, the concentration of cells and surface (e.g., particles such as beads) can be varied. Into ensure maximum contact of cells and beads. In some embodiments, a concentration of 1 billion cells/ml is used. In further embodiments, greater than 100 million cells/ml is used. In other embodiments, a concentration of cells of 10, 15, 20, 25, 30, 35, 40, 45, 50, 65, 70, 75, 80, 85, 90, 95, or 100 million cells/ml is used. Without being bound by theory, using high concentrations can result in increased cell yield, cell activation, and cell expansion. Lower concentrations of cells can also be used. Without being bound by theory, significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In some embodiments, the concentration of cells used is 5×10⁶/ml. In other embodiments, the concentration used can be from about 1×10⁵/ml to 1×10⁶/ml, and any integer value in between.

Cells can be incubated on a rotator for varying lengths of time at varying speeds at either 2-10° C., or at room temperature. T cells for stimulation can also be frozen after a washing step. Without being bound by theory, the freeze and subsequent thaw step provides a more uniform product by removing granulocytes and to some extent monocytes in the cell population. After the washing step that removes plasma and platelets, the cells can be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and Plasmalyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen, see U.S. Publication No. US-2014-0271635 A1.

Blood samples or apheresis product can be collected from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one aspect a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the T cells may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH®, anti-CD3 antibodies, cytoxan, fludarabine, cyclosporin, FK506, rapamycin, mycophenolic acid, steroids, FR901228, and irradiation. In certain aspects, cryopreserved cells are thawed and washed as described herein and allowed to rest for one hour at room temperature prior to use. Blood samples or apheresis product can be collected from a subject when needed, and not frozen.

T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

T cells can be expanded by contact with a surface having attached thereto an agent that stimulates a CD3/TCR complex associated signal and a ligand that stimulates a costimulatory molecule on the surface of the T cells. In some non-limiting examples, T cell populations may be stimulated, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8): 3975-3977, 1998; Haanen et al., J. Exp. Med. 190(9): 13191328, 1999; Garland et al., J. Immunol. Meth. 227(1-2): 53-63, 1999).

Once a CAR is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Isolated immune cells expressing a CAR, such as T cells, for example CD3⁺ T cells such as CD4⁺ and/or CD8⁺ T cells, and/or NK cells, can be administered in a pharmaceutically acceptable carrier, such as buffered saline or another medium suitable for administration to a subject. The cells can be administered in conjunction with other cells, or in the absence of other cells. In one embodiment, compositions containing isolated populations of cells can also contain one or more additional pharmaceutical agents, such as one or more anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine), depleting agents (for example, fludarabine, etoposide, doxorubicin, or vincristine), or non-steroidal anti-inflammatory agents such as acetylsalicylic acid, ibuprofen or naproxen sodium), cytokines (for example, interleukin-2), or a vaccine. Many chemotherapeutic agents are presently known in the art. In one embodiment, the chemotherapeutic agent is selected from the group consisting of mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones, e.g. anti-androgens, and anti-angiogenesis agents.

In other embodiments, a subject is administered the DNA encoding the CAR, to provide in vivo production. Immunization by nucleic acid constructs is well known in the art and taught, for example, in U.S. Pat. Nos. 5,643,578, and 5,593,972 and 5,817,637. U.S. Patent No. 5,880,103 describes several methods of delivery of nucleic acids encoding to an organism. The methods include liposomal delivery of the nucleic acids.

One approach to administration of nucleic acids is direct administration with plasmid DNA, such as with a mammalian expression plasmid. The nucleotide sequence encoding the disclosed CARs, can be placed under the control of a promoter to increase expression.

In another approach to using nucleic acids, a disclosed CAR can also be expressed by attenuated viral hosts or vectors or bacterial vectors. Recombinant vaccinia virus, adeno-associated virus (AAV), herpes virus, retrovirus, cytomegalovirus or other viral vectors can be used to express the antibody. For example, vaccinia vectors and methods useful protocols are described in U.S. Pat. No. 4,722,848. BCG (Bacillus Calmette Guerin) provides another vector for expression of the disclosed antibodies (see Stover, Nature 351: 456-460, 1991). In a specific non-limiting example, the vector is a lentiviral vector.

In one embodiment, a nucleic acid encoding a disclosed CAR, is introduced directly into cells. For example, the nucleic acid can be loaded onto gold microspheres by standard methods and introduced by a device such as Bio-Rad's HELIOS™ Gene Gun. The nucleic acids can be “naked,” consisting of plasmids under control of a strong promoter. The nucleic acid can be RNA; RNA encoding the CAR can be directly administered to the cells. In some embodiments, the cells are NK cells or T cells.

For the treatment of a tumor, the method can also include administering to the subject a therapeutically effective amount of an additional chemotherapeutic agent, surgery or radiation. In some embodiments, the malignancy is a lymphoid malignancy. The subject can also have a solid tumor, such as, but not limited to, breast cancer, ovarian cancer, gastric cancer or esophageal cancer. In other embodiments, the lymphoid malignancy is a diffuse large B cell lymphoma (DLBCL), a B cell acute lymphoblastic leukemia (B-ALL), a chronic lymphocytic leukemia (CLL), or non-Hodgkin lymphoma. Subjects can be selected for treatment that have these tumors.

The chemotherapeutic agent can be an antibody. Antibodies may be provided in lyophilized form and rehydrated with sterile water before administration, although they are also provided in sterile solutions of known concentration. The antibody solution is then added to an infusion bag containing 0.9% sodium chloride, USP, and typically administered at a dosage of from 0.5 to 15 mg/kg of body weight. Considerable experience is available in the art in the administration of antibody drugs, which have been marketed in the U.S. since the approval of RITUXAN® in 1997. The antibody can specifically bind programmed death (PD)-1 or programmed death ligand (PD-L1) (see below). Antibodies can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30 minute period if the previous dose was well tolerated.

In further embodiments for the treatment of malignancies, a CAR-expressing cell described herein may be used in a treatment regimen in combination with surgery, chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, cyclosporin, FK506, rapamycin, mycophenolic acid, steroids, FR901228, cytokines, and irradiation. peptide vaccine, such as that described in Izumoto et al. 2008 J Neurosurg 108: 963-971. Exemplary chemotherapeutic agents include an anthracycline (e.g., doxorubicin (e.g., liposomal doxorubicin)). a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

General Chemotherapeutic agents considered for use in combination therapies include anastrozole (ARIMIDEX®), bicalutamide (CASODEX®), bleomycin sulfate (BLENOXANE®), busulfan (MYLERAN®), busulfan injection (BUSULFEX®), capecitabine (XELODA®), N4-pentoxycarbonyl-5-deoxy-5-fluorocytidine, carboplatin (PARAPLATIN®), carmustine (BICNU®), chlorambucil (LEUKERAN®), cisplatin (PLATINOL®), cladribine (LEUSTATIN®), cyclophosphamide (CYTOXAN® or NEOSAR®), cytarabine, cytosine arabinoside (CYTOSAR-U®), cytarabine liposome injection (DEPOCYT®), dacarbazine (DTIC-DOME®), dactinomycin (Actinomycin D, Cosmegan), daunorubicin hydrochloride (CERUBIDINE®), daunorubicin citrate liposome injection (DAUNOXOME®), dexamethasone, docetaxel (TAXOTERE®), doxorubicin hydrochloride (ADRIAMYCIN®, RUBEX®), etoposide (VEPESID®), fludarabine phosphate (FLUDARA®), 5-fluorouracil (ADRUCIL®, EFUDEX®), flutamide (EULEXIN®), tezacitibine, Gemcitabine (difluorodeoxycitidine), hydroxyurea (HYDREA®), Idarubicin (IDAMYCIN®), ifosfamide (IFEX®), irinotecan (CAMPTOSAR®), L-asparaginase (ELSPAR®), leucovorin calcium, melphalan (ALKERAN®), 6-mercaptopurine (PURINETHOL®), methotrexate (FOLEX®), mitoxantrone (NOVANTRONE®), mylotarg, paclitaxel (TAXOL®), phoenix (Yttrium90/MX-DTPA), pentostatin, polifeprosan 20 with carmustine implant (GLIADEL®), tamoxifen citrate (NOLVADEX®), teniposide (VUMON®), 6-thioguanine, thiotepa, tirapazamine (TIRAZONE®), topotecan hydrochloride for injection (HYCAMPTIN®), vinblastine (VELBAN®), vincristine (ONCOVIN®), and vinorelbine (NAVELBINE®). Exemplary alkylating agents include, without limitation, nitrogen mustards, ethylenimine derivatives, alkyl sulfonates, nitrosoureas and triazenes): uracil mustard (AMINOURACIL MUSTARD®, CHLORETHAMINACIL®, DEMETHYLDOPAN®, DESMETHYLDOPAN®, HAEMANTHAMINE®, NORDOPAN®, URACIL NITROGEN MUSTARD®, URACILLOST®, URACILMOSTAZA®, URAMUSTIN®, URAMUSTINE®), chlormethine (MUSTARGEN®), cyclophosphamide (CYTOXAN®, NEOSAR®, CLAFEN®, ENDOXAN®, PROCYTOX®, REVIMMUNE™), ifosfamide (MITOXANA®), melphalan (ALKERAN®), Chlorambucil (LEUKERAN®), pipobroman (AMEDEL®, VERCYTE®), triethylenemelamine (HEMEL®, HEXYLEN®, HEXASTAT®), triethylenethiophosphoramine, Temozolomide (TEMODAR®), thiotepa (THIOPLEX®), busulfan (BUSILVEX®, MYLERAN®), carmustine (BICNU®), lomustine (CEENU®), streptozocin (ZANOSAR®), and Dacarbazine (DTIC-DOME®). Additional exemplary alkylating agents include, without limitation, Oxaliplatin (ELOXATIN®); Temozolomide (TEMODAR® and TEMODAL®); Dactinomycin (also known as actinomycin-D, COSMEGEN®); Melphalan (also known as L-PAM, L-sarcolysin, and phenylalanine mustard, ALKERAN®); Altretamine (also known as hexamethylmelamine (HMM), HEXYLEN®); Carmustine (BICNU®); Bendamustine (TREANDA®); Busulfan (BUSULFEX® and MYLERAN®); Carboplatin (PARAPLATIN®); Lomustine (also known as CCNU, CEENU®); Cisplatin (also known as CDDP, PLATINOL® and PLATINOL®-AQ); Chlorambucil (LEUKERAN®); Cyclophosphamide (CYTOXAN® and NEOSAR®); Dacarbazine (also known as DTIC, DIC and imidazole carboxamide, DTIC-DOME®); Altretamine (also known as hexamethylmelamine (HMM), HEXYLEN®); Ifosfamide (IFEX®); Prednumustine; Procarbazine (MATULANE®); Mechlorethamine (also known as nitrogen mustard, mustine and mechloroethamine hydrochloride, MUSTARGEN®); Streptozocin (ZANOSAR®); Thiotepa (also known as thiophosphoamide, TESPA and TSPA, THIOPLEX®); Cyclophosphamide (ENDOXAN®, CYTOXAN®, NEOSAR®, PROCYTOX®, REVIMMUNE®); and Bendamustine HCl (TREANDA®). Exemplary mTOR inhibitors include, e.g., temsirolimus; ridaforolimus (formally known as deferolimus, (1R,2R,4S)-4-[(2R)-2[(1R,9S,12S, 15R, 16E, 18R, 19R,21R, 23S,24E,26E,28Z,30S,32S,35R)-1,18-dihydroxy-19,30-dimethoxy-15,17,21,23, 29,35-hexamethyl-2,3, 10, 14,20-pentaoxo-11,36-dioxa-4-azatricyclo[ 30.3.1.04.9]hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (AFINITOR® or RAD001);

rapamycin (AY22989, SIROLIMUS®); simapimod (CAS164301-51-3); emsirolimus, (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-4-[[4-(4-oxo-8-phenyl-4H-1-benzopyran-2-yl)morpholinium-4-yl]methoxy ]butyl]-L-arginylglycyl-L-α-aspartylL-serine-, inner salt (SF1126, CAS 936487-67-1), and XL765. Exemplary immunomodulators include, e.g., afutuzumab (available from ROCHE®); pegfilgrastim (NEULASTA®); lenalidomide (CC-5013, REVLIMID®); thalidomide (THALOMID®), actimid (CC4047); and IRX-2 (mixture of human cytokines including interleukin 1, interleukin 2, and interferon γ, CAS 951209-71-5, available from IRX Therapeutics). Exemplary anthracyclines include, e.g., doxorubicin (Adriamycin® and RUBEX®); bleomycin (LENOXANE®); daunorubicin (dauorubicin hydrochloride, daunomycin, and rubidomycin hydrochloride, CERUBIDINE®); daunorubicin liposomal (daunorubicin citrate liposome, DAUNOXOME®); mitoxantrone (DHAD, NOVANTRONE®); epirubicin (ELLENCE™); idarubicin (IDAMYCIN®, IDAMYCIN PFS®); mitomycin C (MUTAMYCIN®); geldanamycin; herbimycin; ravidomycin; and desacetylravidomycin. Exemplary vinca alkaloids include, e.g., vinorelbine tartrate (NAVELBINE®), Vincristine (ONCOVIN®), and Vindesine (ELDISINE®)); vinblastine (also known as vinblastine sulfate, vincaleukoblastine and VLB, ALKABAN-AQ® and VELBAN®); and vinorelbine (NAVELBINE®). Exemplary proteosome inhibitors include bortezomib (VELCADE®); carfilzomib (PX-171-007, (S)-4-Methyl-N-((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)-pentanamide); marizomib (NPI-0052); ixazomib citrate (MLN-9708); delanzomib (CEP-18770); and O-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912). Exemplary GITR agonists include, e.g., GITR fusion proteins and anti-GITR antibodies (e.g., bivalent anti-GITR antibodies) such as, e.g., a GITR fusion protein described in U.S. Patent No. 6,111,090, European Patent No .: 090505B1, U.S. Pat. No. 8,586,023, PCT Publication Nos .: WO 2010/003118 and 2011/090754, or an anti-GITR antibody described, e.g., in U.S. Patent No. 7,025,962, European Patent No. 1947183B1, U.S. Pat. Nos. 7,812,135, 8,388,967, 8,591,886, European Patent No. EP 1866339, PCT Publication No. WO 2011/028683, PCT Publication No. WO 2013/039954, PCT Publication No. WO2005/007190, PCT Publication No. WO 2007/133822, PCT Publication No. WO2005/055808, PCT Publication No. WO 1999/40196, PCT Publication No. WO 2001/03720, PCT Publication No. WO 1999/20758, PCT Publication No. WO2006/083289, PCT Publication No. WO 2005/115451, U.S. Pat. No. 7,618,632, and PCT Publication No. WO 2011/051726.

In some embodiments, the subject can be administered an agent which enhances the activity of a CAR-expressing cell. For example, in one embodiment, the agent can be an agent which inhibits an inhibitory molecule. Inhibitory molecules, e.g., Programmed Death 1 (PD-1), can, in some embodiments, decrease the ability of a CAR-expressing cell to mount an immune effector response. Examples of inhibitory molecules include PD-1, PD-L1, CTLA4, TIM-3, LAG3, VISTA, BTLA, TIGIT, LAIR1, CD160, 2B4 and TGFR-beta. Inhibition of an inhibitory molecule, e.g., by inhibition at the DNA, RNA or protein level, can optimize a CAR-expressing cell performance. In embodiments, an inhibitory nucleic acid, e.g., an inhibitory nucleic acid, e.g., a dsRNA, e.g., a siRNA or shRNA, can be used to inhibit expression of an inhibitory molecule in the CAR-expressing cell. In an embodiment the inhibitor is a shRNA. In an embodiment, the inhibitory molecule is inhibited within a CAR-expressing cell. In these embodiments, a dsRNA molecule that inhibits expression of the inhibitory molecule is linked to the nucleic acid that encodes a component, e.g., all of the components, of the CAR. In one embodiment, the inhibitor of an inhibitory signal can be, e.g., an antibody or antibody fragment that binds to an inhibitory molecule. For example, the agent can be an antibody or antibody fragment that binds to PD-1, PD-L1, PD-L2 or CTLA4 (e.g., ipilimumab (also referred to as MDX-010 and MDX-101, and marketed as YERVOY®; Bristol-Myers Squibb; Tremelimumab (IgG2 monoclonal antibody available from Pfizer, formerly known as ticilimumab, CP-675,206).). In an embodiment, the agent is an antibody or antibody fragment that binds to LAG3.

Programmed Death (PD)-1 is an inhibitory member of the CD28 family of receptors that also includes CD28, CTLA-4, ICOS, and BTLA. PD-1 is expressed on activated B cells, T cells and myeloid cells (Agata et al. 1996 Int. Immunol 8: 765-75). Two ligands for PD-1, PD-L1 and PD-L2 have been shown to downregulate T cell activation upon binding to PD-1 (Freeman et al. 2000 J Exp Med 192: 1027-34; Latchman et al. 2001 Nat Immunol 2:261-8; Carter et al. 2002 Eur J Immunol 32:634-43). PD-L1 is abundant in human cancers (Dong et al. 2003 J Mol Med 81: 281-7; Blank et al. 2005 Cancer Immunol. Immunother 54: 307-314; Konishi et al. 2004 Clin Cancer Res 10: 5094). Immune suppression can be reversed by inhibiting the local interaction of PD-1 with PD-L1. Antibodies, antigen binding molecules, and other inhibitors of PD-1, PD-L1 and PD-L2 are available in the art and may be used combination with a CAR described herein. For example, nivolumab (also referred to as BMS-936558 or MDX_{1106;} Bristol-Myers Squibb) is a fully human IgG4 monoclonal antibody which specifically blocks PD-1. Nivolumab (clone 5C4) and other human monoclonal antibodies that specifically bind to PD-1 are disclosed in U.S. Pat. No. 8,008,449 and PCT Publication No. WO2006/121168. Pidilizumab (CT-011; Cure Tech) is a humanized IgGlk monoclonal antibody that binds to PD-1. Pidilizumab and other humanized anti-PD-1 monoclonal antibodies are disclosed in PCT Publication No. WO2009/101611. Lambrolizumab (also referred to as MK03475; Merck) is a humanized IgG₄ monoclonal antibody that binds to PD-1. Lambrolizumab and other humanized anti-PD-1 antibodies are disclosed in U.S. Pat. No. 8,354,509 and PCT Publication No. WO2009/114335. MDPL3280A (Genentech/Roche) is a human Fc optimized IgG₁ monoclonal antibody that binds to PD-L1. MDPL3280A and other human monoclonal antibodies to PD-L1 are disclosed in U.S. Pat. No. 7,943,743 and U.S. Publication No. 2012/0039906. Other anti-PD-L1 binding agents include YW243.55.S70 (heavy and light chain variable regions are shown in SEQ ID NOs: 20 and 21 in PCT Publication No. WO2010/077634) and MDX-1 105 (also referred to as BMS-936559, and, e.g., anti-PD-L1 binding agents disclosed in PCT Publication No. WO2007/005874). AMP-224 (B7-DCIg; Amplimmune; e.g., disclosed in PCT Publication No. WO2010/027827 and PCT Publication No. WO2011/066342), is a PD-L2 Fc fusion soluble receptor that blocks the interaction between PD-1 and B7-H1. Other anti-PD-1 antibodies include AMP 514 (Amplimmune), among others, e.g., anti-PD-1 antibodies disclosed in U.S. Pat. No. 8,609,089, U.S. Publication No. 2010/028330, and/or U.S. Publication No. 2012/0114649.

### Kits

Kits are also provided. For example, kits for treating a subject with a tumor that expresses a tumor antigen, such as, but not limited to, CD22, CD123, CEA, EGFRVIII, ErbB2, HER2, IL-13ralpha2, MUC1, CD19 or CD20. The kits will typically include a disclosed nucleic acid encoding a CAR, T cell expressing a CA or compositions including such molecules.

The kit can include a container and a label or package insert on or associated with the container. Suitable containers include, for example, bottles, vials, syringes, etc. The containers may be formed from a variety of materials such as glass or plastic. The container typically holds a composition including one or more of the disclosed CARs, nucleic acid encoding the CAR, or host cells. In several embodiments the container may have a sterile access port (for example the container may be an intravenous solution bag or a vial having a stopper pierceable by a hypodermic injection needle). A label or package insert indicates that the composition is used for treating the particular condition.

The label or package insert typically will further include instructions for use of the CAR, nucleic acid encoding the CAR, host cells, or compositions included in the kit. The package insert typically includes instructions customarily included in commercial packages of therapeutic products that contain information about the indications, usage, dosage, administration, contraindications and/or warnings concerning the use of such therapeutic products. The instructional materials may be written, in an electronic form (such as a computer diskette or compact disk) or may be visual (such as video files). The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art.

The disclosure is illustrated by the following non-limiting Examples.

### EXAMPLES

The use of engineered T cells expressing chimeric antigen receptors (CARs) directed at specific tumor antigens has gained significant traction in the treatment of specific hematological malignancies (5). The efficacy of this approach is also regulated by both the expression of endogenous positive and negative regulators in the CAR T cells, as well as the inclusion of different co-stimulatory signaling domains in the CAR itself.

The high expression of Tim-3 on “exhausted” CD8⁺ T cells observed under conditions of chronic infection or within the tumor microenvironment. These cells comprise a subset of the cells that express the immune checkpoint molecule PD-1, which was previously described as the most robust marker for exhausted T cells; indeed, monoclonal antibodies (mAb's) that interfere with PD-1 interactions with its ligands (PD-L1, PD-L2) have demonstrated efficacy in a subset of cancer patients. Interestingly, T cells expressing high levels of both PD-1 and Tim-3 appear to be even more dysfunctional than those expressing PD-1 alone, as originally shown in chronic viral infection (Fourcade et al., J Exp Med 207, 2175-2186 (2010); Jin et al., Proc Natl Acad Sci U S A 107, 14733-14738 (2010); Kassu et al., J Immunol 185, 3007-3018 (2010); Vali et al., Eur J Immunol 40, 2493-2505 (2010)). Consistent with this notion, dual blockade of PD-1 and Tim-3 has a modestly enhanced ability to “rescue” the function of a population of exhausted T cells (Fourcade et al., supra, 2010; Sakusishi et al., J Exp Med 207, 2187-2194 (2010)). Several antibodies directed against Tim-3 are now in clinical trials and combination therapies are being actively explored (He et al., Onco Targets Ther 11, 7005-7009 (2018)). Thus, a prominent model that has emerged is that Tim-3 primarily functions as a negative regulator of T cell activation, in a manner similar to PD-1.

It is disclosed herein that there is robust recruitment of Tim-3 to the immune synapse (IS) lags recruitment of the TCR/CD3 complex to the IS. Furthermore, IS recruitment of Tim-3 is mainly regulated by its transmembrane (TM) domain. By substituting the TM, it was shown that IS recruitment of Tim-3 correlates with the co-stimulatory activity of the protein. In addition, even under conditions of enforced IS recruitment with a chimeric antigen receptor (CAR), the cytoplasmic domain of Tim-3 provides a co-stimulatory, but not inhibitory, signal for T cell activation. Thus, it was determined that the primary proximal effect of Tim-3 signal is to support T cell activation. It was demonstrated that the cytoplasmic domain of Tim-3 can function in a CAR.

### Example 1

Co-Stimulation of T cell Activation by a Chimeric Antigen Receptor Containing TIM-3

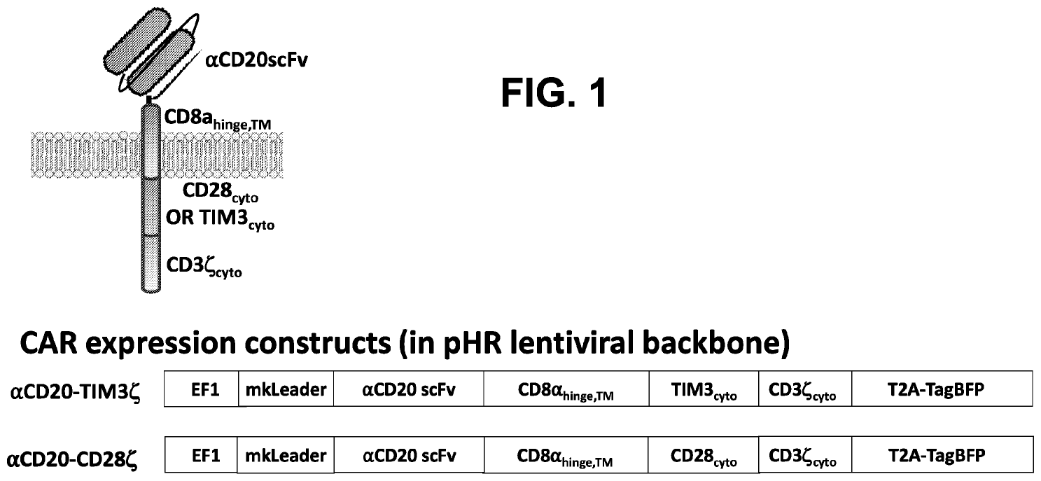

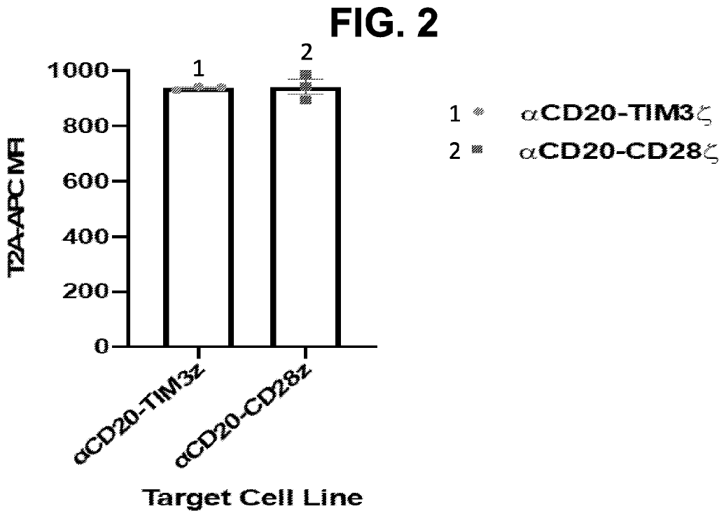

Previous immune synapse experiments were conducted in the absence of ligands to the ecto domain of TIM-3. It was determined if ligand engagement of TIM-3 could affect its ability to localize to the IS and/or provide a co-stimulatory signal, as in the case of CD28, for example (Bromley et al., Nat Immunol 2: 1159-1166, 2001; Pentcheva-Hoang et al., Immunity 21: 401-413, 2004). While several ligands for TIM-3 have been described, including galectin-9, HMGB1, PS and CEACAM1, none of these is specific to TIM-3 (Ferris et al., J Immunol 193: 1525-1530, 2014). The effects of enforced localization of TIM-3 to the immune synapse was tested, using a chimeric antigen receptor (CAR) approach. The activity of an anti-CD20 CAR containing the cytoplasmic domains of the TCR ζ protein and CD28 were compared with one containing ζ and the cytoplasmic domain of TIM-3 (FIG. 1). These constructs were expressed at equivalent levels on the surface of transduced Jurkat T cells (FIG. 2).

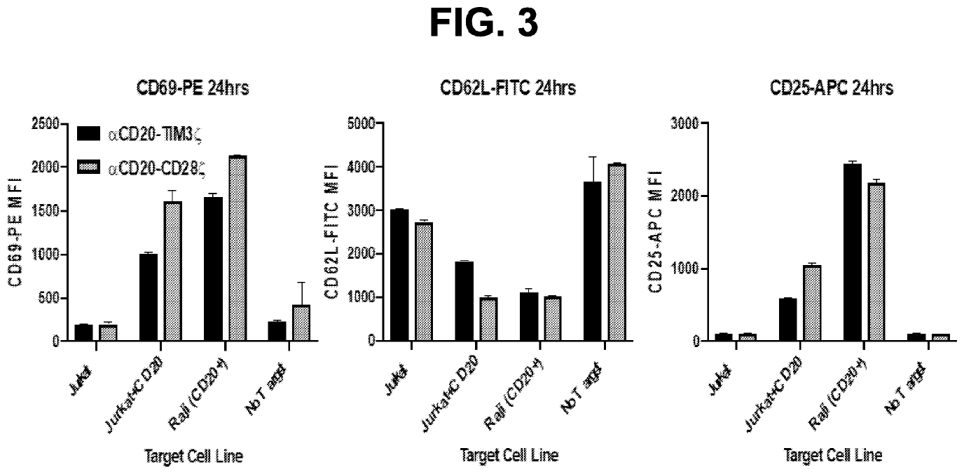

The functional consequences of this enforced localization of the TIM-3 cytoplasmic tail to the immune synapse was tested in the context of a CAR. Jurkat T cells expressing either the CD28- or TIM-3-containing CAR were mixed with CD20-expressing Raji B cells as targets. Using this approach, it was found that the TIM-3-containing CAR was as efficient as a CD28-containing CAR at mediating upregulation of the early activation markers CD69 and CD25 (FIG. 3). This activity was dependent on the presence of the CD20 antigen on the target cells, as demonstrated by the effects of culturing CAR-expressing cells with either parental Jurkat cells or those expressing CD20 (FIG. 3).

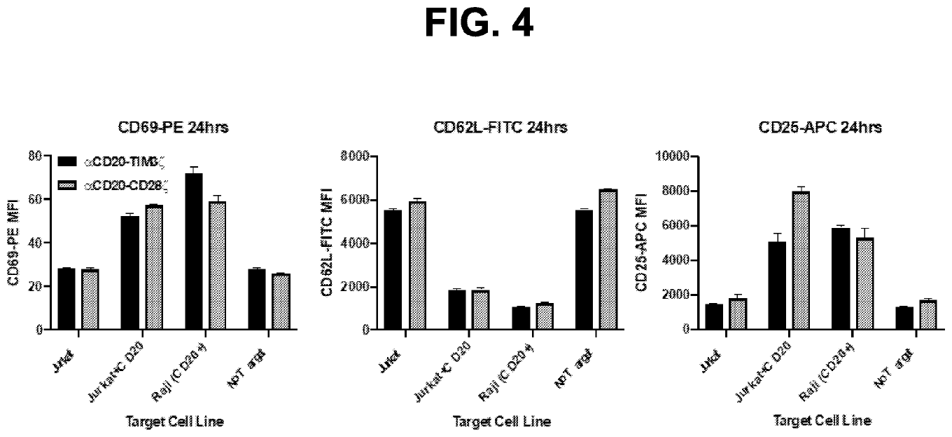

Since the above experiments were carried out with the transformed Jurkat T cell line, the results of these functional experiments were validated with primary human T cells. Peripheral blood T cells from normal human donors were infected with the same lentiviruses used to generate the Jurkat lines, after first stimulating the cells in vitro to make them permissive to infection. These CAR-T cells were mixed with the same target cells described above. Consistent with the Jurkat CAR-T results, the TIM-3-containing CAR mediated robust upregulation of both CD69 and CD25, to a degree comparable with the CD28-containing CAR (FIG. 4).

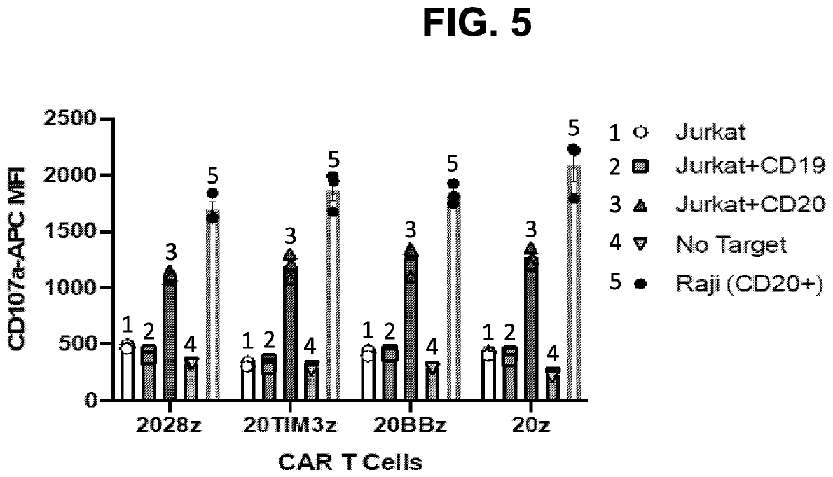

To more directly assess the functional effects of the above CARs, the ability of T cells expressing the CARs to undergo cytotoxic granule release was assessed in the presence of target cells carrying their target antigen. Cell-surface exposure of the protein CD107a (aka LAMP1) serves as a surrogate for such granule release, which is important for the function of cytotoxic T cells. As shown in FIG. 5, human primary T cells expressing the indicated CAR constructs (X axis) were all able to mediate an increase in cell-surface CD107a after incubation only with target cells expressing CD20.

### Example 2

### Cellular and Biochemical Correlates of Function

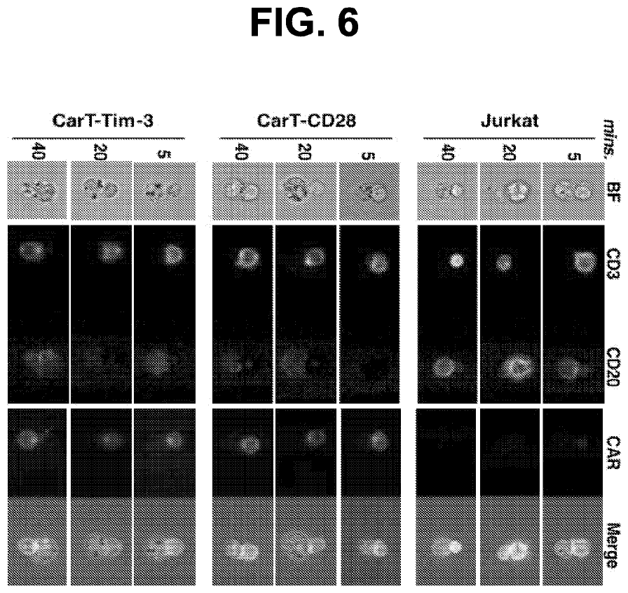

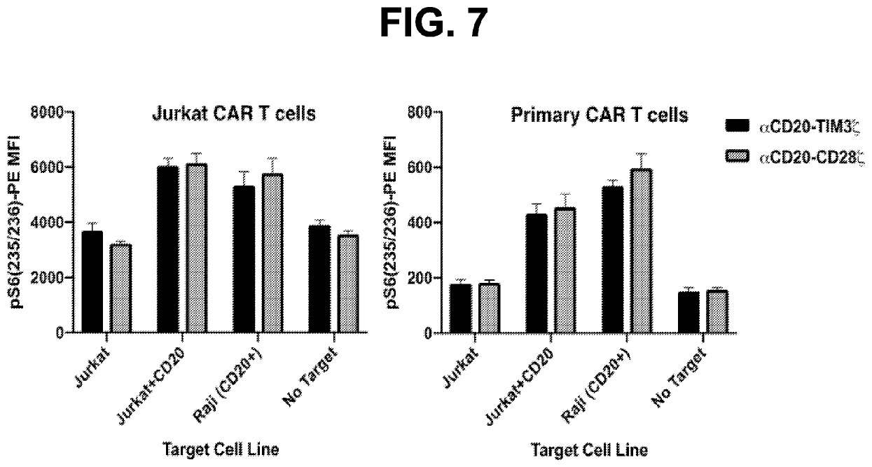

The receptors were expressed in Jurkat T cell s, which were cultured with CD20-expressing Raji B cells to generate conjugates, as in the above experiments. As expected, both of these CARs formed discrete immune synapses when bound to cells expressing the cognate antigen (FIG. 6). TIM-3 is capable of co-stimulating TCR-mediated phosphorylation of the ribosomal S6 protein, determined whether this activity is retained in the TIM-3 CAR. Indeed, consistent with the functional data, the TIM-3-containing CAR mediated robust pS6 upregulation, similar to the CD28 CAR, in both Jurkat T cells and primary human T cells (FIG. 7). Thus, in the context of a synthetic antigen receptor, a setting where the potential confounding effects of presence (or absence) of TIM-3 ligands, the cytoplasmic tail of TIM-3 still is permissive for robust T cell activation.

### Example 3

### General Applicability to CARs With Distinct Antigenic Targets

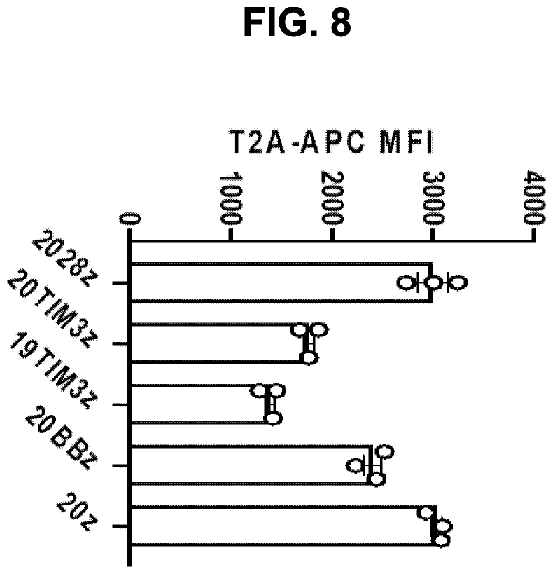

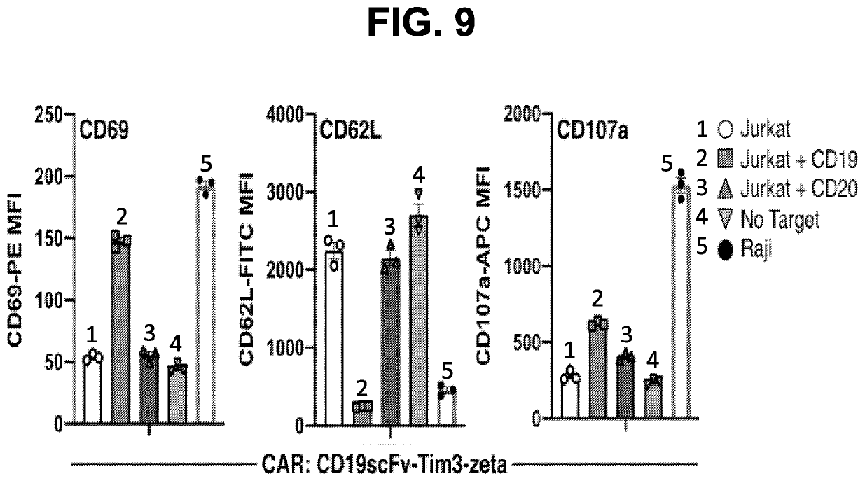

The functional impact of including the cytoplasmic tail of TIM-3, along with TCR zeta, was tested in the context of a CAR targeting a different antigen. As shown in FIG. 8, a CD19-targeted TIM-3-zeta CAR was expressed equivalently to the CD20 TIM-3-zeta CAR, and only slightly lower than CARs containing CD28, 41BB or zeta alone. At a functional level, the CD19-specific CAR containing TIM-3 was also able to mediate the activation of primary T cells transduced with the CAR and cultured with CD19-expressing target cells (FIG. 9). Thus, the rather unexpected co-stimulatory activity of TIM-3 is not limited to a CD20 CAR.

The region of the TIM-3 cytoplasmic tail that is responsible for the above-described T cell co-stimulatory activity by this molecule was identified (see FIG. 10). Deletion of part of the TIM-3 cytoplasmic tail (“T1” construct), which removed the three C-terminal most tyrosine residues, resulted in no decrease in TIM-3 co-stimulatory activity. In fact, in some assays, this construct provided even more co-stimulatory activity, compared with full-length TIM-3. By contrast, a more severe deletion (“T2”), which in addition deleted tyrosines at positions 256 and 263 (mouse TIM-3 numbering) led to a complete abolition of the co-stimulatory activity in T cells (22). TIM-3 CARS can be generated carrying these deletion mutations in TIM-3. In addition, constructs can be produced with single or double point mutations in Y256 and/or Y263, to confirm the roles of these residues, which can be phosphorylated in some contexts (22)).

The T2 truncation will eliminate the TIM-3 co-stimulatory activity of the CAR containing it but should not affect the basal activity mediated by TCR zeta. The Tl truncation, on the other hand, many yield a CAR (along with TCR zeta) that possesses even more activity than the WT TIM-3 CAR.

The TIM-3 CARs are characterized in comparison with CD28 and 4-1BB CARs in vitro for differences in T cell proliferation, target cell lysis, mitochondrial function, and cytokine production. In vivo characterization is performed in a human xenograft tumor model assaying for effects of TIM-3 CAR T cells on tumor growth as well as CAR T cell persistence in the animal as well as functionality in re-challenge experiments in vitro after re-isolating the cells from the animal.

### Example 4

### Materials and Methods for Examples 1-3

### Construction of Chimeras

To generate the TIM-3 CAR constructs (pHR-EF1-aCD19-TIM-3z-T2A-TagBFP & pHR-EF1-aCD20-TIM-3z-T2A-TagBFP), DNA encoding the human TIM-3 cytoplasmic domain was codon optimized, synthesized (Integrated DNA Technologies), and then cloned into the pHR-EF1-aCD19-28z or pHR-EF1-aCD20-28z CAR lentiviral expression vectors, respectively, to replace the CD28 co-signaling domain using isothermal assembly.

### Generation of Virus and Infection of Cells

To generate virus, HEK293T cells (ATCC) were transfected with pVSV-G (VSV glycoprotein expression plasmid), pMD2.G and the CAR-expression plasmid via calcium phosphate transfection (Clontech). At 16 hours post-transfection cells were washed with PBS and fed with complete DMEM media containing 6 mM sodium butyrate (Sigma Aldrich). Supernatants were collected at 24 and 48 hrs and were subsequently combined and filtered through a 45 μm vacuum filter. Viral particles were concentrated by ultracentrifugation for 1.5 hours at 24,500 rpm, and viral pellets were re-suspended in 0.05 ml of supplemented RPMI medium and frozen at −80° C.

CAR T Cell and Target Cell Co-Incubation Assays for pS6 Activation 100,000 Jurkat or Primary T cells expressing the TIM-3 or CD28 CAR were incubated with an equal number of the indicated antigen positive or negative target cell line in a V-bottom plate for 30 minutes at 37C in 5% CO2. Cells then underwent intracellular staining and flow cytometry as detailed below.

### CAR T Cell and Target Cell Co-Incubation Assays for Activation of T Cell Signaling Markers

100,000 Jurkat or Primary T cells expressing the TIM-3 or CD28 CAR were incubated with an equal number of the indicated antigen positive or negative target cell line in a V-bottom plate for 18 hours at 37C in 5% CO2. Cells then underwent cell-surface antibody staining and flow cytometry as detailed below.

### Flow Cytometry

For quantification of cell surface markers (CD69, CD62L, CD25, CD107a) cells were stained using the recommended dose of antibody diluted in flow cytometry buffer (PBS+2% FBS), for 30 minutes at 4° C. followed by two washes with flow cytometry buffer. For staining of intracellular markers (T2A, or pS6), cells were fixed and permeabilized using the Cytofix/Cytoperm kit (BD Biosciences) according to manufacturer's protocol, and subsequently underwent antibody staining and washing as above. Samples were evaluated on a BD Fortessa cytometer. Activation marker expression was specifically assessed for CAR+ cells (vs. target cells) by gating on the TagBFP positive cell population for cell surface stained cells, or for T2A-expressing cells for the pS6 assay.

### ImageStream Imaging (T Cell Synapse Analysis)

Jurkat-Raji cell conjugates were fixed with 2% paraformaldehyde and then stained with antibodies at 4° C. for extracellular staining. Imaging cytometry data on fixed and stained cells were acquired on an AMNIS® IMAGESTREAMX® Mark II running INSPIRE® software (Millipore Sigma). Analysis was performed using IDEAS® software (Millipore Sigma). First, focused cells, based on the gradient root mean square feature, were identified. Doublets were identified by the aspect ratio and area of bright field and then gated by the aspect ratio and staining intensity of B cells. Gated cells were further refined by the aspect ratio and staining intensity of T cells to identify T-B doublets. An interface mask covering the immunological synapse was made from brightfield and Raji staining. The percentages of CD3 and CAR recruitment were quantified from the ratio of the intensity of either CD3 or CAR in the interface mask to that of the whole cell mask. The intensity of pS6 in Jurkat T cells was quantified in the mask made by subtracting the B cell mask from the brightfield mask. For primary T-B conjugates, an interface mask was made from brightfield and CD8 staining. The intensity of pS6 in CD8+ T cells was quantified in the mask made from CD3 staining. Approximately 100-300 cells are represented in the Jurkat T-Raji conjugate data, and 100-2,000 cells are represented in the primary T-B conjugate data.

### Example 5

### Assessment of Tim-3 Recruitment to the IS by AMNIS® IMAGESTREAM® Analysis

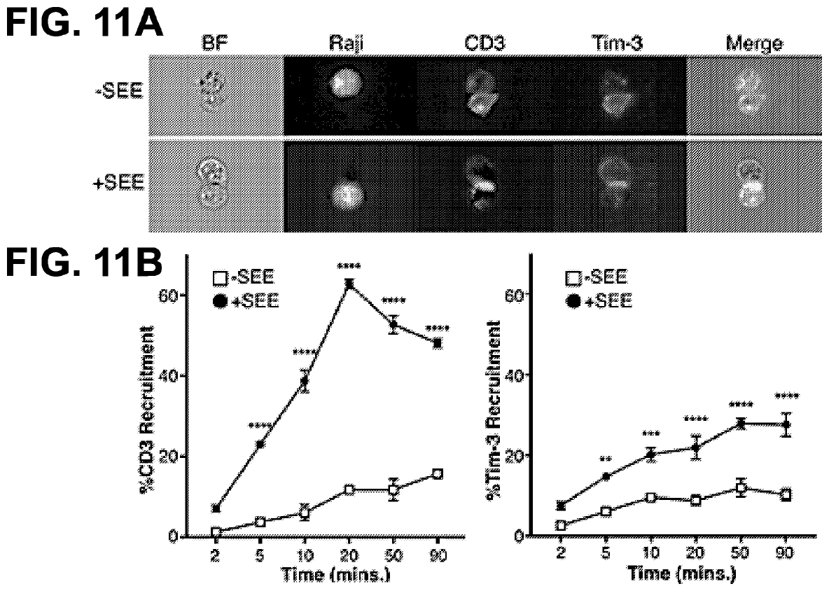

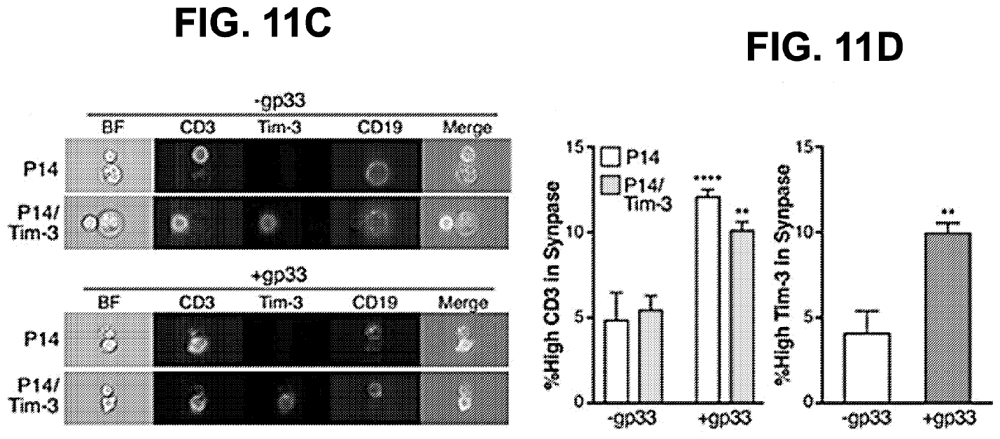

Co-stimulatory and co-inhibitory receptors often colocalize with TCR at the immunological synapse (IS) (Chen et al., Nat Rev Immunol 13, 227-242 (2013)). Tim-3 may be recruited to the IS in activated T cells (Clayton et al., J Immunol 192, 782-791 (2014)). To better understand the mechanisms underlying co-stimulation by Tim-3 in T cells, Tim-3 localization was examined during IS formation on Jurkat T cells transfected with Flag-tagged Tim-3, using the AMNIS® IMAGESTREAM® flow cytometry system. Raji cells (a human B cell lymphoma line) pulsed with SEE were used as APCs, and IS formation was confirmed on the basis of CD3 recruitment to the Jurkat T cell-Raji cell interface (FIGS. 11A-11B). An increase in the proportion of Tim-3 recruitment to the IS was observed over time, although Tim-3 displayed a lower overall degree of recruitment, with somewhat slower kinetics, compared with the behavior of CD3. To determine whether the same pattern of recruitment of Tim-3 is also seen in primary murine T cells, P14 TCR transgenic mice were employed, with or without expression of Tim-3 from a Rosa26 knock-in cassette (Avery et al, Proc Natl Acad Sci U S A 115, 2455-2460 (2018)). CD8⁺ T cells from these mice were stimulated with autologous T cell-depleted splenocytes, with or without the cognate peptide antigen, the gp33 peptide from LCMV. Thus, consistent with the results obtained with Jurkat T cells, enhanced recruitment of Tim-3 to the IS of CD8⁺ P14 TCR Tg x Tim-3 Tg T cells was observed in the presence of cognate peptide (FIGS. 11C-D).

### Example 6

### The Extracellular and Intracellular Domains of Tim-3 are Dispensable for its Recruitment to the Immune Synapse

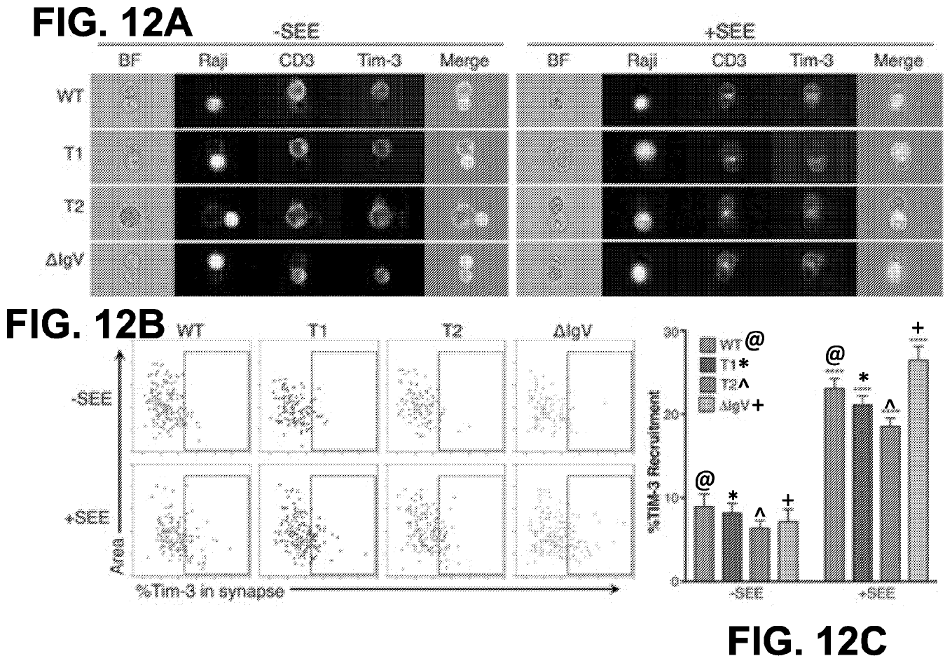

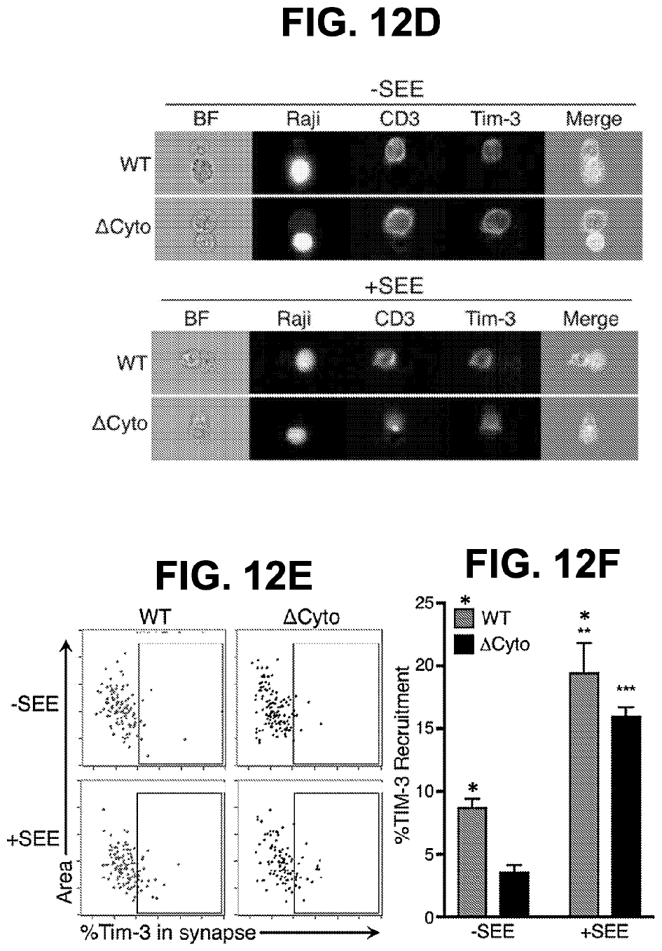

The roles were determined for specific Tim-3 domains in its recruitment to the IS. Using various mutants of Tim-3, it was shown that the cytoplasmic tail of Tim-3 is required for its co-stimulatory activity, at least at the level of anti-TCR-induced activation of NFAT/AP-1 and NF-κB transcriptional reporters (Lee et al., Mol Cell Biol 31, 3963-3974 (2011)). To address the relationship between Tim-3 signaling and its localization, Flag-tagged Tim-3 variants lacking either the sequence encompassing three tyrosines at positions 271, 272 and 274 (truncation 1; T1) or a more severely truncated variant also lacking tyrosines 256 and 263 (truncation 2; T2) were used. It was shown that the T1 construct retains Tim-3 co-stimulation of NFAT/AP-1 activity, while the T2 construct abolishes this activity (Lee et al., supra, 2011). Consistent with those data, it was found that the T1 mutant still retained the ability to enhance pS6 comparably to WT Tim-3, whereas the T2 truncation abolished the ability of Tim-3 to enhance pS6. Surprisingly, however, there was no difference in the localization of these constructs to the IS upon stimulation with SEE-pulsed Raji cells, compared to full-length Tim-3 (FIGS. 12A-12C). The T2 construct still retains 38 amino acids downstream of the transmembrane domain. To more rigorously address the role of the intracellular domain in Tim-3 IS recruitment, a Flag-tagged Tim-3 variant was used, lacking the entire cytoplasmic region (ΔCyto). However, there was no significant difference in the localization of the ΔCyto construct, compared to full-length Tim-3 (FIGS. 12D-12E). Thus, IS recruitment of Tim-3 does not require its cytoplasmic domain.

The Tim-3 IgV domain has been shown to bind to various and diverse ligands such as galectin-9, PS, HMGB1 and Ceacam-1 (Huang et al., Nature 517, 386-390 (2015); Zhu et al., Nat Immunol 6, 1245-1252 (2005); Chiba et al., Nat Immunol 13, 832-842 (2012); DeKruyff et al., J Immunol 184, 1918-1930 (2010)), which constitute all known Tim-3 ligands. Therefore, a Flag-tagged Tim-3 variant lacking the IgV domain (AlgV) was used, to address the role of ligand binding in Tim-3 IS recruitment. As with the cytoplasmic tail deletions, deletion of the IgV domain did not impair Tim-3 recruitment (FIG. 12A-C), indicating that Tim-3 recruitment to the IS may not require ligand binding, since deletion of the IgV domain eliminates the ability of Tim-3 to bind to all of its known ligands.

### Example 7

### The Transmembrane Domain of Tim-3 Regulates its Co-Stimulatory Activity and Recruitment to the Immune Synapse

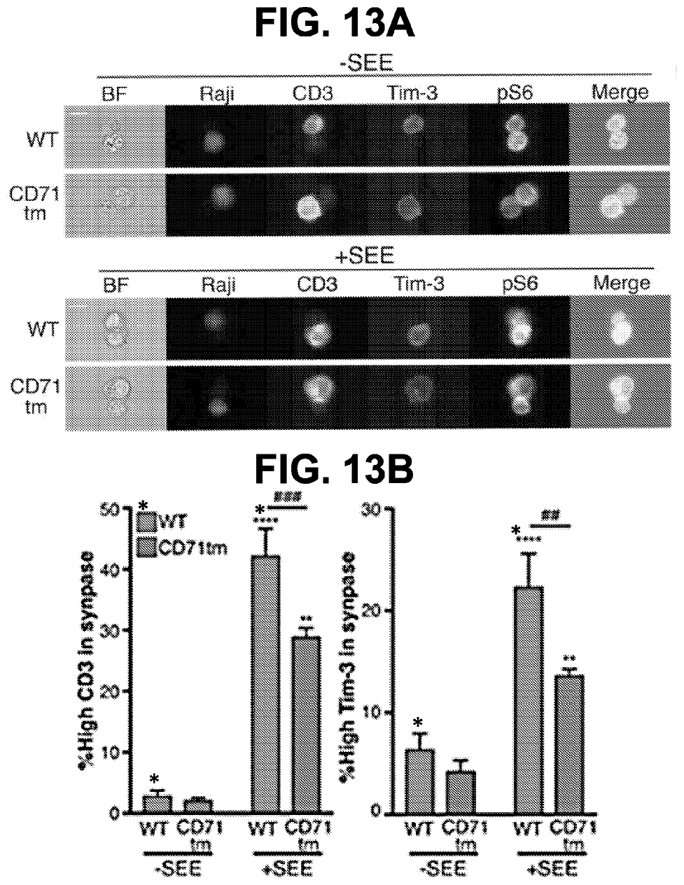

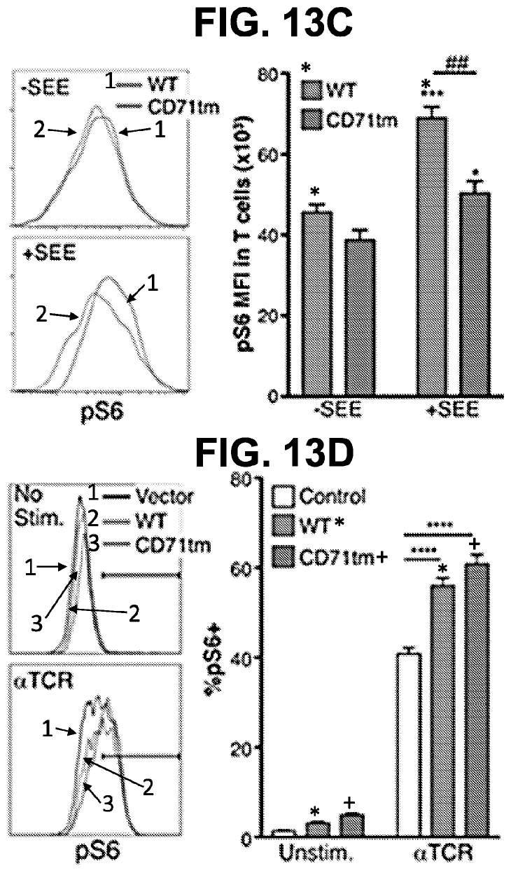

The transmembrane domain of some cell-surface molecules can regulate recruitment to lipid rafts, cholesterol-rich membrane sub-regions that accumulate at the IS. The role of the transmembrane domain of Tim-3 in IS recruitment was examined, using a Tim-3-CD71 transmembrane chimeric protein (CD71tm), in which the transmembrane domain of Tim-3 was replaced with the corresponding domain of CD71. The CD71 TM domain has been shown to mediate exclusion from lipid rafts in the context of a chimeric receptor (Cho et al., J Virol 80, 108-118 (2006)). Indeed, a decrease was observed in Tim-3 localization at the IS upon stimulation with SEE-pulsed Raji cells, in Jurkat T cells transfected with CD71tm Tim-3, compared with WT Tim-3 (FIGS. 13A-13B). Thus, the transmembrane domain of Tim-3 is important for its localization to the IS.

To assess the role of the transmembrane domain of Tim-3 in T cell activation during IS formation, transfected Jurkat T cells were stimulated with Raji cells pulsed with SEE. It was observed that Jurkat T cells transfected with WT Tim-3 showed higher pS6 levels than cells transfected with CD71tm Tim-3, after conjugation with SEE-pulsed Raji cells (FIG. 13C). To assess whether replacement of the transmembrane domain of Tim-3 affects T cell activation more generally, Jurkat T cells expressing either Flag-tagged WT Tim-3 or CD71tm Tim-3 were stimulated with alpha-TCR mAb. However, in this context, similar pS6 levels were found between cells expressing WT and CD71tm forms of Tim-3 (FIG. 13D), indicating that CD71tm Tim-3 retains the ability to enhance pS6 after antibody stimulation, which does not result in IS formation. These data suggest that IS localization of Tim-3 is required for the co-stimulatory effect of Tim-3 on T cell activation, specifically under conditions inducing IS formation.

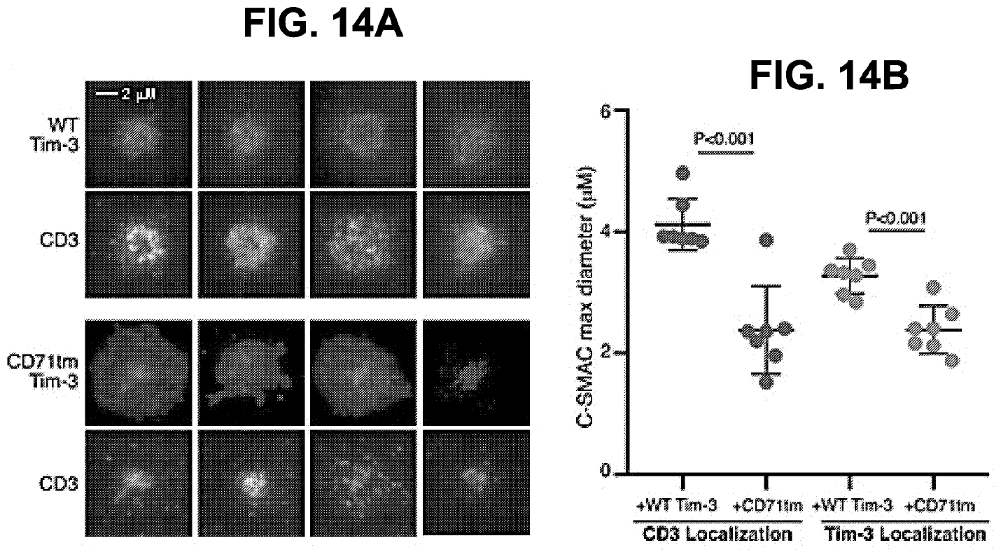

In the above experiments, the localization of Tim-3 and formation of immune synapse between T cells and APCs were assessed using imaging cytometry. This approach has the advantages of visualizing IS formation in a native T cell-APC setting as well as being amenable to high-throughput data collection. Tim-3 localization also was addressed in a system that allows for imaging of these structures at higher resolution and in real-time. The lipid bilayer system was used for this purpose (Dustin et al., “Supported planar bilayers for study of the immunological synapse,” in: Current protocols in immunology/edited by John E. Coligan et al., Chapter 18, Unit 18 13 (2007)), paired with TIRF microscopy and a fluorescence activating protein (FAP) chimeric approach, which together allowed for high signal:noise imaging of protein localization at the plasma membrane (Szent-Gyorgyl et al., Nat Biotechnol 26, 235-240 (2008)). Primary murine T cells were activated in vitro and transduced with lentivirus encoding WT or CD71tm Tim3-FAP fusion proteins (see schematic in FIG. 14A). Transduced cells were sorted and imaged on lipid bilayers containing ICAM-1 and anti-TCR beta Fab fragment. Consistent with results shown above recruitment of WT Tim-3 to the IS, as defined by TCR/CD3 recruitment (FIG. 14B), was observed.

The CD71tm variant of Tim-3 described above was imaged using the same system. Again, consistent with results obtained using APC conjugates, the CD71tm Tim-3 construct was recruited less efficiently to the IS formed on lipid bilayers (FIG. 14C). Most strikingly, cells expressing the CD71tm form of Tim-3 displayed significantly smaller immune synapses, as revealed by CD3 localization, suggesting a dominant inhibitory effect of this construct.

### Example 8

### Enforced IS Localization of the Tim-3 Cytoplasmic Tail is Still Permissive for T Cell Activation by a Chimeric Antigen Receptor

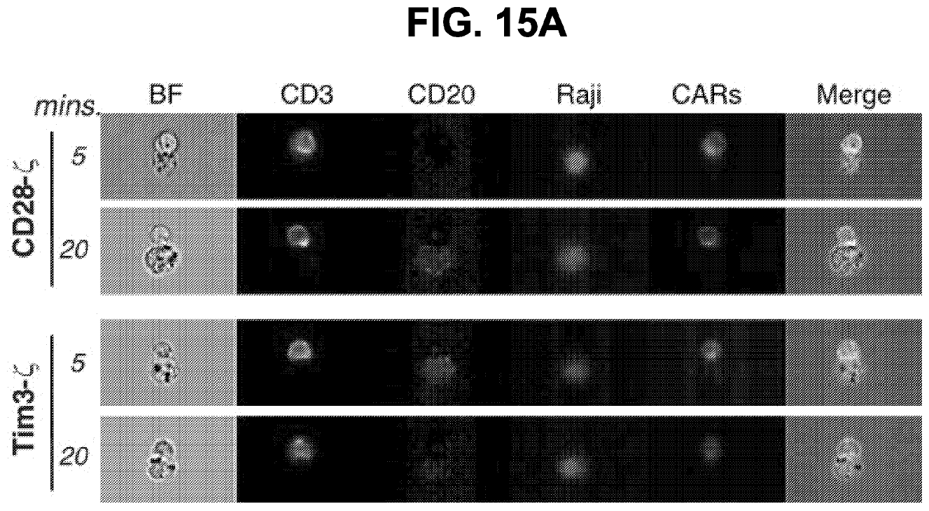

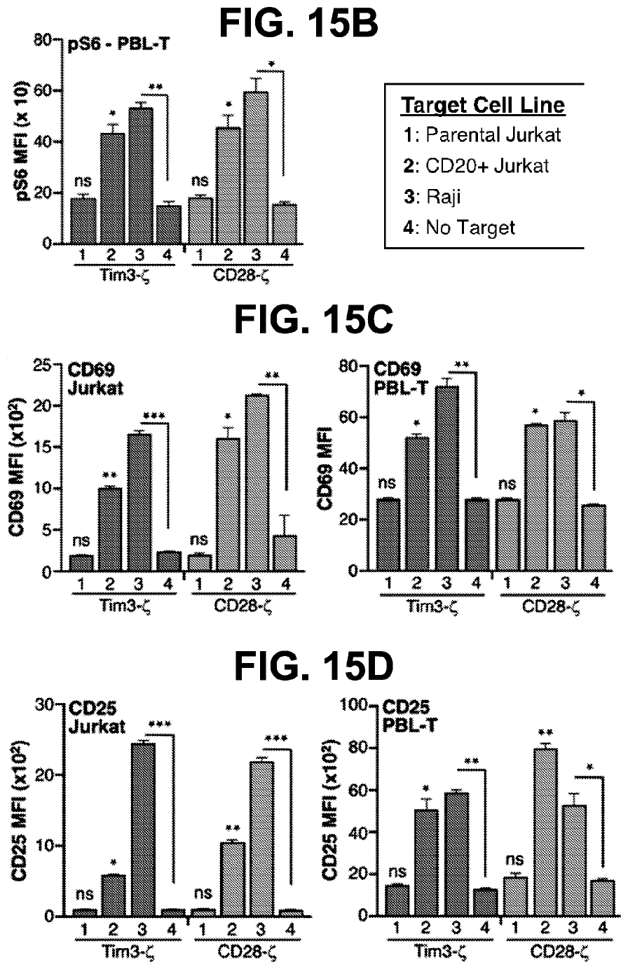

Multiple ligands have been reported to bind to the IgV domain of Tim-3, although none of these is exclusive to Tim-3. The studies in Examples 5-7 were conducted in the absence of added ligands to the ecto domain of Tim-3. The possibility was considered that ligand engagement of Tim-3 could affect its ability to localize to the IS and/or provide a co-stimulatory signal. While several ligands for Tim-3 have been described (Anderson et al., Immunity 44, 989-1004 (2016); Du et al., Int J Mol Sci 18, (2017)), including galectin-9, HMGB1, PS and CEACAM1, none of these is exclusive to Tim-3. The effects of enforced localization of Tim-3 to the IS was tested, using a chimeric antigen receptor (CAR) approach. The activity of an anti-CD20 CAR containing the cytoplasmic domains of TCR ζ and CD28 (Jensen et al., Biol Blood Marrow Transplant 4, 75-83 (1998); Maher et al., Nat Biotechnol 20, 70-75. (2002)) were compared, with one containing the cytoplasmic domains of ζ-Tim-3. Initially, these receptors were expressed in Jurkat T cells, which were cultured with CD20-expressing Raji B cells to generate conjugates, as in the above experiments. As expected, both of these CARs formed discrete IS's when bound to cells expressing cognate antigen (FIG. 15A).

The functional consequences of this enforced localization of the Tim-3 cytoplasmic tail to the IS was assessed. Jurkat T cells expressing either the CD28- or Tim3-containing CAR were mixed with Raji B cells (CD20+) as targets. Given the other findings (above) that Tim-3 is capable of co-stimulating TCR-mediated phosphorylation of the ribosomal S6 protein, it was determined whether this activity is retained in the Tim-3 CAR. Indeed, consistent with the functional data, the Tim-3 CAR mediated robust pS6 upregulation, similar to the function of the CD28 CAR, in both Jurkat T cells and primary human T cells (FIG. 15B). This was observed with two different target cell lines expressing CD20—either Raji cells (which express endogenous CD20) or Jurkat T cells transfected with CD20. Using this approach, it was found that the Tim3-containing CAR was roughly as efficient as a CD28-containing CAR at mediating upregulation of the early activation marker CD69 when the CARs were expressed in either Jurkat or primary human T cells (FIG. 15C). This activity was dependent on the presence of the CD20 antigen on target cells, since it was not observed in the absence of target cells or in the presence of parental Jurkat T cells, which do not express CD20. The effects of the two CARs was assessed on induced expression of CD25, which is upregulated with somewhat delayed kinetics, relative to CD69. Consistent with the effects shown above, the CD28 and Tim-3 CARs mediated antigen-specific upregulation of CD25 to a similar extent, in both Jurkat and primary human T cells (FIG. 15D). Thus, in the context of a synthetic antigen receptor, which eliminates potential confounding effects of Tim-3 ligands, the cytoplasmic tail of Tim-3 is permissive for robust T cell activation, similar to a standard CD28-containing CAR.

### Example 9

### Materials and Methods for Examples 5-8

Cell Lines: Jurkat and D10 T cell and Raji B cell lines were maintained in RPMI media supplemented with 10% bovine growth serum (BGS; Hyclone), penicillin, streptomycin, L-glutamine, non-essential amino acid, sodium pyruvate, HEPES and 2-mercaptoethanol. Recombinant human IL-2 (25 U/ml) was also supplemented for D10 cells. Human embryonic kidney (HEK) 293 cells were maintained in Dulbecco's MEM supplemented with 10% bovine growth serum (BGS), penicillin, streptomycin and L-glutamine.

Mice: FSF-Tim3 knock-in mice were generated as previously described (Avery et al., Proc Natl Acad Sci U S A 115, 2455-2460 (2018). P14 TCR transgenic mice were obtained from Jackson Laboratories. E8i-Cre mice are disclosed in Maekawa et al., Nat Immunol 9, 1140-1147 (2008). WT C57BL/6 mice were either obtained from Jackson Laboratories or bred in-house. Mice were age-matched within experiments.

Transfections and activation: Jurkat and D10 T cells were resuspended in RPMI media without supplements and electroporated with control plasmid, Flag-tagged WT Tim-3 or Flag-tagged mutant Tim-3 constructs, along with pMaxGFP plasmid in a Bio-Rad GenePulser at 260 V and 960 μF for Jurkat T cells and 250 V and 950 μF for D10 cells. Transfected cells were starved of serum in phosphate-buffered saline (PBS) with 0.1% bovine serum albumin (BSA) for 1 hr at 37° C. Cells were also pretreated with U0126 (Millipore Sigma, #662005) and/or Akti1/2 (Abcam, #ab142088) before being stimulated with the anti-Jurkat TCR Vβ8 monoclonal antibody C305. After stimulation, cells were washed in staining buffer (1% BGS-supplemented PBS) and incubated with anti-Flag-PE (clone L5; BIOLEGEND®, #637310) and GHOST DYE™ Violet 510 (Tonbo Biosciences, 13-0870-T100) at 4° C. for extracellular staining. Cells were then fixed and permeablized with the BD CYTOFIX™/CYTOPERM™ kit (BD Biosciences, #554714). Fixed and permeablized cells were then incubated at 4° C. with anti-phospho-S6 (Ser235/236) APC (clone D57.2.2E; Cell Signaling Technology, #14733). Samples were processed on a BD LSRII flow cytometer, and data were analyzed with FlowJo software.

Western blotting: Cells were lysed in ice-cold NP-40 lysis buffer (1% NP-40, 1 mM EDTA, 20 mM tris-HCL pH 7.4, 150 mM NaCl) with protease inhibitors. Proteins were separated by 10% SDS-polyacrylamide gel electrophoresis and were transferred onto polyvinylidene difluoride membranes which were then blocked in 4% BSA. The membranes were then incubated with the primary antibodies overnight. This was followed by incubating the membrane with HRP-conjugated secondary antibodies for 2hrs before detection with the SUPERSIGNAL® West Pico ECL substrate (Thermo Fisher Scientific, #34577) and imaging on a Protein Simple FluorChem M.

The following antibodies were used for Western blotting: Direct-Blot HRP anti-ERK1/2 Phospho (Thr202/Tyr204) (clone 4B11B69; BIOLEGEND®, #675505), anti-p44/42 MAPK (Erk 1/2) (CELL SIGNALING TECHNOLOGY®, #9102), anti-phospho-Akt (Thr308) (clone 244F9; CELL SIGNALING TECHNOLOGY®, #4056), anti-Akt (clone 2/PKBa/Akt; BD Biosciences, 610877)

Jurkat-Raji conjugate formation: Raji cells were labeled with either CELLTRACKER™ Orange CMRA Dye (Thermo Fisher Scientific, C34551) or Cell Proliferation Dye eFluor450 (Thermo Fisher Scientific, 65-0842-85) and then pulsed with 2 μg/ml staphylococcal enterotoxin E (SEE) (Toxin Technology, ET404) for 1 hr at 37° C. Transfected Jurkat T cells were mixed with Raji cells, either un-pulsed or pulsed with SEE, at a ratio of 1:1 and incubated for indicated times at 37° C.

Primary T-B cell conjugate formation: B cells from spleens and lymph node of naïve mice were purified by magnetic separation using a pan-B cell isolation kit (Miltenyi Biotec, #130-095-813), then incubated overnight with 30 μg/ml LPS (Sigma, #L2630) and 10 μM LCMV gp33-41 peptide (AnaSpec, #AS-61296). CD8 T cells from spleens and lymph nodes of either P14 or FSF-Tim-3/E8i-Cre/P14 mice were purified by magnetic separation using a naïve CD8⁺ T cell isolation kit (Miltenyi Biotec, #130-104-075) and then mixed with gp33-pulsed B cells at a ratio of 1:1 and incubated for indicated times at 37° C.

Imaging flow cytometry: Cell conjugates were fixed with 2% paraformaldehyde and then incubated with antibodies at 4° C. for extracellular staining. For intracellular staining, cells were permeabilized with 0.2% saponin and then incubated with anti-phospho-S6 (Ser235/236) antibody at 4° C.

Imaging cytometry data on fixed and stained cells were acquired on an AMNIS® IMAGESTREAMX® Mark II running INSPIRE® software (Millipore Sigma). Analysis was performed using IDEAS® software (Millipore Sigma). First, focused cells, based on the gradient root mean square feature, were identified. Doublets were identified by the aspect ratio and area of bright field and then gated by the aspect ratio and staining intensity of B cells. Gated cells were further refined by the aspect ratio and staining intensity of T cells to identify T-B cell doublets. For Jurkat T-Raji conjugates, an interface mask covering the immunological synapse was made from brightfield and Raji staining. The percentages of CD3 and Tim-3 recruitment were quantified from the ratio of the intensity of either CD3 or Tim-3 in the interface mask to that of the whole cell mask. The intensity of pS6 in Jurkat T cells was quantified in the mask made by subtracting the B cell mask from the brightfield mask. For primary T-B conjugates, an interface mask was made from brightfield and CD8 staining. The intensity of pS6 in CD8+ T cells was quantified in the mask made from CD3 staining. Approximately 100-300 cells are represented in the Jurkat T-Raji conjugate data, and 100-2,000 cells are represented in the primary T-B conjugate data.

The following antibodies were used for conjugation assay: anti-human CD3-FITC (clone OKT3; Tonbo Biosciences, 35-0037-T100), anti-Flag-APC (clone L5; BIOLEGEND®, #637308), anti-mouse CD3-FITC (clone 145-2C11; Tonbo Biosciences, 35-0031-U500), anti-mouse CD19-violetFluor450 (clone 1D3; Tonbo Biosciences, 75-0193-U100), anti-pS6 (Ser235/236) PE (clone D57.2.2E; CELL SIGNALING TECHNOLOGY®, #5316S).

TIRF imaging of T cell synapses on lipid bilayers: WT and CD71tm mutant Tim-3 constructs were cloned into MSCO-based retroviral vectors containing the fluorogen activating protein (FAP) sequence in-frame, followed by an IRES and coding sequence of mouse Thy1.1. constructs were then transfected into 293T cells, along with the pCL-Eco packaging plasmid, using TRANSIT® transfection reagent (Mirus).
CD8⁺ T cells were purified from C57BL/6 mice using a mouse CD8⁺ T cell isolation kit from Miltenyi. Cells were stimulated for 27 hrs on plates coated with anti-CD3/CD28 mAbs, plus 100 U/ml recombinant human IL-2. Activated T cells were harvested and transduced with FAP-encoding virus from the 293T cell transfectants. Transduced Thy 1.1⁺ cells were purified by sorting and kept on ice until imaging.

Lipid bilayers were constructed as described (Huppa et al., Nature 463, 963-967 (2010)), with some modifications (Kaizuka et al., Proc Natl Acad Sci U S A 104, 20296-20301 (2007).). Briefly, liposomes comprised of 90% dioleoylphosphocholine, 10% DOGS (1,2-dioleoyl-SN-Glycero-3-{[N(5-amino-1 carboxypentyl) iminodiacetic acid]succinyl}), and 0.2% biotin-CAP-PE (Avanti Polar Lipids) were deposited on glass coverslips cleaned with piranha solution (50:50 mixture of 30% H₂O₂ and 96% H₂SO₄). ALEXA FLUOR-488® streptavidin, ALEXA FLUOR-647® streptavidin or unconjugated streptavidin and biotinylated TCRβ mAb were sequentially loaded onto the bilayer, while unlabeled poly-his-tagged ICAM1 produced in a baculovirus system was loaded to facilitate T cell adhesion.

Transduced T cells were sorted, rested 24-48 hrs, incubated with MGnBu (200 nM) in RPMI without serum or phenyl red for 15 mins. at 37° C., washed, resuspended in RPMI without serum or phenyl red and stimulated on a planar lipid bilayer. Imaging was performed using a Nikon Ti inverted microscope equipped with motorized TIRF arm and 100× 1.40 N.A. objective. Images were acquired at 100 ms intervals continuously for up to 15-20 mins with a Zyla sCMOS camera (Andor) equipped with bandpass emission filters for DAPI, FITC, TRITC, and Cy5 spectral profiles.

Images were analyzed using the NIS elements software version 5.21.00. Images were separated by layer and threshold for each layer was set. AF488 threshold was set between 200-1000, AF647 threshold was set between 150-40,000. Areas for individual channels and intersecting areas were measured using automated measurements.

Construction and characterization of chimeric antigen receptors: To generate the TIM3 CAR constructs (pHR-EF1-aCD19-TIM3ζ-T2A-TagBFP & pHR-EF1-aCD20-TIM3ζ-T2A-TagBFP), DNA encoding the human TIM-3 cytoplasmic domain was codon optimized, synthesized (Integrated DNA Technologies), and cloned into the pHR-EF1-αCD19-28ζ or pHR-EF1-αCD20-28ζ CAR lentiviral expression vectors, respectively, to replace the CD28 co-signaling domain using isothermal assembly.

To generate virus, HEK293T cells (ATCC) were transfected with pVSV-G (VSV glycoprotein expression plasmid), pMD2.G and the CAR-expression plasmid via calcium phosphate transfection (Clontech). At 16 hrs post-transfection cells were washed with PBS and incubated in complete DMEM media containing 6 mM sodium butyrate (Sigma Aldrich). Supernatants were collected at 24 and 48 hrs and were subsequently combined and filtered through a 45 μm vacuum filter. Viral particles were concentrated by ultracentrifugation for 1.5 hrs at 24,500 rpm, and viral pellets were re-suspended in 0.05 ml of supplemented RPMI medium and frozen at −80° C.

For CAR-T pS6 analysis, 100,000 Jurkat or primary T cells expressing the TIM-3 or CD28 CAR were incubated with an equal number of the indicated antigen positive or negative target cell line in a V-bottom plate for 30mins at 37° C. in 5% CO₂. Cells then underwent intracellular staining and flow cytometry as described as described (Avery et al., Proc Natl Acad Sci U S A 115, 2455-2460 (2018)).

CAR-T surface marker analysis: 100,000 Jurkat or Primary T cells expressing the TIM-3 or CD28 CAR were incubated with an equal number of the indicated antigen positive or negative target cell line in a V-bottom plate for 18 hrs at 37C in 5% CO2. Cells then underwent cell-surface antibody staining and flow cytometry (Avery et al., Proc Natl Acad Sci U S A 115, 2455-2460 (2018)).

### Example 10

DNA sequences of CARs

pHR-EF1-aCD20-TIM-3z-T2A-TagBFP (coding region) (SEQ ID NO: 19)
atggagacagacacactcctgctatgggtgctgctgctctgggttccaggttccacaggtGGTGACATTGTGCTGACCCAAT
CTCCAGCTATCCTGTCTGCATCTCCAGGGGAGAAGGTCACAATGACTTGCAGGGCCAGCTC
AAGTGTAAATTACATGGACTGGTACCAGAAGAAGCCAGGATCCTCCCCCAAACCCTGGATTT
ATGCCACATCCAACCTGGCTTCTGGAGTCCCTGCTCGCTTCAGTGGCAGTGGGTCTGGGAC
CTCTTACTCTCTCACAATCAGCAGAGTGGAGGCTGAAGATGCTGCCACTTATTACTGCCAGC
AGTGGAGTTTTAATCCACCCACGTTCGGAGGGGGGACCAAGCTGGAAATAAAAGGCAGTACT
AGCGGTGGTGGCTCCGGGGGCGGTTCCGGTGGGGGCGGCAGCAGCGAGGTGCAGCTGCA
GCAGTCTGGGGCTGAGCTGGTGAAGCCTGGGGCCTCAGTGAAGATGTCCTGCAAGGCTTCT
GGCTACACATTTACCAGTTACAATATGCACTGGGTAAAGCAGACACCTGGACAGGGCCTGGA
ATGGATTGGAGCTATTTATCCAGGAAATGGTGATACTTCCTACAATCAGAAGTTCAAAGGCAA
GGCCACATTGACTGCAGACAAATCCTCCAGCACAGCCTACATGCAGCTCAGCAGCCTGACAT
CTGAGGACTCTGCGGACTATTACTGTGCAAGATCTAATTATTACGGTAGTAGCTACTGGTTCT
TCGATGTCTGGGGCGCAGGGACCACGGTCACCGTCTCCTCAaccactactccggcaccgcgccccccaac
tcctgcaccgacgatagcttcacaaccgctttcattgcggcccgaagcatgtcggccagccgccggaggcgctgtgcatacaagagggct
ggattttgcatgtgatatatatatttgggcgccccttgctggcacttgcggcgttcttcttcttagcctcgttattacgctctactgtggcTTCAA
GTGGTATTCTCACTCCAAGGAAAAGATTCAAAACCTCTCATTGATTAGCCTTGCCAACCTTCC
CCCTAGCGGCCTCGCCAACGCGGTGGCCGAGGGGATCAGAAGTGAAGAAAACATCTACACC
ATCGAAGAAAATGTTTACGAAGTTGAAGAACCAAACGAATACTATTGTTACGTTAGCTCCCGA
CAACAGCCTTCACAGCCACTTGGCTGTCGCTTCGCCATGCCGcgggtgaagttcagcagaagcgccgac
gcccctgcctaccagcagggccagaatcagctgtacaacgagctgaacctgggcagaagggaagagtacgacgtcctggataagcgg
agaggccgggaccctgagatgggcggcaagcctcggcggaagaacccccaggaaggcctgtataacgaactgcAgaaagacaaga
tggccgaggcctacagcgagatcggcatgaagggcgagcggaggcggggcaagggccacgacggcctgtatcagggcctgtccacc
gccaccaaggatacctacgacgccctgcacatgcaggccctgcccccaaggctcgagggcggcggagagggcagaggaagtcttcta
acatgcggtgacGtggaggagaatcccggccctcgcATGAGCGAGCTGATTAAGGAGAACATGCACATGAAG
CTGTACATGGAGGGCACCGTGGACAACCATCACTTCAAGTGCACATCCGAGGGCGAAGGCA
AGCCCTACGAGGGCACCCAGACCATGAGAATCAAGGTGGTCGAGGGCGGCCCTCTCCCCTT
CGCCTTCGACATCCTGGCTACTAGCTTCCTCTACGGCAGCAAGACCTTCATCAACCACACCC
AGGGCATCCCCGACTTCTTCAAGCAGTCCTTCCCTGAGGGCTTCACATGGGAGAGAGTCAC
CACATACGAAGACGGGGGCGTGCTGACCGCTACCCAGGACACCAGCCTCCAGGACGGCTG
CCTCATCTACAACGTCAAGATCAGAGGGGTGAACTTCACATCCAACGGCCCTGTGATGCAGA
AGAAAACACTCGGCTGGGAGGCCTTCACCGAGACGCTGTACCCCGCTGACGGCGGCCTGG
AAGGCAGAAACGACATGGCCCTGAAGCTCGTGGGCGGGAGCCATCTGATCGCAAACATCAA
GACCACATATAGATCCAAGAAACCCGCTAAGAACCTCAAGATGCCTGGCGTCTACTATGTGG
ACTACAGACTGGAAAGAATCAAGGAGGCCAACAACGAAACATACGTCGAGCAGCACGAGGT
GGCAGTGGCCAGATACTGCGACCTCCCTAGCAAACTGGGGCACAAGCTTAATTAA
pHR-EF1-aCD19-TIM-3z-T2A-TagBFP (coding region) (SEQ ID NO: 20)
atggagacagacacactcctgctatgggtgctgctgctctgggttccaggttccacaggtGACATCCAGATGACACAGACTA
CATCCTCCCTGTCTGCCTCTCTGGGAGACAGAGTCACCATCAGTTGCAGGGCAAGTCAGGA
CATTAGTAAATATTTAAATTGGTATCAGCAGAAACCAGATGGAACTGTTAAACTCCTGATCTAC
CATACATCAAGATTACACTCAGGAGTCCCATCAAGGTTCAGTGGCAGTGGGTCTGGAACAGA
TTATTCTCTCACCATTAGCAACCTGGAGCAAGAAGATATTGCCACTTACTTTTGCCAACAGGG
TAATACGCTTCCGTACACGTTCGGAGGGGGGACCAAGCTGGAGATCACAGGTGGCGGTGGC
TCGGGCGGTGGTGGGTCGGGTGGCGGCGGATCTGAGGTGAAACTGCAGGAGTCAGGACCT
GGCCTGGTGGCGCCCTCACAGAGCCTGTCCGTCACATGCACTGTCTCAGGGGTCTCATTAC
CCGACTATGGTGTAAGCTGGATTCGCCAGCCTCCACGAAAGGGTCTGGAGTGGCTGGGAGT
AATATGGGGTAGTGAAACCACATACTATAATTCAGCTCTCAAATCCAGACTGACCATCATCAA
GGACAACTCCAAGAGCCAAGTTTTCTTAAAAATGAACAGTCTGCAAACTGATGACACAGCCAT
TTACTACTGTGCCAAACATTATTACTACGGTGGTAGCTATGCTATGGACTACTGGGGCCAAG
GAACCTCAGTCACCGTCTCCTCAaccactactccggcaccgcgccccccaactcctgcaccgacgatagcttcacaacc
gctttcattgcggcccgaagcatgtcggccagccgccggaggcgctgtgcatacaagagggctggattttgcatgtgatatatatatttgggc
gccccttgctggcacttgcggcgttcttcttcttagcctcgttattacgctctactgtggcTTCAAGTGGTATTCTCACTCCAAG
GAAAAGATTCAAAACCTCTCATTGATTAGCCTTGCCAACCTTCCCCCTAGCGGCCTCGCCAA
CGCGGTGGCCGAGGGGATCAGAAGTGAAGAAAACATCTACACCATCGAAGAAAATGTTTACG
AAGTTGAAGAACCAAACGAATACTATTGTTACGTTAGCTCCCGACAACAGCCTTCACAGCCAC
TTGGCTGTCGCTTCGCCATGCCGcgggtgaagttcagcagaagcgccgacgcccctgcctaccagcagggccagaatc
agctgtacaacgagctgaacctgggcagaagggaagagtacgacgtcctggataagcggagaggccgggaccctgagatgggcggc
aagcctcggcggaagaacccccaggaaggcctgtataacgaactgcAgaaagacaagatggccgaggcctacagcgagatcggcat
gaagggcgagcggaggcggggcaagggccacgacggcctgtatcagggcctgtccaccgccaccaaggatacctacgacgccctgc
acatgcaggccctgcccccaaggctcgagggcggcggagagggcagaggaagtcttctaacatgcggtgacGtggaggagaatcccg
gccctcgcATGAGCGAGCTGATTAAGGAGAACATGCACATGAAGCTGTACATGGAGGGCACCGT
GGACAACCATCACTTCAAGTGCACATCCGAGGGCGAAGGCAAGCCCTACGAGGGCACCCAG
ACCATGAGAATCAAGGTGGTCGAGGGCGGCCCTCTCCCCTTCGCCTTCGACATCCTGGCTA
CTAGCTTCCTCTACGGCAGCAAGACCTTCATCAACCACACCCAGGGCATCCCCGACTTCTTC
AAGCAGTCCTTCCCTGAGGGCTTCACATGGGAGAGAGTCACCACATACGAAGACGGGGGCG
TGCTGACCGCTACCCAGGACACCAGCCTCCAGGACGGCTGCCTCATCTACAACGTCAAGAT
CAGAGGGGTGAACTTCACATCCAACGGCCCTGTGATGCAGAAGAAAACACTCGGCTGGGAG
GCCTTCACCGAGACGCTGTACCCCGCTGACGGCGGCCTGGAAGGCAGAAACGACATGGCC
CTGAAGCTCGTGGGCGGGAGCCATCTGATCGCAAACATCAAGACCACATATAGATCCAAGAA
ACCCGCTAAGAACCTCAAGATGCCTGGCGTCTACTATGTGGACTACAGACTGGAAAGAATCA
AGGAGGCCAACAACGAAACATACGTCGAGCAGCACGAGGTGGCAGTGGCCAGATACTGCGA
CCTCCCTAGCAAACTGGGGCACAAGCTTAATTAA
Translated AA sequences of CARs
pHR-EF1-aCD20-TIM-3z-T2A-TagBFP (coding region) (SEQ ID NO: 21)
METDTLLLWVLLLWVPGSTGGDIVLTQSPAILSASPGEKVTMTCRASSSVNYMDWYQKKP
GSSPKPWIYATSNLASGVPARFSGSGSGTSYSLTISRVEAEDAATYYCQQWSFNPPTFGGGT
KLEIKGSTSGGGSGGGSGGGGSSEVQLQQSGAELVKPGASVKMSCKASGYTFTSYNMHW
VKQTPGQGLEWIGAIYPGNGDTSYNQKFKGKATLTADKSSSTAYMQLSSLTSEDSADYYC
ARSNYYGSSYWFFDVWGAGTTVTVSSTTTPAPRPPTPAPTIASQPLSLRPEACRPAAGGAVHT
RGLDFACDIYIWAPLAGTCGVLLLSLVITLYCGFKWYSHSKEKIQNLSLISLANLPPSGLANAV
AEGIRSEENIYTIEENVYEVEEPNEYYCYVSSRQQPSQPLGCRFAMPRVKFSRSADAPAYQQ
GQNQLYNELNLGRREEYDVLDKRRGRDPEMGGKPRRKNPQEGLYNELQKDKMAEAYSEIGMK
GERRRGKGHDGLYQGLSTATKDTYDALHMQALPPRLEGGGEGRGSLLTCGDVEENPGPR
MSELIKENMHMKLYMEGTVDNHHFKCTSEGEGKPYEGTQTMRIKVVEGGPLPFAFD
ILATSFLYGSKTFINHTQGIPDFFKQSFPEGFTWERVTTYEDGGVLTATQDTSLQDGC
LIYNVKIRGVNFTSNGPVMQKKTLGWEAFTETLYPADGGLEGRNDMALKLVGGSHL
IANIKTTYRSKKPAKNLKMPGVYYVDYRLERIKEANNETYVEQHEVAVARYCDLPSK
LGHKLN*

### Annotations

Leader sequence
Leu16 scFv (CD20 binding region)
CD8alpha spacer and transmembrane
TIM-3 cytoplasmic domain
CD3zeta-cytoplasmic domain
T2A-TagBFP marker gene
pHR-EF1-aCD19-TIM-3z-T2A-TagBFP (coding region) (SEQ ID NO: 22)
METDTLLLWVLLLWVPGSTGDIQMTQTTSSLSASLGDRVTISCRASQDISKYLNWYQQKP
DGTVKLLIYHTSRLHSGVPSRESGSGSGTDYSLTISNLEQEDIATYFCQQGNTLPYTFGGGT
KLEITGGGGSGGGGSGGGGSEVKLQESGPGLVAPSQSLSVTCTVSGVSLPDYGVSWIRQPP
RKGLEWLGVIWGSETTYYNSALKSRLTIIKDNSKSQVFLKMNSLQTDDTAIYYCAKHYYY
GGSYAMDYWGQGTSVTVSSTTTPAPRPPTPAPTIASQPLSLRPEACRPAAGGAVHTRGLDFAC
DIYIWAPLAGTCGVLLLSLVITLYCGFKWYSHSKEKIQNLSLISLANLPPSGLANAVAEGIRSEE
NIYTIEENVYEVEEPNEYYCYVSSRQQPSQPLGCRFAMPRVKFSRSADAPAYQQGQNQLYNE
LNLGRREEYDVLDKRRGRDPEMGGKPRRKNPQEGLYNELQKDKMAEAYSEIGMKGERRRGKG
HDGLYQGLSTATKDTYDALHMQALPPRLEGGGEGRGSLLTCGDVEENPGPRMSELIKEN
MHMKLYMEGTVDNHHFKCTSEGEGKPYEGTQTMRIKVVEGGPLPFAFDILATSFLY
GSKTFINHTQGIPDFFKQSFPEGFTWERVTTYEDGGVLTATQDTSLQDGCLIYNVKIR
GVNFTSNGPVMQKKTLGWEAFTETLYPADGGLEGRNDMALKLVGGSHLIANIKTTY
RSKKPAKNLKMPGVYYVDYRLERIKEANNETYVEQHEVAVARYCDLPSKLGHKLN*
Annotations:
Leader sequence
FMC63 scFv (CD19 binding region)
CD8alpha spacer and transmembrane
TIM-3 cytoplasmic domain
CD3zeta-cytoplasmic domain
T2A-TagBFP marker gene

In view of the many possible embodiments to which the principles of our invention may be applied, it should be recognized that illustrated embodiments are only examples of the invention and should not be considered a limitation on the scope of the invention. Rather, the scope of the invention is defined by the following claims. We therefore claim as our invention all that comes within the scope and spirit of these claims.

1. A chimeric antigen receptor comprising:
(a) an extracellular scFv comprising a light chain variable domain (V_{L}) and a heavy chain variable domain (V_{H}), wherein the scFv specifically binds to an antigen of interest;
(b) a CD8 hinge domain and transmembrane domain; and
(c) a cytoplasmic domain comprising (i) a TIM-3 cytoplasmic domain and (ii) an intracellular signaling domain,
wherein (a)-(c) are in N-terminal to C-terminal order.

2. The chimeric antigen receptor of claim 1, wherein the intracellular signaling domain is a 4-1BB, CD3 zeta, FcR gamma, FcR beta, CD3 gamma, CD3 delta, CD3 epsilon, CD22, CD22, CD79a, or CD79b intracellular signaling domain.

3. The chimeric antigen receptor of claim 1, wherein the intracellular signaling domain is a CD3 zeta intracellular signaling domain.

4. The chimeric antigen receptor of claim 1, wherein the TIM-3 cytoplasmic domain comprises or consist of one of SEQ ID NOs: 10, 11, 12, 13, 34, 36, 37, 38, 39 or 40.

5. The chimeric antigen receptor of claim 4, wherein the antigen of interest is a tumor antigen.

6. The chimeric antigen receptor of claim 5, wherein the tumor antigen is CD22, CD123, CEA, EGFRVIII, ErbB2, HER2, IL-13ralpha2, MUC1, CD19 or CD20.

7. The chimeric antigen receptor of claim 1, further comprising an additional intracellular co-stimulatory signaling domain a) C-terminal to the TIM-3 cytoplasmic domain or b) between the TIM-3 cytoplasmic domain and the intracellular signaling domain.

8. The chimeric antigen receptor of claim 7, wherein the intracellular co-stimulatory signaling domain is a CD27, CD28, 4-1BB (CD137), OX₄₀ (CD134), CD30, CD40, PD-1, ICOS, lymphocyte function-associated antigen 1 (LFA-1), CD2, CD7, or B7-H3 intracellular co-stimulatory signaling domain.

9. The chimeric antigen receptor of claim 6, wherein the intracellular co-stimulatory signaling domain is a 4-1BB intracellular signaling domain.

10. The chimeric antigen receptor of claim 1, comprising the amino acid sequence of SEQ ID NOs: 21 or 22.

11. An isolated nucleic acid molecule encoding the chimeric antigen receptor of claim 1.

12. The isolated nucleic acid of claim 11, operably linked to a promoter.

13. An expression vector comprising the nucleic acid molecule of claim 11.

14. The expression vector of claim 13, wherein the vector is a viral vector.

15. The expression vector of claim 14, wherein the viral vector is a lentiviral vector or a gamma retroviral vector.

16. A CD3⁺ T cell or natural killer cell transduced with the expression vector of claim 13.

17. The CD3⁺ T cell of claim 16, wherein the CD3⁺ T cells is a CD3⁺CD4⁺ T cell or a CD3⁺CD8⁺ T cell.

18. The CD3⁺ T cell or natural killer cell of claim 16, wherein the T cell or natural killer cell is a human T cell.

19. A pharmaceutical composition comprising an effective amount of the expression vector of claim 13, or a therapeutically effective amount of a CD3+ T cell and/or a natural killer cell comprising the expression vector, and a pharmaceutically acceptable carrier.

20. A method for treating a subject with a malignancy that expresses an antigen of interest, comprising administering to the subject a therapeutically effective amount of the pharmaceutical composition of claim 19, thereby treating the malignancy.

21. The method of claim 20, wherein the pharmaceutical composition comprises the T cells.

22. The method of claim 21, wherein the T cells are autologous to the subject.

23. The method of claim 21, wherein the pharmaceutical composition comprises CD3⁺CD4⁺ T cells and/or CD3⁺CD8⁺ T cells.

24. The method of claim 20, wherein the subject is human.

25. The method of claim 20, wherein the antigen of interest is CD20 or CD19, and the malignancy is a leukemia.

26-27. (canceled)
